(19) United States
(12) Patent Application Publication
(10) Pub. No.: US 2022/0272806 A1
(43) Pub. Date: Aug. 25, 2022

(54) LED LIGHTING CHANNELS HAVING SPECTRAL POWER DISTRIBUTION CHARACTERISTICS AND RELATED MULTI-CHANNEL TUNABLE WHITE LIGHTING SYSTEMS

(71) Applicant: Ecosense Lighting Inc., Los Angeles, CA (US)

(72) Inventors: Raghuram L. V. Petluri, Los Angeles, CA (US); Paul Kenneth PICKARD, Los Angeles, CA (US)

(21) Appl. No.: 17/667,807

(22) Filed: Feb. 9, 2022

Related U.S. Application Data
(63) Continuation of application No. PCT/US20/45626, filed on Aug. 10, 2020.
(60) Provisional application No. 62/885,162, filed on Aug. 9, 2019.

(51) Int. Cl. H05B 45/20

(52) U.S. Cl. CPC H05B 45/20

(57) ABSTRACT

A tunable lighting system comprising a plurality of channels comprising at least, a first channel for emitting blue light and having a wavelength peak between 420 nm and 480 nm, a second channel for emitting cyan light having a wavelength peak between 450 nm and 530 nm, a third channel for emitting cyan-green light having a wavelength peak between 510 nm and 590 nm, a fourth channel for emitting red light having a wavelength peak between 510 nm and 780 nm, and a multichannel driver for driving a selection of said plurality of channels, said multichannel driver is configured to drive each channel independently such that said light system emits an emitted light with a CRI of at least 85 over a CCT range greater than 3000 K.

### REFERENCE TO RELATED APPLICATIONS

This application is based on U.S. Provisional Application No. 62/885,162, filed Aug. 9, 2019, hereby incorporated by reference in its entirely, including its Appendix

### FIELD OF DISCLOSURE

This disclosure is in the field of solid-state lighting. In particular, the disclosure relates to devices for use in, and methods of, providing tunable white light with high color rendering performance.

### BACKGROUND

The quality of light emitted from a light emitting diode (LED) may be described in various ways. For example, the 1931 CIE (Commission Internationale de l'Éclairage) Chromaticity Diagram maps out the human color perception in terms of two CIE parameters x and y. FIG. 3 illustrates a 1931 International Commission on Illumination (CIE) chromaticity diagram. The 1931 CIE Chromaticity diagram is a two-dimensional chromaticity space in which every visible color is represented by a point having x- and y-coordinates, also referred to herein as (ccx, ccy) coordinates. Unless otherwise specified herein, all chromaticity coordinates discloses herein pertain to the 1931 CIE Chromaticity Diagram.

Fully saturated (monochromatic) colors appear on the outer edge of the diagram, while less saturated colors (which represent a combination of wavelengths) appear on the interior of the diagram. The term “saturated”, as used herein, means having a purity of at least 85%, the term “purity” having a well-known meaning to persons skilled in the art, and procedures for calculating purity being well-known to those of skill in the art. The Planckian locus, or black body locus (BBL), represented by line 150 on the diagram, follows the color an incandescent black body would take in the chromaticity space as the temperature of the black body changes from about 1,000 K to 10,000 K. The black body locus goes from deep red at low temperatures (about 1,000 K) through orange, yellowish white, white, and finally bluish white at very high temperatures. The temperature of a black body radiator corresponding to a particular color in a chromaticity space is referred to as the “correlated color temperature.” In general, light corresponding to a correlated color temperature (CCT) of about 2700 K to about 6500 K. is considered to be “white” light. In particular, as used herein, “white light” generally refers to light having a chromaticity point that is within a 10-step MacAdam ellipse of a point on the black body locus having a CCI between 2700 K and 6500 K. However, it will be understood that tighter or looser definitions of white light can be used if desired. For example, white light can refer to light having a chromaticity point that is within a seven step MacAdam ellipse of a point on the black body locus having a CCT between 2700 K and 6500 K.

The distance from the black body locus can be measured in the CIE 1960 chromaticity diagram, and is indicated by the symbol Δuv, or DUV or duv as referred to elsewhere herein. If the chromaticity point is above the Planckian locus the DUV is denoted by a positive number, and if the chromaticity point is below the locus, DUV is indicated with a negative number. If the DUV is sufficiently positive, the light source may appear greenish or yellowish at the same CCT. If the MTV is sufficiently negative, the light source can appear to be purple or pinkish at the same CCT. Observers may prefer light above or below the Planckian locus for particular CCT values. DUV calculation methods are well known by those of ordinary skill in the art and are more fully described in ANSI 078.377, American National Standard for Electric Lamps—Specifications for the Chromaticity of Solid State Lighting (SSL) Products, which is incorporated by reference herein in its entirety for all purposes. A point representing the CIF Standard Illuminant D65 is also shown on the diagram. The D65 illuminant is intended to represent average daylight and has a CCT of approximately 6500 K and the spectral power distribution is described more fully in Joint ISO/CIE Standard, ISO 10526:1999/CIE S5/E-1998, CIE Standard Illuminants for Colorimetry, which is incorporated by reference herein in its entirety for all purposes.

The ability of a light source to accurately reproduce color in illuminated objects can be characterized using the color rendering index (“CRI”), also referred to as the CIE Ra value. The Ra value of a light source is a modified average of the relative measurements of how the color rendition of an illumination system compares to that of a reference black-body radiator or daylight spectrum when illuminating eight reference colors R1-R8. Thus, the Ra value is a relative measure of the shift in surface color of an object when lit by a particular lamp. The Ra value equals 100 if the color coordinates of a set of test colors being illuminated by the illumination system are the same as the coordinates of the same test colors being irradiated by a reference light source of equivalent CU. For CCTs less than 5000 K, the reference illuminants used in the CRI calculation procedure are the SPDs of blackbody radiators; for CCTs above 5000 K, imaginary SPDs calculated from a mathematical model of daylight are used. These reference sources were selected to approximate incandescent lamps and daylight, respectively. Daylight generally has an Ra value of nearly 100, incandescent bulbs have an Ra value of about 95, fluorescent lighting typically has an Ra value of about 70 to 85, while monochromatic light sources have an Ra value of essentially zero. Light sources for general illumination applications with an Ra value of less than 50 are generally considered very poor and are typically only used in applications where economic issues preclude other alternatives. The calculation of CIE Ra values is described more fully in Commission Internationale de l'Éclairage. 1995. Technical Report: Method of Measuring and Specifying

Colour Rendering Properties of Light Sources, CIE No. 13.3-1995. Vienna, Austria: Commission Internationale de l'Éclairage, which is incorporated by reference herein in its entirety for all purposes. In addition to the Ra value, a light source can also be evaluated based on a measure of its ability to render seven additional colors R9-R15, which include realistic colors like red, yellow, green, blue, caucasian skin color (R13), tree leaf green, and Asian skin color (R15), respectively. The ability to render the saturated red reference color R9 can be expressed with the R9 color rendering value (“R9 value”), Light sources can further be evaluated by calculating the gamut area index (“GAI”). Connecting the rendered color points from the determination of the CIE Ra value in two dimensional space will form a gamut area.

Gamut area index is calculated by dividing the gamut area formed by the light source with the gamut area formed by a reference source using the same set of colors that are used for CRI. GAI uses an Equal Energy Spectrum as the reference source rather than a black body radiator. A gamut area index related to a black body radiator (“GAIBB”) can be calculated by using the gamut area formed by the blackbody radiator at the equivalent CCI to the light source.

The ability of a light source to accurately reproduce color in illuminated objects can be characterized using the metrics described in IES Method for Evaluating Light Source Color Rendition, Illuminating Engineering Society, Product ID: TM-30-15 (referred to herein as the “TM-30-15 standard”), which is incorporated by reference herein in its entirety for all purposes. The TM-30-15 standard describes metrics including the Fidelity Index (Rf) and the Gamut Index (Rg) that can be calculated based on the color rendition of a light source for 99 color evaluation samples (“CES”). The 99 CES provide uniform color space coverage, are intended to be spectral sensitivity neutral, and provide color samples that correspond to a variety of real objects. Rf values range from 0 to 1 and indicate the fidelity with which a light source renders colors as compared with a reference illuminant. Rg values provide a measure of the color gamut that the light source provides relative to a reference illuminant. The range of Rg depends upon the Rf value of the light source being tested. The reference illuminant is selected depending on the CCT. For CCT values less than or equal to 4500 K, Planckian radiation is used. For CCT values greater than or equal to 5500 K, CIE Daylight illuminant is used. Between 4500 K and 5500 K a proportional mix of Planckian radiation and the CIE. Daylight illuminant is used, according to the following equations:

$${S}_{r , M} ( λ , {T}_{t} ) = \frac{5500 - {T}_{t}}{1000} {S}_{r , P} ( λ , {T}_{t} ) + ( 1 - \frac{5500 - {T}_{t}}{1000} ) {S}_{r , D} ( {λ}_{,} {T}_{t} ) ,$$

where Tₜ is the CCT value, S_{r,M}(λ, Tₜ) is the proportional mix reference illuminant, S_{r,P}(λ, Tₜ) is Planckian radiation, and S_{r,D}(λ, Tₜ) is the CIE Daylight illuminant.

The ability of a light source to provide illumination that allows for the clinical observation of cyanosis is based upon the light source's spectral power density in the red portion of the visible spectrum, particularly around 660 nm. The cyanosis observation index (“COI”) is defined by AS/NZS 1680.2.5 Interior Lighting Part 2.5: Hospital and Medical Tasks, Standards Australia, 1997 which is incorporated by reference herein in its entirety, including all appendices, for all purposes. COI is applicable for CCTs from about 3300 K to about 5500 K, and is preferably of a value less than about 3.3. If a light source's output around 660 nm is too low, a patient's skin color may appear darker and may be falsely diagnosed as cyanosed. if a light source's output at 660 nm is too high, it may mask any cyanosis, and it may not be diagnosed when it is present. COI is a dimensionless number and is calculated from the spectral power distribution of the light source. The COI value is calculated by calculating the color difference between blood viewed under the test light source and viewed under the reference lamp (a 4000 K Planckian source) for 50% and 100% oxygen saturation and averaging the results. The lower the value of COI, the smaller the shift in color appearance results under illumination by the source under consideration.

The spectral profiles of light emitted by white artificial lighting can impact circadian physiology, alertness, and cognitive performance levels. Bright artificial light can be used in a number of therapeutic applications, such as in the treatment of seasonal affective disorder (SAD), certain sleep problems, depression, jet lag, sleep disturbances in those with Parkinson's disease, the health consequences associated with shift work, and the resetting of the human circadian clock. Artificial lighting may change natural processes, interfere with melatonin production, or disrupt the circadian rhythm. Blue light may have a greater tendency than other colored light to affect living organisms through the disruption of their biological processes which can rely upon natural cycles of daylight and darkness. Exposure to blue light late in the evening and at night may be detrimental to one's health. Some blue or royal blue light within lower wavelengths can have hazardous effects to human eyes and skin, such as causing damage to the retina.

Circadian stimulation can be quantified in different ways. For example, Circadian illuminance (CLA) is a measure of circadian effective light, spectral irradiance distribution of the light incident at the cornea weighted to reflect the spectral sensitivity of the human circadian system as measured by acute melatonin suppression after a one-hour exposure, and CS, which is the effectiveness of the spectrally weighted irradiance at the cornea from threshold (CS=0.1) to saturation (CS=0.7). The values of CLA are scaled such that an incandescent source at 2856 K (known as CIE Illuminant A) which produces 1000 lux (visual lux) will produce 1 units of circadian lux (CLA). CS values are transformed CLA values and correspond to relative melotonian suppression after one hour of light exposure for a 2.3 mm diameter pupil during the mid-point of melotonian production. CS is calculated as follows:

$$CS = ( 1 - \frac{1}{1 + {( \frac{CLA}{355.7} )}^{⋀ 1.126}} ) .$$

The calculation of CLA is more fully described in Rea et al., “Modelling the spectral sensitivity of the human circadian system,” Lighting Research and Technology, 2011; 0: 1-12, and Figueiro et al., “Designing with Circadian Stimulus”, October 2016, LD+A Magazine, Illuminating Engineering Society of North. America, which are incorporated by reference herein in its entirety for all purposes. Figueiro et al. describe that exposure to a CS of 0.3 or greater at the eve, for at least one hour in the early part of the day, is effective for stimulating the circadian system and is associated with better sleep and improved behavior and mood.

Equivalent Melanopic Lux (EML) provides a measure of photoreceptive input to circadian and neurophysiological light responses in humans, as described in Lucas et al., “Measuring and using light in the melanopsin age.” Trends in Neurosciences, January 2014, Vol. 37, No. 1, pages 1-9, which is incorporated by reference herein in its entirety, including all appendices, for all purposes. Melanopic lux is weighted to a photopigment with λmax 480 nm with pre-receptoral filtering based on a 32 year old standard observer, as described more fully in the Appendix A, Supplementary Data to Lucas et al. (2014), User Guide: Irradiance Toolbox (Oxford 18 Oct. 2013), University of Manchester, Lucas Group, which is incorporated by reference herein in its entirety for all purposes. EML values are shown in the tables and Figures herein as the ratio of melanopic lux to luminous flux, with luminous flux considered to be 1 lumens. It can be desirable for biological effects on users to provide illumination having higher EML in the morning, but lower EML in the late afternoon and evening.

Another circadian quantification is described in Ji Hye Oh, Su Ji Yang and Young Rag Do, “Healthy, natural, efficient and tunable lighting: four-package white LEDs for optimizing the circadian effect, color quality and vision performance,” Light: Science & Applications (2014) 3: e141-e149, which is incorporated herein in its entirety, including supplementary information, for all purposes. Luminous efficacy of radiation (“LER”) can be calculated from the ratio of the luminous flux to the radiant flux (S(λ)), i.e. the spectral power distribution of the light source being evaluated, with the following equation:

$$L E R ( \frac{lm}{W} ) = 683 ( \frac{lm}{W} ) \frac{\int V ( λ ) S ( λ ) d λ}{\int S ( λ ) d λ} .$$

Circadian efficacy of radiation (“CER”) can be calculated from the ratio of circadian luminous flux to the radiant flux, with the following equation:

$$CER ( \frac{bim}{W} ) = 683 ( \frac{lm}{W} ) \frac{\int C ( λ ) S ( λ ) d λ}{\int S ( λ ) d λ} .$$

Circadian action factor (“CAE”) can be defined by the ratio of CER. to LER, with the following equation:

$$( \frac{blm}{lm} ) = \frac{CER ( \frac{blm}{W} )}{LER ( \frac{lm}{W} )} .$$

The term “blm” refers to biolumens, units for measuring circadian flux, also known as circadian lumens. The term “lm” refers to visual lumens. V(λ) is the photopic spectral luminous efficiency function and C(λ) is the circadian spectral sensitivity function.

The calculations herein use the circadian spectral sensitivity function, C λ, from Gall et al., Proceedings of the CIE Symposium 24 on Light and Health: Non-Visual Effects, 30 Sep.-2 Oct. 2024; Vienna, Austria 24. CIF Wien, 24, pp 129-132, which is incorporated herein in its entirety for all purposes.

By integrating the amount of light (milliwatts) within the circadian spectral sensitivity function and dividing such value by the number of photopic lumens, a relative measure of melatonin suppression effects of a particular light source can be obtained. A scaled. relative measure denoted as melatonin suppressing milliwatts per hundred lumens may be obtained by dividing the photopic lumens by the term “melatonin suppressing milliwatts per hundred lumens” consistent with the foregoing calculation method is used throughout this application and the accompanying figures and tables.

Blue Light Hazard (BLH) provides a measure of potential for a photochemical induced retinal injury that results from radiation exposure. Blue Light Hazard is described in IEC/EN 62471, Photobiological Safety of Lamps and Lamp Systems and Technical Report IEC/TR 62778: Application of IEC 62471 for the assessment of blue light hazard to light sources and luminaires, which are incorporated by reference herein in their entirety for all purposes. A BLH factor can be expressed in (weighted power/lux) in units of μW/cm2/lux.

LED lamps have been provided that can emit white light with different CCI values within a range. Such lamps often utilize two or more LEDs, with or without luminescent materials, with respective drive currents that are increased or decreased to increase or decrease the amount of light emitted by each LED, By controllably altering the power to the various LEDs in the lamp, the overall light emitted can be tuned to different CCT values. The range of CCT values that can be provided with adequate color rendering values and efficiency is limited by the selection of LEDs. Thus, there is a need to provide LED lamps that can provide white light across a range of CCT values while simultaneously achieving high efficiencies, high luminous flux, good color rendering, and acceptable color stability. There is also a need to provide lighting apparatuses that can provide desirable lighting performance while allowing for the control of circadian energy performance. The present invention fulfills these needs among others.

### DISCLOSURE

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

In one embodiment, the invention relates to a tunable lighting system having at least four unsaturated spectrum-configured channels, which are selectively powered such that the light system emits light having a high CRI value (e.g., greater than 85) over a wide CCT range (e.g., greater than 3000 K). In one embodiment, the tunable lighting system comprises: (a) a plurality of channels comprising at least, (i) a first channel for emitting blue light and having a wavelength peak between 420 nm and 480 nm; (ii) a second channel for emitting cyan light having a wavelength peak between 450 nm and 530 nm; (iii) a third channel for emitting cyan-green light having a wavelength peak between 510 nm and 590 nm; and (iv) a fourth channel for emitting red light having a wavelength peak between 510 nm and 780 nm; and (b) a multichannel driver for driving a selection of said plurality of channels, said multichannel driver is configured to drive each channel independently such that said light system emits an emitted light with a CRI of at least 85 over a CCT range greater than 3000 K.

In one embodiment, the invention relates to the spectrum configuration of each channel.

In one embodiment, the invention relates to modes of operating the channels to emit white light. In certain embodiments, substantially the same white light points, with similar CCT values, can be generated in different operating modes that each utilize different combinations of the blue, red, short-blue-pumped cyan, and long-blue-pumped cyan channels of the disclosure. In some embodiments, a first operating mode can use the blue, red, and short-blue-pumped cyan channels (also referred to herein as a “High-CM mode”) and a second operating mode can use the blue, red, and long-blue-pumped cyan channels of a device (also referred to herein as a “High-EMI, mode”).

### DRAWINGS

The summary, as well as the following detailed description, is further understood when read in conjunction with the appended drawings. For the purpose of illustrating the disclosure, there are shown in the drawings exemplary embodiments of the disclosure; however, the disclosure is not limited to the specific methods, compositions, and devices disclosed. In addition, the drawings are not necessarily drawn to scale. In the drawings:

FIG. 1 illustrates aspects of light emitting devices according to the present disclosure;

FIG. 2 illustrates aspects of light emitting devices according to the present disclosure;

FIG. 3 depicts a graph of a 1931 CIE Chromaticity Diagram illustrating the location of the Planckian locus;

FIGS. 4A-4B illustrate some aspects of light emitting devices according to the present disclosure, including some suitable color ranges for light generated by components of the devices;

FIG. 5 illustrates some aspects of light emitting devices according to the present disclosure, including some suitable color ranges for light generated by components of the devices;

FIG. 6 illustrates some aspects of light emitting devices according to the present disclosure, including some suitable color ranges for light generated by components of the devices;

FIG. 7 illustrates some aspects of light emitting devices according to the present disclosure, including some suitable color ranges for light generated by components of the devices;

FIG. 8 illustrates some aspects of light emitting devices according to the present disclosure, including some suitable color ranges for light generated by components of the devices;

FIG. 9 illustrates some aspects of light emitting devices according to the present disclosure, including some suitable color ranges for light generated by components of the devices;

FIG. 10 illustrates some aspects of light emitting devices according to the present disclosure, including some suitable color ranges for light generated by components of the devices;

FIG. 11 illustrates aspects of light emitting devices according to the present disclosure;

FIG. 12 illustrates some aspects of light emitting devices according to the present disclosure, including some suitable color points for light generated by components of the devices;

FIG. 13 illustrates some aspects of light emitting devices according to the present disclosure, including sonic suitable color ranges for light generated by components of the devices;

FIG. 14A and FIG. 14B illustrate some aspects of light emitting devices according to the present disclosure, including sonic suitable color ranges for light generated by components of the devices;

FIG. 15 illustrates some aspects of light emitting devices according to the present disclosure in comparison with some prior art and some theoretical light sources, including some light characteristics of white light generated by light emitting devices in various operational modes;

FIG. 16 illustrates some aspects of light emitting devices according to the present disclosure, including aspects of spectral power distributions for light generated by components of the devices;

FIG. 17 illustrates some aspects of light emitting devices according to the present disclosure, including aspects of spectral power distributions for light generated by components of the devices;

FIG. 18 illustrates some aspects of light emitting devices according to the present disclosure, including aspects of spectral power distributions for light generated by components of the devices;

FIG. 19 illustrates some aspects of light emitting devices according to the present disclosure, including aspects of spectral power distributions for light generated by components of the devices;

FIG. 20 illustrates some aspects of light emitting devices according to the present disclosure, including aspects of spectral power distributions for light generated by components of the devices;

FIG. 21 illustrates some aspects of light emitting devices according to the present disclosure, including aspects of spectral power distributions for light generated by components of the devices;

### FURTHER DISCLOSURE

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular exemplars by way of example only and is not intended to be limiting of the claimed disclosure.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another exemplar includes from the one particular value and/or to the other particular value. Similarly, when values are expressed as approximations, by use of the antecedent “about,” it will be understood that the particular value forms another exemplar. All ranges are inclusive and combinable.

It is to be appreciated that certain features of the disclosure which are, for clarity, described herein in the context of separate exemplar, may also be provided in combination in a single exemplary implementation. Conversely, various features of the disclosure that are, for brevity, described in the context of a single exemplary implementation, may also he provided separately or in any subcombination. Further, reference to values stated in ranges include each and every value within that range.

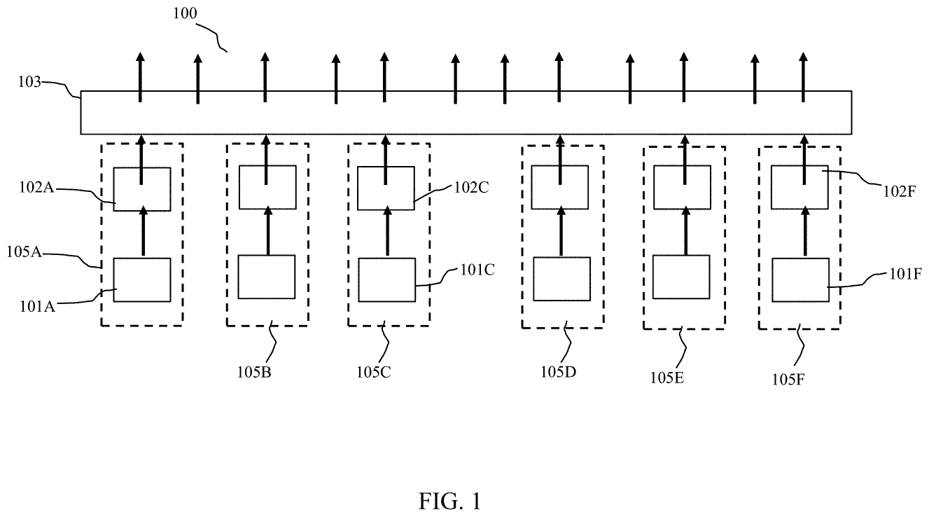

In one aspect, the present disclosure provides semiconductor light emitting devices 1 that can have a plurality of light emitting diode (LED) strings. Each LED string can have one, or more than one, LED. As depicted schematically in FIG. 1, the device 100 may comprise a plurality of lighting channels 105A-F formed from LED strings 101A-F and optionally with associated luminophoric mediums 102A-F to produce a particular light output from each of the lighting channels 105A-F. Each lighting channel can have an LED string (101A-F) that emits light (schematically shown with arrows). In some instances, the LED strings can have recipient luminophoric mediums (102A-F) associated therewith. The light emitted from the LED strings, combined with light emitted from the recipient luminophoric mediums, can be passed through one or more optical elements 103. Optical elements 103 may be one or more diffusers, lenses, light guides, reflective elements, or combinations thereof. In some embodiments, one or more of the LED strings 101A-F may be provided without an associated luminophoric medium.

A recipient luminophoric medium 102A-F includes one or more luminescent materials and is positioned to receive light that is emitted by an LED or other semiconductor light emitting device. In some embodiments, recipient luminophoric mediums include layers having luminescent materials that are coated or sprayed directly onto a semiconductor light emitting device or on surfaces of the packaging thereof, and clear encapsulants that include luminescent materials that are arranged to partially or fully cover a semiconductor light emitting device. A recipient luminophoric medium may include one medium layer or the like in which one or more luminescent materials are mixed, multiple stacked layers or mediums, each of which may include one or more of the same or different luminescent materials, and/or multiple spaced apart layers or mediums, each of which may include the same or different luminescent materials. Suitable encapsulants are known by those skilled in the art and have suitable optical, mechanical, chemical, and thermal characteristics. In some embodiments, encapsulants can include dimethyl silicone, phenyl silicone, epoxies, acrylics, and polycarbonates. In some embodiments, a recipient luminophoric medium can be spatially separated (i.e., remotely located) from an or surfaces of the packaging thereof. In some embodiments, such spatial segregation may involve separation of a distance of at least about 1 mm, at least about 2 mm, at least about 5 mm, or at least about 10 mm. In certain embodiments, conductive thermal communication between a spatially segregated luminophoric medium and one or more electrically activated emitters is not substantial. Luminescent materials can include phosphors, scintillators, day glow tapes, nanophosphors, inks that glow in visible spectrum upon illumination with light, semiconductor quantum dots, or combinations thereof. In some embodiments, the luminescent materials may comprise phosphors comprising one or more of the following materials: BaMg2Al16O27:Eu2+, BaMg2Al16O27:Eu2+,Mn2+, CaSiO3:Pb,Mn, CaWO4:Pb, MgWO4, Sr5Cl(PO4)3:Eu2+, Sr2P2O7:Sn2+, Sr6P5BO20:Eu, CaSF(PO4)3:Sb, (Ba,Ti)2P2O7:Ti, Sr5F(PO4)3:SbAln, (La,Ce,Tb)PO4:Ce,Tb, (Ca,Zn,Mg)3(PO4)2:Sn, (Sr,Mg)3(PO4)2:Sn, Y2O3:Eu3+, Mg4(F)GeO6:Mn, LaMgAl11O19:Ce, LaPO4:Ce, SrAl12O19:Ce, BaSi2O5:Pb, SrB4O7:Eu, Sr2MgSi2O7:Pb, Gd2O2S:Tb, Gd2O2S:Eu, Gd2O2S:Pr, Gd2O2S:Pr,Ce,F,Y2O2S:Tb, Y2O2S:Eu, Y2O2S:Pr, Zn(0.5)Cd(0.4)S:Ag, Zn(0.4)Cd(0.6)S:Ag, Y2SiO5:Ce, YAlO3:Ce, 3(Al,Ga)5O12:Ce, CdS:In, ZnO:Ga, ZnO:Zn, (Zn,Cd)S:Cu,Al, ZnCdS:Ag,Cu, ZnS:Ag, ZnS:Cu, NaLTl, CsI, 6LiF/ZnS:Ag, 6LiF/ZnS:Cu,Al,Au,ZnS:Cu,Al, ZnS:Cu,Au,Al, CaAlSiN3:Eu, (Sr,Ca)AlSiN3:Eu, (Ba,Ca,Sr,Mg)2SiO4:Eu, Lu3Al5O12:Ce, Eu3+(Gd0.9Y0.1)3Al5O12:Bi3+,Tb3+, Y3Al5O12:Ce, (La,Y)3SiGN11:Ce, Ca2AlSi3O2N5:Ce3+, Ca2AlSi3O2N5:Eu2+, BaMgAl10O17:Eu, Sr5(PO4)3Cl: Eu, (Ba,Ca,Sr,Mg)2SiO4:Eu, Si6-zAlzN8-zOz:Eu (wherein 0<z≤4.2); M3Si6O12N2:Eu (wherein M=alkaline earth metal element), (Mg,Ca,Sr,Ba)Si2O2N2:Eu, Sr4Al14O25:Eu, (Ba,Sr,Ca),Al2O4:Eu, (Sr,Ba),Al2Si2O8:Eu, (Ba,Mg)2SiO4:Eu, (Ba,Sr,Ca)2(Mg, Zn)Si2O7:Eu, (Ba,Ca,Sr,Mg)9(Sc,Y,Lu,Gd)2(Si,Ge)6O24: Eu, Y2SiO5:CeTb, Sr2P2O7 Sr2B2O5:Eu, Sr2Si3O8—2SrCl2:Eu, Zn2SiO4:Mn, CeMgAl11O19:Tb, Y3Al5O12:Tb, Ca2Y8(SiO4)6O2:Tb, La3Ga5SiO14:Tb, (Sr,Ba,Ca)Ga2S4:Eu,Tb,Sm, Y3(Al,Ga)5O12:Ce, (Y,Ga,Tb,La,Sin,Pr,Lu)3(Al,Ga)5O12:Ce, Ca3Sc2Si3O12:Ce, Ca3(Sc,Mg,Na,Li)2Si3O12:Ce, CaSc2O4:Ce, Eu-activated β-Sialon, SrAl2O4:Eu, (La,Gd,Y)2O2S:Tb, CeLaPO4:Tb, ZnS:Cu,Al, ZnS:Cu,Au,Al, (Y,Ga,Lu,Sc,La)BO3:Ce,Tb, Na2Gd2B2O7:Ce,Tb, (Ba,Sr)2(Ca,Mg,Zn)B2O6:K,Ce,Tb, Ca8Mg (SiO4)4Cl2:Eu,Mn, (Sr,Ca,Ba)(Al,Ga,In)2S4:Eu, (Ca,Sr)8 (Mg,Zn)(SiO4)4Cl2:Eu,Mn, M3Si6O9N4:Eu, Sr5Al5Si21O2N35:Eu, Sr3Si13Al3N21O2:Eu, (Mg,Ca,Sr,Ba)2Si5N8:Eu, (La,Y)2O2S:Eu, (Y,La,Gd,Lu)2O2S:Eu, Y(V,P)O4:Eu, (Ba,Mg)2SiO4:Eu,Mn, (Ba,Sr,Ca,Mg)2SiO4:Eu,Mn, LiW2O8:Eu, LiW2O8:Eu,Sm, Eu2W2O9, Eu2W2O9:Nb and Eu2W2O9:Sm, (Ca,Sr)S:Eu, YAlO3:Eu, Ca2Y8(SiO4)6O2:Eu, LiY9(SiO4)6O2:Eu, (Y,Gd)3Al5O12:Ce, (Tb,Gd)3Al5O12:Ce, (Mg,Ca,Sr,Ba)2Si5(N,O)8:Eu, (Mg,Ca,Sr,Ba)Si(N,O)2:Eu, (Mg,Ca,Sr,Ba)AlSi(N,O)3:Eu, (Sr,Ca,Ba,Mg)10(PO4)6Cl2:Eu, Mn, Eu,Ba3MgSi2O8:Eu,Mn, (Ba,Sr,Ca,Mg)3(Zn,Mg)Si2O8:Eu,Mn, (k-x)MgO·xAF2·GeO2:yMn4+ (wherein k=2.8 to 5, x=0.1 to 0.7, y=0.5 to 0.015, A=Ca, Sr, Ba, Zn or a mixture thereof), Eu-activated α-Sialon, (Gd,Y,Lu,La)2O3:Eu, Bi, (Gd,Y,Lu,La)2O2S:Eu,Bi, (Gd,Y, Lu,La)VO4:Eu,Bi, SrY2S4:Eu,Ce, CaLa2S4:Ce,Eu, (Ba,Sr,Ca)MgP2O7:Eu, Mn, (Sr,Ca,Ba,Mg,Zn)2P2O7:Eu,Mn, (Y,Lu)2WO6:Eu,Ma, (Ba,Sr,Ca)xSiyNz:Eu,Ce (wherein x, y and z are integers equal to or greater than 1),(Ca,Sr,Ba,Mg)10(PO4)6(F,Cl,Br,OH):Eu,Mn, ((Y,Lu,Gd,Tb)1-x-yScxCey)2(Ca,Mg)(Mg,Zn)2+rSiz-qGeqO12+δ, SrAlSi4N7, Sr2Al2Si9O2N14:Eu, M1aM2bM3cOd (wherein M1=activator element including at least Ce, M2=bivalent metal element, MS trivalent metal element, 0.1≤a≤0.2, 0.8≤b≤2, 1.6c2.4 and 3.2≤d≤4.8), A2+xMyMnzFn (wherein A=Na and/or K; M=Si and Al, and −1≤x≤1, 0.9≤y+z≤1.1, 0.1≤z≤0.4 and 5≤n≤7), KSF/KSNAF, or (Lai Eux, Lny)2O2S (wherein 0.02≤x≤0.50 and 0≤y≤0.50, Ln=Y3+, Gd3+, Lu3+, Sc3+, Sm3+ or Er 3+). In some preferred embodiments, the luminescent materials may comprise phosphors comprising one or more of the following materials: CaAlSiN3:Eu, (Sr,Ca)AlSiN3:Eu, BaMgAl10O17:Eu, (Ba,Ca,Sr,Mg)2SiO4:Eu, β-SiAlON, Lu3Al5O12:Ce, Eu3+(Cd0.9Y0.1)3Al5O12:Bi3+, Tb3+, Y3Al5O12:Ce, La3Si6N11:Ce, (La,Y)3Si6N11:Ce, Ca2AlSi3O2N5:Ce3+, Ca2AlSi3O2N5:Ce3+,Eu2+, Ca2AlSi3O2N5:Eu2+, BaMgAl10O17:Eu2+, Sr4.5Eu0.5(PO4)3Cl, or M1aM2bM3cOd (wherein M1=activator element comprising Ce, M2=bivalent metal element, M3=trivalent metal element, 2, and 3.2≤d≤4.8). In further preferred embodiments, the luminescent materials may comprise phosphors comprising one or more of the following materials: CaAlSiN3:Eu, BaMgAl10O17:Eu, Lu3Al5O12:Ce, or Y3Al5O12:Ce.

In certain embodiments, the luminophoric mediums can include luminescent materials that comprise one or more quantum materials. Throughout this specification, the term “quantum material” means any luminescent material that includes: a quantum dot; a quantum wire; or a quantum well. Some quantum materials may absorb and emit light at spectral power distributions having narrow wavelength ranges, for example, wavelength ranges having spectral widths being within ranges of between about 25 nanometers and about 50 nanometers. In examples, two or more different quantum materials may be included in a lumiphor, such that each of the quantum materials may have a spectral power distribution for light emissions that may not overlap with a spectral power distribution for light absorption of any of the one or more other quantum materials. In these examples, cross-absorption of light emissions among the quantum materials of the lumiphor may be minimized. Throughout this specification, the term “quantum dot” means: a nanocrystal made of semiconductor materials that are small enough to exhibit quantum mechanical properties, such that its excitons are confined in all three spatial dimensions. Throughout this specification, the term “quantum wire” means: an electrically conducting wire in which quantum effects influence the transport properties. Throughout this specification, the term “quantum well” means: a thin layer that can confine (quasi-)particles (typically electrons or holes) in the dimension perpendicular to the layer surface, whereas the movement in the other dimensions is not restricted.

Some embodiments of the present invention relate to use of solid state emitter packages. A solid state emitter package typically includes at least one solid state emitter chip that is enclosed with packaging elements to provide environmental and/or mechanical protection, color selection, and light focusing, as well as electrical leads, contacts or traces enabling electrical connection to an external circuit. Encapsulant material, optionally including luminophoric material, may be disposed over solid state emitters in a solid state emitter package. Multiple solid state emitters may be provided in a single package. A package including multiple solid state emitters may include at least one of the following: a single leadframe arranged to conduct power to the solid state emitters, a single reflector arranged to reflect at least a portion of light emanating from each solid state emitter, a single submount supporting each solid state emitter, and a single lens arranged to transmit at least a portion of light emanating from each solid state emitter.

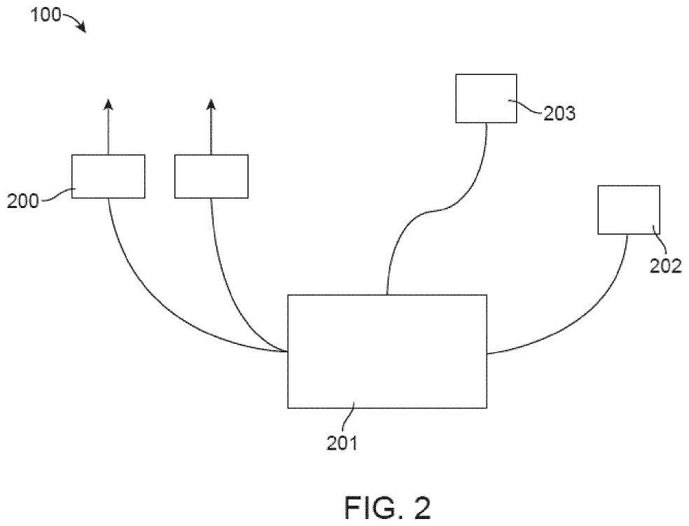

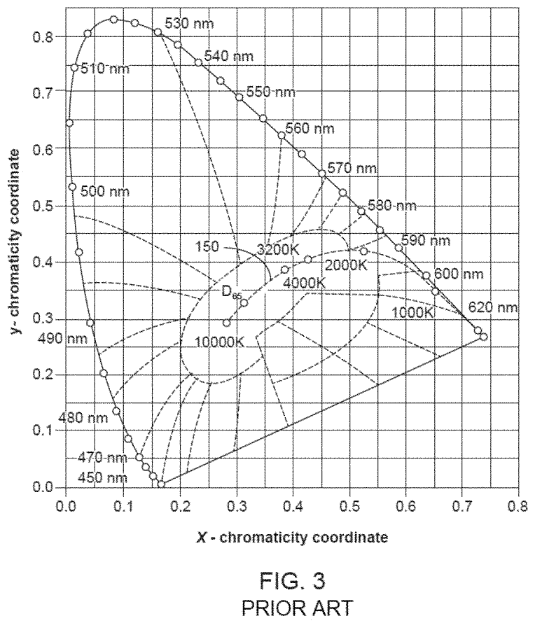

Individual LEDs or groups of LEDs in a solid state package wired in series) may be separately controlled. As depicted schematically in FIG. 2, multiple solid state packages 200 may be arranged in a single semiconductor light emitting device 100. Individual solid state emitter packages or groups of solid state emitter packages (e.g., wired in series) may be separately controlled. Separate control of individual emitters, groups of emitters, individual packages, or groups of packages, may be provided by independently applying drive currents to the relevant components with control elements known to those skilled in the art. In one embodiment, at least one control circuit 201 may comprise a multichannel driver having a current supply circuit configured to independently apply an on-state drive current to each individual solid state emitter, group of solid state emitters, individual solid state emitter package, or group of solid state emitter packages. Such control may be responsive to a control signal (optionally including at least one sensor 202 arranged to sense electrical, optical, and/or thermal properties and/or environmental conditions), and a control system 203 may be configured to selectively provide one or more control signals to the at least one current supply circuit. The design and fabrication of semiconductor light emitting devices are well known to those skilled in the art, and hence further description thereof will be omitted. In various embodiments, current to different circuits or circuit portions may be pre-set, user-defined, or responsive to one or more inputs or other control parameters. The lighting systems can be controlled via methods described in U.S. Provisional Patent Application Ser. No. 62/491,137, filed Apr. 27, 2017, entitled Methods and Systems for An Automated Design, Fulfillment, Deployment and Operation Platform for Lighting Installations, United States Provisional Patent Application Ser. No. 62/562,714, filed Sep. 25, 2017, entitled Methods and Systems for An Automated Design, Fulfillment, Deployment and Operation SO Platform for Lighting Installations, and International Patent Application No. PCT/US2018/029380, filed Apr. 25, 2018 and entitled Methods and Systems for an Automated Design, Fulfillment, Deployment and Operation Platform for Lighting Installations, published as International Publication No. WO 2018/2685 A2, each of which hereby are incorporated by reference as if fully set forth herein in their entirety.

In some embodiments, the present disclosure provides semiconductor light emitting devices 100 that include a plurality of LED strings, with each LED string having a recipient luminophoric medium that comprises a luminescent material. In some embodiments, different combinations of lighting channels 105A-F can be present in the lighting systems of the present disclosure. Each lighting channel 105A-F can emit light at a particular color point on the 1931 CIE Chromaticity Diagram and with particular spectral power characteristics. By utilizing different combinations of lighting channels, different operational modes can be provided that can provide tunable white light between particular CCT values and with particular characteristics.

In some embodiments, the different operational modes can provide for substantially different circadian-stimulating energy characteristics. A first LED string 101A and a first luminophoric medium 102A together can emit a first light having a first color point within a blue color range. The combination of the first LED string 101A and the first luminophoric medium 102A are also referred to herein as a “blue channel” 105A. A second LED string 101B and a second luminophoric medium 102B together can emit a second light having a second color point within a red color range. The combination of the second LED string 101A and the second luminophoric medium 102A are also referred to herein as a “red channel” 105B. A third LED string 1010 and a third luminophoric medium 102C together can emit a third light having a third color point within a short-blue-pumped cyan color range. The combination of the third LED string 101C and the third luminophoric medium 102C are also referred to herein as a “short-blue-pumped cyan channel” or “SBC channel” 105C. A fourth LED string 101D and a fourth luminophoric medium 102D together can emit a fourth light having a fourth color point within a long-blue-pumped cyan color range. The combination of the fourth LED string 101D and the fourth luminophoric medium 102D are also referred to herein as a “long-blue-pumped cyan channel” or “LBC channel” 105D. A fifth LED string 101E and a fifth luminophoric medium 102E together than emit a fifth light having a fifth color point within a yellow color range. The combination of the fifth LED string 101E and the fifth luminophoric medium 102E are also referred to herein as a “yellow channel” 105E. A sixth LED string 101E and a sixth luminophoric medium 102F together than emit a sixth light having SO a fifth color point within a violet color range. The combination of the sixth LED string 101F and the sixth luminophoric medium 102F are also referred to herein as a “violet channel” 105F. It should be understood that the use of the terms “blue”, “red”, “cyan”, “yellow”, and “violet” for the color ranges and channels are not meant to be limiting in terms of actual color outputs, but are used as a naming convention herein, as those of skill in the art will appreciate that color points within color ranges on the 1931 CIE Chromaticity Diagram for the channels may not have the visual appearance of what may commonly be referred to as “blue” “red”, “cyan”, “yellow”, and “violet” by laymen, and may have the appearance of other colored light or white or near-white light, for example, in some embodiments.

The first, second, third, fourth, fifth, and sixth LED strings 101A-F can be provided with independently applied on-state drive currents in order to tune the intensity of the first, second, third, and fourth unsaturated light produced by each string and luminophoric medium together. By varying the drive currents in a controlled manner, the color coordinate (ccx, ccyt of the total light that is emitted from the device 100 can be tuned.

In some embodiments, the device 100 can provide light at substantially the same color coordinate with different spectral power distribution profiles, which can result in different light characteristics at the same CCT. In some embodiments, white light can be generated in modes that produce light from different combinations of one, two, three, or four of the LED strings 101A-F. In some embodiments, white light is generated using only the first, second, and third LED strings, i.e. the blue, red, and short-blue-pumped cyan channels, referred to herein as “high-CM mode”. In other embodiments, white light is generated using the first, second, third, and fourth LED strings, i.e., the blue, red, short-blue-pumped cyan, and long-blue-pumped cyan channels, in what is referred to herein as a “highest-CRI mode”. In further embodiments, white light can be generated using the first, second, and fourth LED strings, i.e. the blue, red, and long-blue-pumped cyan channels, in what is referred to herein as a “high-EML mode”.

In other embodiments, white light can be generated using the first, second, fifth, and sixth LED strings, i.e. the blue, red, yellow, and violet channels, in what is also referred to herein as a “low-EML mode”. In yet further embodiments, white light can be generated using the second, fifth, and sixth LED strings, i.e. the red, yellow, and violet channels, in what is also referred to herein as a “very-low-EML mode”.

In certain embodiments, switching between the high-CRI mode and the high EML mode can increase the EML by about 5%, about 10%, about 15%, about 20%, about 25%, about 30%, about 35%, about 40%, about 45%, about 50%, about 55%, about 60%, about 65%, about 70%, about 75%, about 80%, or about 85% while providing a Ra value within about 1, about 2, about 3, about 4, about 5, about 6, about 7, about 8, about 9, or about 10 while generating white light at substantially the same color point on the 1931 Chromaticity Diagram. In some embodiments, the light generated in two operating modes being switched between can produce white light outputs that can be within about 1.0 standard deviations of color matching (SDCM). In some embodiments, the light generated in two operating modes being switched between can produce white light outputs that can be within about 0.5 standard deviations of color matching (SDCM). In some embodiments the methods can further comprise switching among two or more of the first and second operating modes while sequentially generating white light at a plurality of color points within a 7-step MacAdam ellipse of points on the black body locus having a correlated color temperature between 1800 K and 1000 K. In certain embodiments the methods further comprise switching between operating modes while tuning the light that is generated between color points of different correlated color temperatures.

In certain embodiments, switching between the high-CRI mode and high-EVIL or very-low EML mode can reduce EMIL by about 5%, about 10%, about 15%, about 20%, about 25%, about 30%, about 35%, about 40%, about 45%, about 50%, about 55%, about 60%, about 65%, about 70%, about 75%, about 80%, or about 85% while providing a Ra value within about 1, about 2, about 3, about 4, about 5, about 6, about 7, about 8, about 9, or up to about 20 while generating white light at substantially the same color point on the 1931 Chromaticity

Diagram. In some embodiments, the light generated in two operating modes being switched between can produce white light outputs that can be within about 1.0 standard deviations of color matching (SDCM). In some embodiments, the light generated in two operating modes being switched between can produce white light outputs that can be within about 0.5 standard deviations of color matching (SDCM). In some embodiments the methods can further comprise switching among two or more of the first and second operating modes while sequentially generating white light at a plurality of color points within a 7-step MacAdam ellipse of points on the black body locus having a correlated color temperature between 1800 K and 10000 K. In certain embodiments the methods further comprise switching between operating modes while tuning the light that is generated between color points of different correlated color temperatures.

In some embodiments, only two of the LED stings are producing light during the generation of white light in any one of the operational modes described herein, as the other two LED strings are not necessary to generate white light at the desired color point with the desired color rendering performance. In certain embodiments, substantially the same color coordinate (ccx, ccy) of total light emitted from the device can be provided in two different operational modes (different combinations of two or more of the channels), but with different color-rendering, circadian, or other performance metrics, such that the functional characteristics of the generated light can he selected as desired by users.

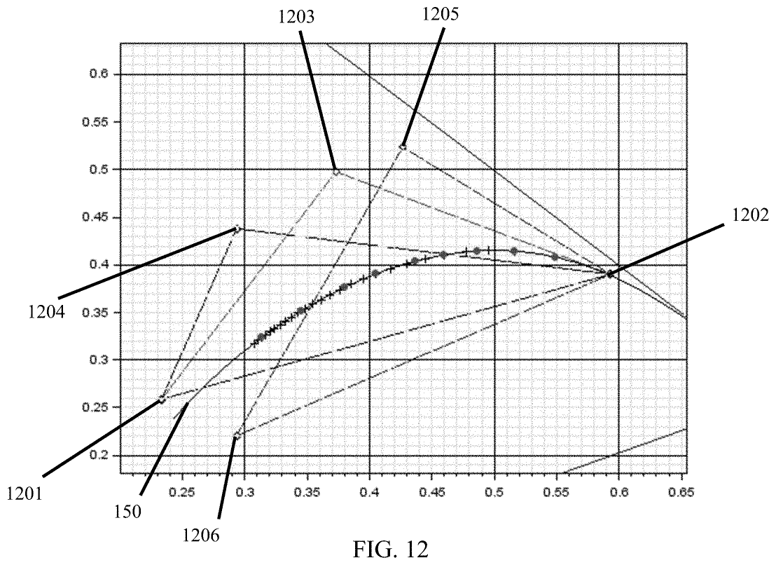

Non-limiting FIG. 12 shows a portion of the 1931 CIE Chromaticity Diagram with Planckian locus 150 and some exemplary color points and triangles connecting color points to depict the tunable gamut of color points from various combinations of lighting channels. FIG. 12 shows an exemplary first color point 1201 produced from a blue channel, an exemplary second color point 1202 produced from a red channel, an exemplary third color point 1203 produced from a short-blue-pumped cyan channel, an exemplary fourth color point 1204 produced from a long-blue-pumped cyan channel, an exemplary fifth color point 1205 produced from a yellow channel, and an exemplary sixth color point 1206 produced from a violet channel. In other embodiments, the color points 1201, 1202, 1203, 1204, 1205, and 1206 may fall at other (ccx, ccy) coordinates within suitable color ranges for each lighting channel as describe more fully below.

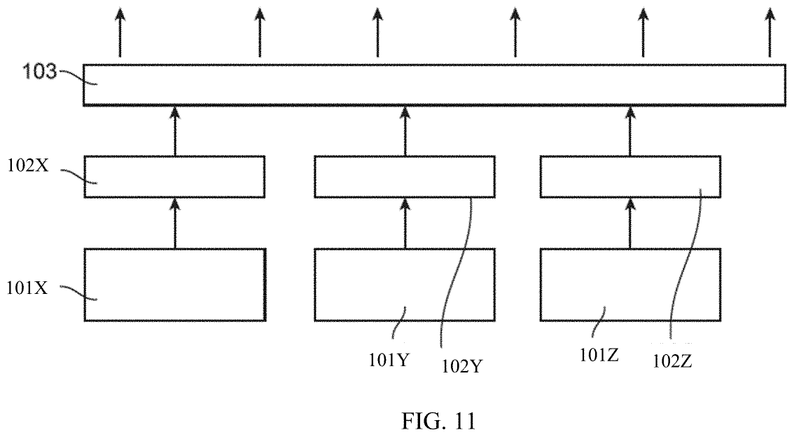

In some embodiments, the semiconductor light emitting devices of the disclosure can comprise only three, four, or five of the lighting channels described herein. FIG. 11 illustrates a device 100 having only three LED strings 101X/101Y/101Z with associated luminophoric mediums 102X/102Y/102Z. The three channels depicted can be any combination of three of lighting channels described elsewhere throughout this disclosure. In some embodiments, red, blue, and long-blue-pumped cyan channels are provided. In other embodiments, red, blue, and short-blue-pumped cyan channels are provided. In other embodiments, red, short-blue-pumped cyan, and long-blue-pumped cyan channels are provided. In yet other embodiments, blue, short-blue-pumped cyan, and long-blue-pumped cyan channels are provided. In further embodiments, red, yellow, and violet channels are provided. In further embodiments, one of the three, four, or five different channels of a lighting system can be duplicated as an additional channel, so that four, five, or six channels are provided, but two of the channels are duplicates of each other.

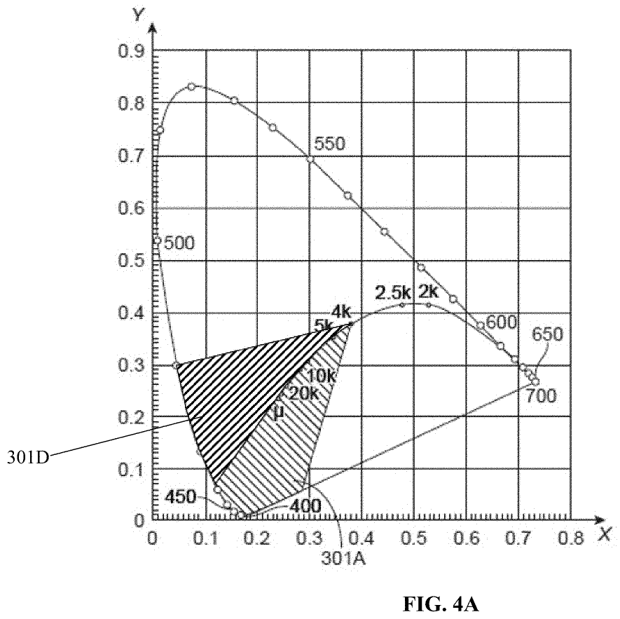

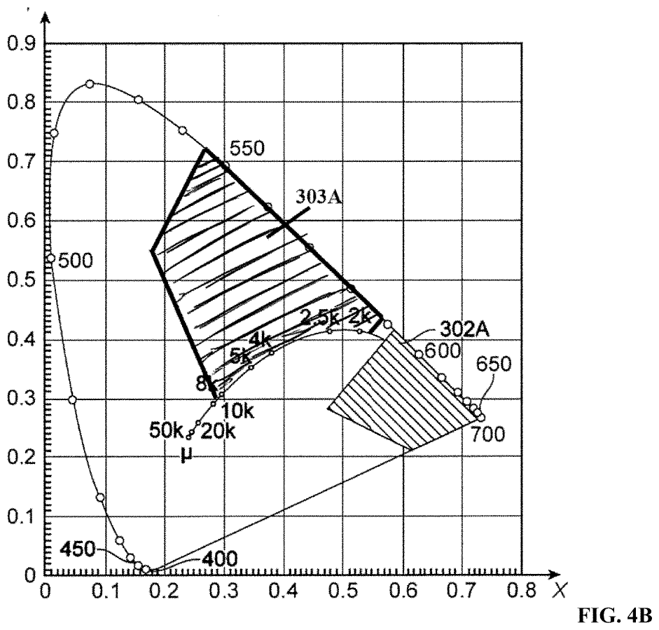

FIGS. 4A, 4B, 5-10, 13, 14A, and 14B depict suitable color ranges for some embodiments of the disclosure as described in more detail elsewhere herein. It should be understood that any gaps or openings in the described boundaries for the color ranges should be closed with straight lines to connect adjacent endpoints in order to define a closed boundary for each color range.

In some embodiments of the present disclosure, lighting systems can include blue channels that produce light with a blue color point that falls within a blue color range. In certain embodiments, suitable blue color ranges can include blue color ranges 301A-F. FIG. 4A depicts a blue color range 301A defined by a line connecting the ccx, ccy color coordinates of the infinity point of the Planckian locus (0.242, 0.24) and (0.12, 0.068), the Planckian locus from 4000 K and infinite CCT, the constant CCT line of 4000 K, the line of purples, and the spectral locus,

FIG. 4A also depicts a blue color range 301D defined by a line connecting (0.3806, 0.3768) and (0.0445, 0.3), the spectral locus between the monochromatic point of 490 nm and (0.12, 0.068), a line connecting the ccx, ccy color coordinates of the infinity point of the Planckian locus (0.242, 0.24) and (0.12., 0.068), and the Planckian locus from 4000 K and infinite CCT. The blue color range may also be the combination of ranges 301A and 301D together.

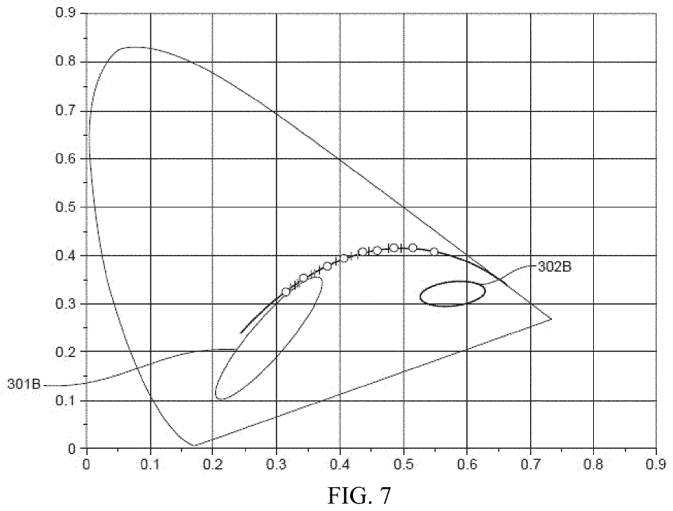

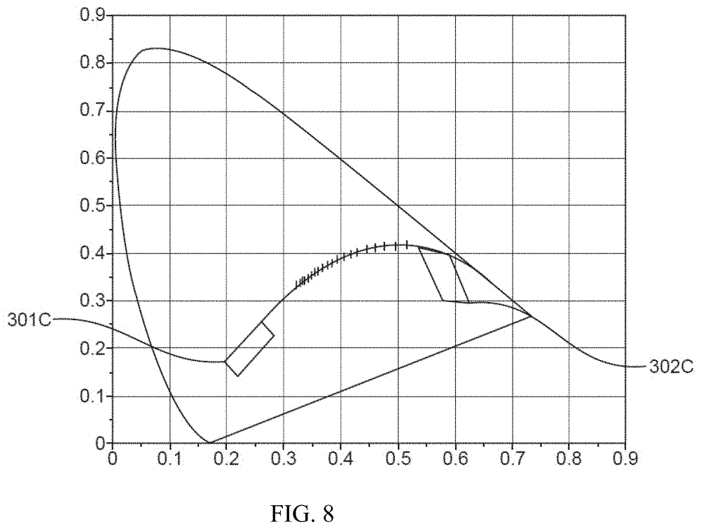

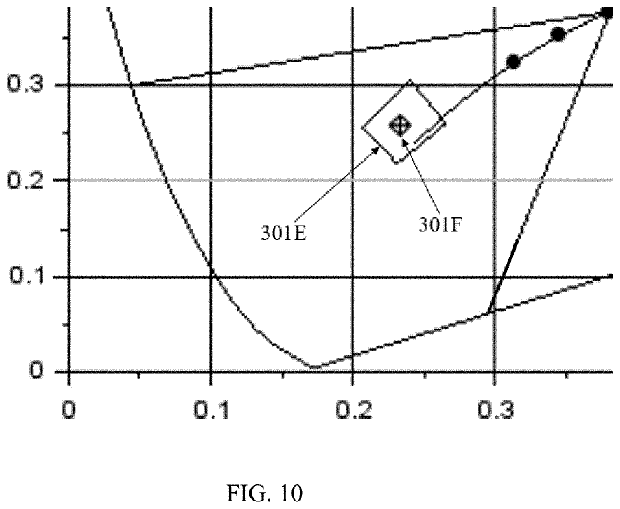

FIG. 7 depicts a blue color range 301B can be defined by a 60-step MacAdam ellipse at a CCT of 2000 K, 40 points below the Planckian locus. FIG. 8 depicts a blue color range 301C that is defined by a polygonal region on the 1931 CIE Chromaticity Diagram defined by the following ccx, ccy color coordinates: (0.22, 0:14), (0.19, 0.17), (0.26, 0.26), (0.28, 0.23), FIG. 10 depicts blue color ranges 301E and 301F. Blue color range 301E is defined by lines connecting (0.231, 0.218), (0.265, 0.260), (0.2405, 0.305), and (0.207, 0.256).

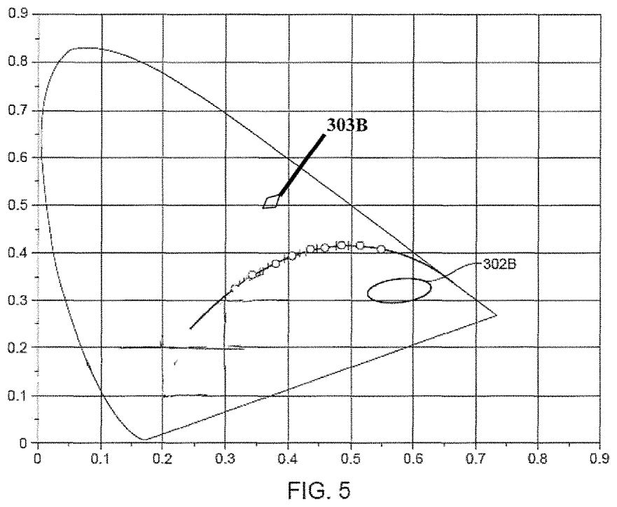

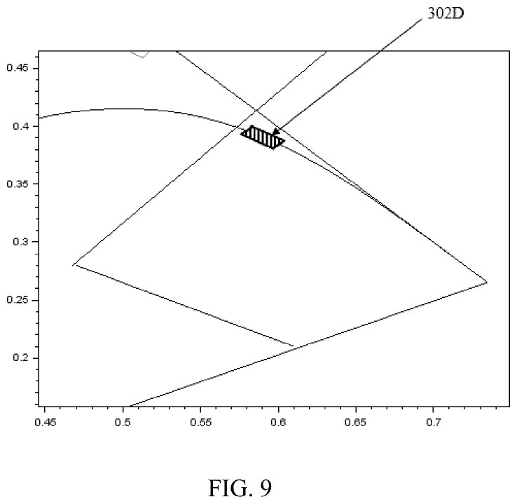

In some embodiments of the present disclosure, lighting systems can include red channels that produce light with a red color point, that falls within a red color range. In certain embodiments, suitable red color ranges can include red color ranges 302A-D. FIG. 4B depicts a red color range 302A defined by the spectral locus between the constant CCT line of 1600 K and the line of purples, the line of purples, a line connecting the ccx, ccy color coordinates (0.61, 0.21) and (0.47, 0.28), and the constant CCT line of 1600 K. FIG. 5 depicts some suitable color ranges for some embodiments of the disclosure. A red color range 302B can be defined by a 20-step MacAdam ellipse at a CCT of 1200 K, 20 points below the Planckian locus. HG. 6 depicts some further color ranges suitable for some embodiments of the disclosure. A red color range 302C is defined by a polygonal region on the 1931 CIE Chromaticity Diagram defined by the following ccx, ccy color coordinates: (0.53, 0.41), (0.59, 0.39), (0.63, 0.29), (0.58, 0,30). In FIG. 8, a red color range 302C is depicted and can be defined by a polygonal region on the 1931 CIF Chromaticity Diagram defined by the following ccx, ccy color coordinates: (0.53, 0.41), (0.59, 0.39), (0.63, 0.29), (0.58, 0.30). FIG. 9 depicts a red color range 302D defined by lines connecting the ccx, ccy coordinates (0.576, 0.393), (0.583, 0.4), (0.604, 0.387), and (0.597, 0.380).

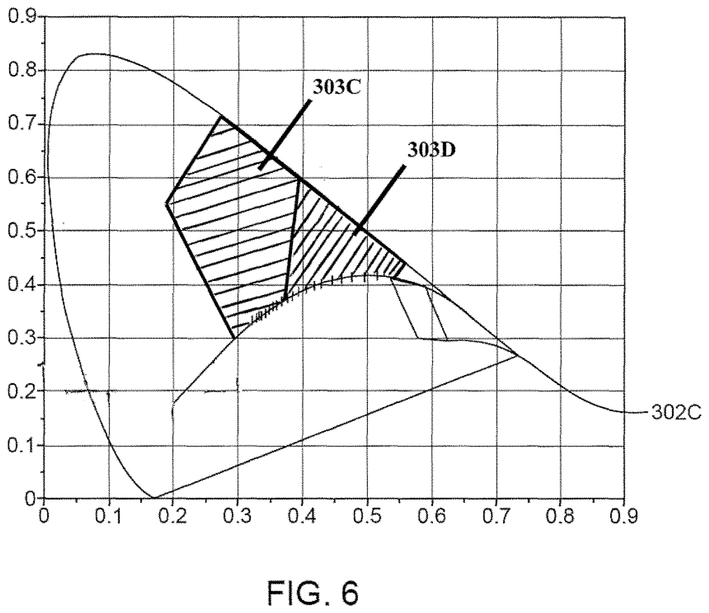

In some embodiments of the present disclosure, lighting systems can include short-blue-pumped cyan channels that produce light with a cyan color point that falls within a cyan color range. In certain embodiments, suitable cyan color ranges can include cyan color ranges 303A-D. FIG. 4B shows a cyan color range 303A defined by a line connecting the ccx, ccy color coordinates (0.18, 0.55) and (0.27, 0.72), the constant CCT line of 9000 K, the Planckian locus between 9000 K and 1800 K, the constant CCT line of 1800 K, and the spectral locus. FIG. 5 depicts some suitable color ranges for some embodiments of the disclosure. A cyan color range 303B can be defined by the region bounded by lines connecting (0.360, 0.495), (0.371, 0.518), (0.388, 0.522), and (0.377, 0.499). FIG. 6 depicts some further color ranges suitable for some embodiments of the disclosure. A cyan color range 303C is defined by a line connecting the ccx, ccy color coordinates (0.18, 0,55) and (0.27, 0.72), the constant CCT line of 9000 K, the Planckian locus between 9000 K and 4600 K, the constant CCT line of 4600 K, and the spectral locus. A cyan color range 303D is defined by the constant CCT line of 4600 K, the spectral locus, the constant CCT line of 1800 K, and the Planckian locus between 4600 K. and 1800 K.

In some embodiments of the present disclosure, lighting systems can include long-blue-pumped cyan channels that produce light with a cyan color point that falls within a cyan color range. In certain embodiments, suitable cyan color ranges can include cyan color ranges 303A-E. FIG. 4B shows a cyan color range 303A defined by a line connecting the ccx, ccy color coordinates (0.18, 0.55) and (0.27, 0.72), the constant CCT line of 9000 K, the

Planckian locus between 90001 K and 1800 K, the constant CCT line of 1800 K, and the spectral locus. FIG. 5 depicts some suitable color ranges for some embodiments of the disclosure. A cyan color range 303B can be defined by the region bounded by lines connecting (0.360, 0.495), (0.371, 0.518), (0.388, 0.522), and (0.377, 0.499). FIG. 6 depicts some further color ranges suitable for some embodiments of the disclosure. A cyan color range 303C is defined by a line connecting the ccx, ccy color coordinates (0.18, 0.55) and (0.2.7, 0.72), the constant CCT line of 9000 K, the Planckian locus between 9000 K and 4600 K, the constant CCT line of 4600 K, and the spectral locus. A cyan color range 303D is defined by the constant CCI line of 4600 K, the spectral locus, the constant CCT line of 1800 K, and the Planckian locus between 4600 K and 1800 K. In some embodiments, the long-blue-pumped cyan channel can provide a color point within a cyan color region 303E defined by lines connecting (0.497, 0.469), (0.508, 0.484), (0.524, 0.472), and (0.513, 0.459).

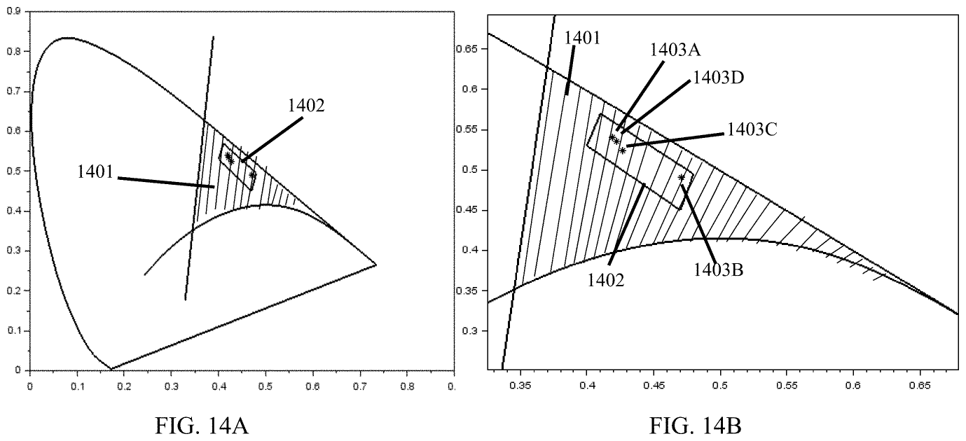

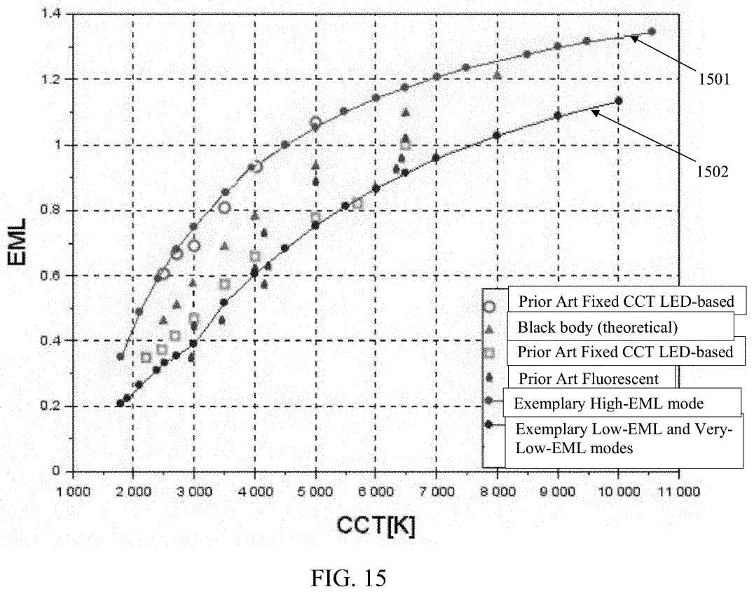

In some embodiments of the present disclosure, lighting systems can include yellow channels that produce light with a yellow color point that falls within a yellow color range. Non-limiting FIGS. 14A and 14B depicts some aspects of suitable yellow color ranges for embodiments of yellow channels of the present disclosure. In some embodiments, the yellow channels can produce light having a yellow color point that falls within a yellow color range 1401, with boundaries defined on the 1931 CIE Chromaticity Diagram of the constant CCI line of 5000 K from the Planckian locus to the spectral locus, the spectral locus, and the Planckian locus from 5000 K to 5500 K. In certain embodiments, the yellow channels can produce light having a yellow color point that falls within a yellow color range 1402, with boundaries defined on the 1931 CIE Chromaticity Diagram by a polygon connecting (ccx, ccy) coordinates of (0.47, 0.45), (0.48, 0.495), (0.41, 0.57), and (0.40, 0.53). In some embodiments, the yellow channels can produce light having a color point at one of the exemplary yellow color points 1403A-D shown in FIG. 14 and described more fully elsewhere herein.

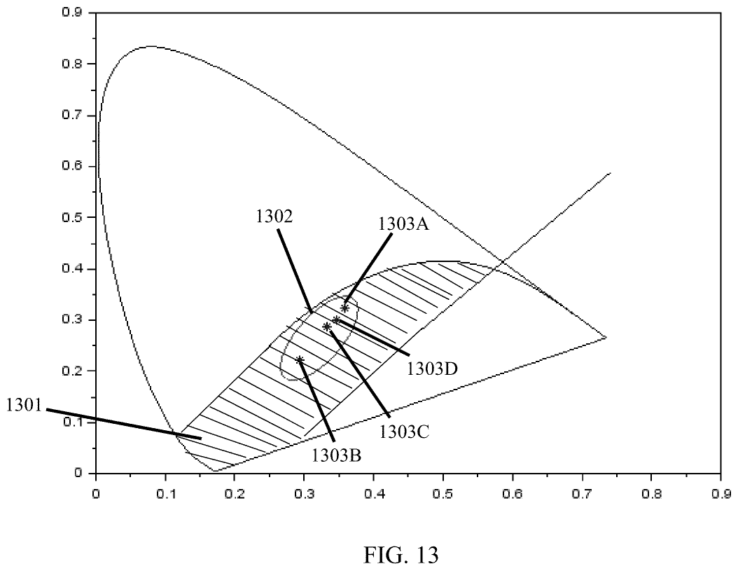

In some embodiments of the present disclosure, lighting systems can include violet channels that produce light with a violet color point that falls within a violet color range. Non-limiting FIG. 13 depicts some aspects of suitable violet color ranges for embodiments of violet channels of the present disclosure. In some embodiments, the violet channels can produce light having a violet color point that falls within a violet color range 1301, with boundaries defined on the 1931 CIE Chromaticity Diagram of the Planckian locus between 1600 K CCT and infinite CCT, a line between the infinite CCT point on the Planckian locus and the monochromatic point of 470 nm on the spectral locus, the spectral locus between the monochromatic point of 470 nm and the line of purples, the line of purples from the spectral locus to the constant CCT line of 1600 K, and the constant CCT line of 1600 K between the line of purples and the 1600 K CCT point on the Planckian locus. In certain embodiments, the violet channels can produce light having a violet color point that falls within a violet color range 1302, with boundaries defined on the 1931 CIE Chromaticity Diagram by a 40-step MacAdam ellipse centered at 6500 K CCT with DUV=−40 points. In some embodiments, the violet channels can produce light having a color point at one of the exemplary violet color points 1303A-D shown in FIG. 13 and described more fully elsewhere herein.

In some embodiments, the LEDs in the first, second, third and fourth LED strings can be LEDs with peak emission wavelengths at or below about 535 nm. In some embodiments, the LEDs emit light with peak emission wavelengths between about 360 nm and about 535 nm. In some embodiments, the LEDs in the first, second, third and fourth LED strings can be formed from InGaN semiconductor materials. In some preferred embodiments, the first, second, and third LED strings can have LEDs having a peak wavelength between about 405 nm and about 485 nm, between about 430 nm and about 460 nm, between about 430 nm and about 455 nm, between about 430 nm and about 440 nm, between about 440 am and about 450 nm, between about 440 nm and about 445 nm, or between about 445 nm and about 450 nm. The LEDs used in the first, second, third, and fourth LED strings may have full-width half-maximum wavelength ranges of between about 10 nm and about 30 nm. In some preferred embodiments, the first, second, and third LED strings can include one or more LUXEON Z Color Line royal blue LEDs (product code LXZ1-PR01) of color bin codes 3, 4, 5, or 6, one or more LUXEON Z Color Line blue LEDs (LXZ1-PB01) of color bin code 1 or 2, or one or more LUXEON royal blue LEDs (product code LXML-PR01 and LXML-PR02) of color bins 3, 4, 5, or 6 (Lumileds Holding B.V., Amsterdam, Netherlands).

In some embodiments, the LEDs used in the fourth LED string can be LEDs having peak emission wavelengths between about 360 nm and about 535 nm, between about 380 nm and about 520 nm, between about 470 nm and about 505 nm, about 480 nm, about 470 nm, about 460 nm, about 455 nm, about 450 nm, or about 445 nm. In certain embodiments, the LEDs used in the fourth LED string can have a peak wavelength between about 460 nm and 515 nm. In some embodiments, the LEDs in the fourth LED string can include one or more LUXEON Rebel Blue LEDs (LXML-PB01, LXML-PB02) of color bins 1, 3, 4, or 5, which have peak wavelengths ranging from 460 nm to 485 nm, or LUXEON Rebel Cyan LEDs (LXML-PE01) of color bins 1, 2, 3, 4, or 5, which have peak wavelengths raving from 460 nm to 485 nm.

In certain embodiments, the LEDs used in the fifth and sixth LED strings can be LEDs having peak wavelengths of between about 380 nm and about 420 nm, such as one or more LEDs having peak wavelengths of about 380 nm, about 385 nm, about 390 nm, about 395 nm, about 4 nm, about 405 nm, about 410 nm, about 415 nm, or about 420 nm. In some embodiments, the LEDs in the fifth and sixth LED strings can be one or more LUXEON ZUV LEDs (product codes LHUV-0380-, LHUV-0385-, LHUV-0390-, LHUV-0395-, LHUV-04-, LHUV-0405-, LHUV-0410-, LI-EUV-0415-, LHUV-0420-,) (Lumileds Holding B.V., Amsterdam, Netherlands), one or more LUXEON UV FC LEDs (product codes LxF3-U410) (Lumileds Holding B.V., Amsterdam, Netherlands), one or more LUXEON UV U LEDs (product code LHUV-0415-) (Lumileds Holding B.V., Amsterdam, Netherlands), for example. Similar LEDs to those described herein from other manufacturers such as OSRAM GmbH and Cree, Inc. could also be used, provided they have peak emission and full-width half-maximum wavelengths of the appropriate values.

In embodiments utilizing :LEDs that emit substantially saturated light at wavelengths between about 360 nm and about 535 nm, the device 100 can include suitable recipient luminophoric mediums for each LED in order to produce light having color points within the suitable blue color ranges 301A-F, red color ranges 302A-D, cyan color ranges 303A-E, violet color ranges 1301, 1302, and yellow color ranges 1401, 1402 described herein. The light emitted by each lighting channel (from each LED string, i.e., the light emitted from the LED(s) and associated recipient luminophoric medium together) can have a suitable spectral power distribution (“SPD”) having spectral power with ratios of power across the visible wavelength spectrum from about 380 nm to about 780 nm or across the visible and near-visible wavelength spectrum from about 320 nm to about 8 nm. While not wishing to be hound by any particular theory, it is speculated that the use of such LEDs in combination with recipient luminophoric mediums to create unsaturated light within the suitable color ranges 301A-F, 302A-D, 303A-E, 1301, 1302, 1401, and 1402 provides for improved color rendering performance for white light across a predetermined range of CCTs from a single device 100.

Further, while not wishing to be bound by any particular theory, it is speculated that the use of such LEDs in combination with recipient luminophoric mediums to create unsaturated light within the suitable color ranges 301A-F, 302A-D, 303A-E, 1301, 1302, 1401, and 1402 provides for improved light rendering performance, providing higher EML performance along with color-rendering performance, for white light across a predetermined range of CCTs from a single device 100.

Some suitable ranges for spectral power distribution ratios of the lighting channels of the present disclosure are shown in Tables 1-4 and 7-15. The Tables show the ratios of spectral power within wavelength ranges, with an arbitrary reference wavelength range selected for each color range and normalized to a value of 1.0In some embodiments, the lighting channels of the present disclosure can each product a colored light that falls between minimum and maximum values in particular wavelength ranges relative to an arbitrary reference wavelength range. Tables 1, 2, and 7-15 show some exemplary minimum and maximum spectral power values for the blue, red, short-blue-pumped cyan, long-blue-pumped cyan, yellow, and violet channels of the disclosure.

In certain embodiments, the blue lighting channel can produce light with spectral power distribution that falls within the values between Blue minimum 1 and Blue maximum I in the wavelength ranges shown in Table 1, Table 2, or both Tables 1 and 2.

In some embodiments, the red lighting channel can produce light with spectral power distribution that fails within the values between Red minimum 1 and Red maximum I in the wavelength ranges shown in Table 1, Table 2, or both Tables 1 and 2. In some embodiments, the red channel can produce red light having a spectral power distribution that falls within the ranges between the Exemplary Red Channels Minimum and the Exemplary Red Channels Maximum in the wavelength ranges shown in one or more of Tables 7-9.

In some embodiments, the short-blue-pumped cyan can fall within the values between Short-blue-pumped cyan minimum I and Short-blue-pumped cyan maximum 1 in the wavelength ranges shown in Table 1, Table 2, or both Tables 1 and 2. In other embodiments, the short-blue-pumped cyan can fall within the values between Short-blue-pumped cyan minimum 1 and Short-blue-pumped cyan maximum 2 in the wavelength ranges shown in Table 1.

In some embodiments, the Long-Blue-Pumped Cyan lighting channel can produce light with spectral power distribution that falls within the values between Long-Blue-Pumped Cyan minimum 1 and Long-Blue-Pumped Cyan maximum 1 in the wavelength ranges shown in Table 1, Table 2, or both Tables 1 and 2.

In some embodiments, the yellow channel can produce yellow light having a spectral power distribution that falls within the ranges between the Exemplary Yellow Channels Minimum and the Exemplary Yellow Channels Maximum in the wavelength ranges shown in one or more of Tables 13-15.

In some embodiments, the violet channel can produce violet light having a spectral power distribution that falls within the ranges between the Exemplary Violet Channels Minimum and the Exemplary Violet Channels Maximum in the wavelength ranges shown in one or more of Tables 10-12.

While not wishing to be bound by any particular theory, it is speculated that because the spectral power distributions for generated light with color points within the blue, long-blue-pumped cyan, short-blue-pumped cyan, yellow, and violet color ranges contains higher spectral intensity across visible wavelengths as compared to lighting apparatuses and methods that utilize more saturated colors, this allows for improved color rendering for test colors other than R1-R8. International Patent Application No. PCT/1JS2018/020792, filed Mar. 2, 2018, discloses aspects of some additional red, blue, short-pumped-blue (referred to as “green” therein), and long-pumped-blue (referred to as “cyan” therein) channel elements that may be suitable for some embodiments of the present disclosure, the entirety of which is incorporated herein for all purposes.

In some embodiments, the short-blue-pumped cyan channel can produce cyan light having certain spectral power distributions. Tables 3 and 4 show the ratios of spectral power within wavelength ranges, with an arbitrary reference wavelength range selected for the short-blue-pumped cyan color range and normalized to a value of 1.0, for a short-blue-pumped cyan channel that may be used in some embodiments of the disclosure. The exemplary Short-blue-pumped cyan Channel 1 has a ccx, ccy color coordinate shown in Table 5. 1n certain embodiments, the short-blue-pumped cyan channel can have a spectral power distribution with spectral power in one or more of the wavelength ranges other than the reference wavelength range increased or decreased within 30% greater or less, within 20% greater or less, within 10% greater or less, or within 5% greater or less than the values shown in Table 3 or 4. In some embodiments, the long-blue-pumped cyan channel can produce cyan light having certain spectral power distributions. Tables 3 and 4 shows ratios of spectral power within wavelength ranges, with an arbitrary reference wavelength range selected for the long-blue-pumped cyan color range and normalized to a value of 1.0, for several non-limiting embodiments of the long-blue-pumped cyan channel. The exemplary Long-blue-pumped cyan Channel I has a ccx, ccy color coordinate Shown in Table 5. In certain embodiments, the long-blue-pumped cyan channel can have a spectral power distribution with spectral power in one or more of the wavelength ranges other than the reference wavelength range increased or decreased within 30% greater or less, within 20% greater or less, within 10% greater or less, or within 5% greater or less than the values shown in Table 3 and 4.

In some embodiments, the red channel can produce red light having certain spectral power distributions. Tables 3-4 and 7-9 show the ratios of spectral power within wavelength ranges, with an arbitrary reference wavelength range selected for the red color range and normalized to a value of 1.0, for red lighting channels that may be used in some embodiments of the disclosure. The exemplary Red Channel 1 has a ccx, ccy color coordinate of (0.5932, 0.3903). In certain embodiments, the red channel can have a spectral power distribution with spectral power in one or more of the wavelength ranges other than the reference wavelength range increased or decreased within 30% greater or less, within 20% greater or less, within 10% greater or less, or within 5% greater or less than the values shown in Tables 3-4 and 7-9 for Red. Channels 1-11 and the Exemplary Red Channels Average.

In some embodiments, the blue channel can produce blue light having certain spectral power distributions. Tables 3 and 4 show the ratios of spectral power within wavelength ranges, with an arbitrary reference wavelength range selected for the blue color range and normalized to a value of 1.0, for a blue channel that may be used in some embodiments of the disclosure. Exemplary Blue Channel 1 has a ccx, ccv color coordinate of (0.2333, 0.2588). In certain embodiments, the blue channel can have a spectral power distribution with spectral power in one or more of the wavelength ranges other than the reference wavelength range increased or decreased within 30% greater or less, within 20% greater or less, within 10% greater or less, or within 5% greater or less than the values shown in Tables 3 and 4.

In some embodiments, the yellow channel can have certain spectral power distributions. Tables 13-15 show the ratios of spectral power within wavelength ranges, with an arbitrary reference wavelength range selected and normalized to a value of 1.0 for exemplary yellow lighting channels, Yellow Channels 1-6, Table 5 shows some aspects of the exemplary yellow lighting channels for some embodiments of the disclosure. In certain embodiments, the yellow channel can have a spectral power distribution with spectral power in one or more of the wavelength ranges other than the reference wavelength range increased or decreased within 30% greater or less, within 20% greater or less, within 10% greater or less, or within 5% greater or less than the values shown in one or more of Tables 13-15 for Yellow Channels 1-6 and the Exemplary Yellow Channels Average.

In some embodiments, the violet channel can have certain spectral power distributions. Tables 13-15 show the ratios of spectral power within wavelength ranges, with an arbitrary reference wavelength range selected and normalized to a value of 1.0 for exemplary violet lighting channels, Violet Channels 1-5. Table 5 shows some aspects of the exemplary violet lighting channels for some embodiments of the disclosure. In certain embodiments, the violet channel can have a spectral power distribution with spectral power in one or more of the wavelength ranges other than the reference wavelength range increased or decreased within 30% greater or less, within 20% greater or less, within 10% greater or less, or within 5% greater or less than the values shown in one or more of Tables 12-15 for one or more of Violet Channels 1-6 and the Exemplary Violet Channels Average.

In some embodiments, the lighting channels of the present disclosure can each product a colored light having spectral power distributions having particular characteristics. In certain embodiments, the spectral power distributions of some lighting channels can have peaks, points of relatively higher intensity, and valleys, points of relatively lower intensity that fall within certain wavelength ranges and have certain relative ratios of intensity between them.

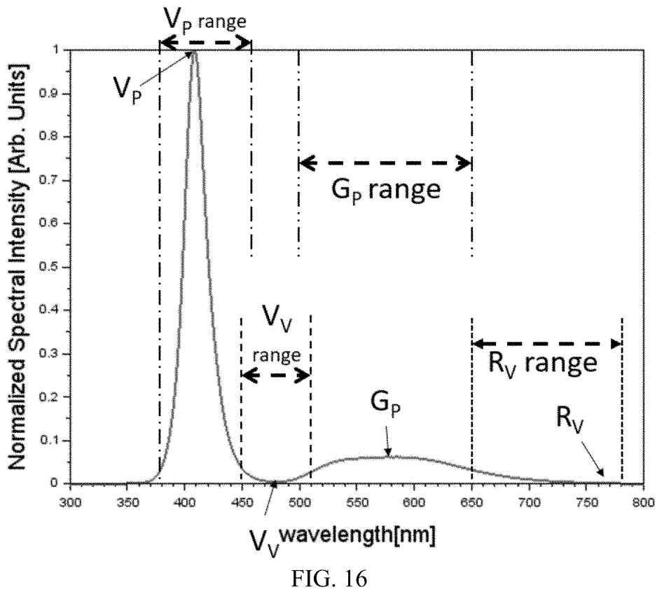

Tables 38 and 39 and FIG. 16 show some aspects of exemplary violet lighting channels for some embodiments of the disclosure. In certain embodiments, a Violet Peak (VP) is present in a range of about 380 nm to about 460 nm. In further embodiments, a Violet Valley (VV) is present in a range of about 450 nm to about 510 nm. In some embodiments, a Green Peak (GP) is present in a range of about 500 nm to about 650 nm. In certain embodiments, a Red Valley (RV) is present in a range of about 650 nm to about 780 nm.

Table 15 shows the relative intensities of the peaks and valleys for exemplary violet lighting channels of the disclosure, with the VP values assigned an arbitrary value of 1.0 in the table. The wavelength at which each peak or valley is present is also shown in Table 15. Table 16 shows the relative ratios of intensity between particular pairs of the peaks and valleys of the spectral power distributions for exemplary violet lighting channels and minimum, average, and maximum values thereof. In certain embodiments, the violet channel can have a spectral power distribution with the relative intensities of VV, GP, and RV increased or decreased within 30% greater or less, within 20% greater or less, within 10% greater or less, or within 5% greater or less than the values shown in Table 15 for one or more of Violet Channels 1-5 and the Exemplary Violet Channels Average. In some embodiments, the violet channel can produce violet light having a spectral power distribution with peak and valley intensities that fall between the Exemplary Violet Channels Minimum and the Exemplary Violet Channels Maximum shown in Table 15. In further embodiments, the violet channel can produce violet light having a spectral power distribution with relative ratios of intensity between particular pairs of the peak and valley intensities that fall between the Exemplary Violet Channels Minimum and the Exemplary Violet Channels Maximum values shown in Table 16. In certain embodiments, the violet channel can have a spectral power distribution with the relative ratios of intensity between particular pairs of the peak and valley intensities increased or decreased within 30% greater or less, within 20% greater or less, within 10% greater or less, or within 5% greater or less than the relative ratio values shown in Table 16 for one or more of Violet Channels 1-5 and the Exemplary Violet Channels Average.

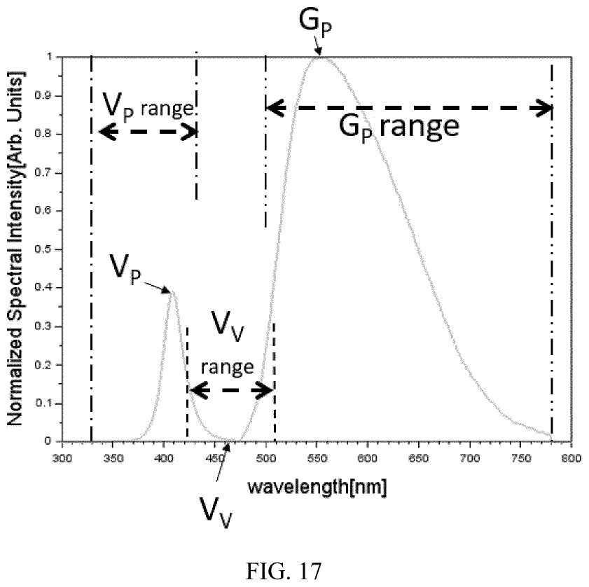

Tables 40 and 41 and FIG. 17 show some aspects of exemplary yellow lighting channels for some embodiments of the disclosure. In certain embodiments, a Violet Peak (VP) is present in a range of about 330 nm to about 430 nm. In further embodiments, a Violet Valley (VV) is present in a range of about 420 nm to about 510 nm. In one embodiment, the yellow channel has a Green Peak (GP) at a wavelength of between about 500 nm and about 780 nm.

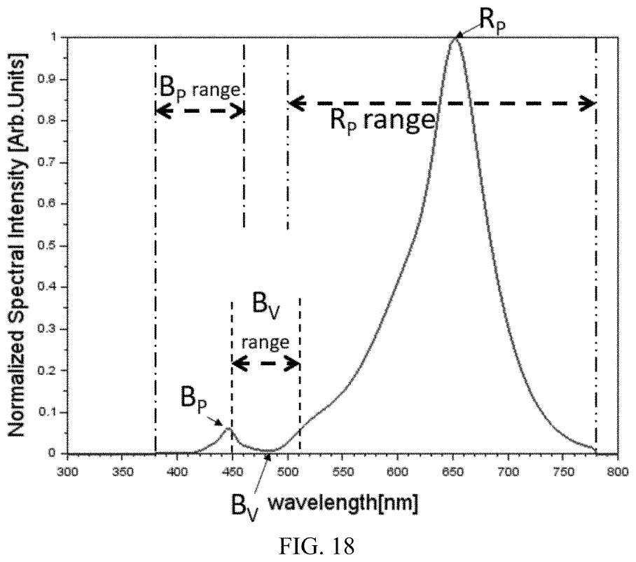

Tables 42, 43, 43A, and 43B and FIG. 18 show some aspects of exemplary red lighting channels for some embodiments of the disclosure. In certain embodiments, a Blue Peak (BP) is present in a range of about 380 nm to about 460 nm. In further embodiments, a Blue Valley (13V) is present in a range of about 450 nm to about 510 nm. In some embodiments, a Red Peak (RP) is present in a range of about 5 nm to about 780 nm. Tables 42 and 43A shows the relative intensities of the peaks and valleys for exemplary red lighting channels of the disclosure, with the RP values assigned an arbitrary value of 1.0 in the table. The wavelength at which each peak or valley is present is also shown in Tables 42 and 43A. Table 20B shows the relative spectral power distributions within particular wavelength ranges, with values relative to the spectral power 470<λ≤510. Table 20 shows the relative ratios of intensity between particular pairs of the peaks and valleys of the spectral power distributions for exemplary red lighting channels and minimum, average, and maximum values thereof. In certain embodiments, the red channel can have a spectral power distribution with the relative intensities of BP and BV increased or decreased within 30% greater or less, within 20% greater or less, within 10% greater or less, or within 5% greater or less than the values for one or more of Red Channels 1, 3-6, and 9-17 and the Exemplary Red Channels Average shown in Table 19 and Exemplary Red Channels A1-A50 and Exemplary Red Channels Averages A1 and A2 in Table 20A.

In some embodiments, the red channel can produce red light having a spectral power distribution with peak and valley intensities that fall between the Exemplary Red Channels Minimum and the Exemplary Red Channels Maximum shown in Table 19, In further embodiments, the red channel can produce red light having a spectral power distribution with relative ratios of intensity between particular pairs of the peak and valley intensities that fall between the Exemplary Red Channels Minimum and the Exemplary Red Channels Maximum values shown in Table 20. In certain embodiments, the red channel can have a spectral power distribution with the relative ratios of intensity between particular pairs of the peak and valley intensities increased or decreased within 30% greater or less, within 20% greater or less, within 10% greater or less, or within 5% greater or less than the relative ratio values for one or more of Red Channels 1, 3-6, and 9-17 and the Exemplary Red Channels Average shown in Table 20 and Exemplary Red Channels A1-A50 and Exemplary Red Channels Averages A1 and A2 in Table 20A. In some embodiments, the red channel can produce red light having a spectral power distribution with peak and valley intensities that fall between the Exemplary Red Channels Minimum A1 and the Exemplary Red Channels Maximum A1 shown in Table 20A.

In some embodiments, the red channel can produce red light having a spectral power distribution with peak and valley intensities that fall between the Exemplary Red Channels Minimum A2 and the Exemplary Red Channels Maximum A2 shown in Table 20A.

In one embodiment, the red channel has a blue peak at a wavelength between 420 nm and 465 nm, or between 445 nm and 460 nm, or at about 448 nm, or at about 449 nm; a blue valley at a wavelength between 470 nm and 505 nm, or at between 480 nm and 490 nm, or at about 481 nm, or at about 485 nm; and a red peak at a wavelength between 610 nm and 660 nm, or between 645 nm and 650 nm, or at about 649 nm, or at about 646 nm.

In one embodiment, the red channel has a relative spectral power distribution ratio for wavelengths (400<λ≤470)/(470<λ≤510) is between about 0,4 and about 15, or between about 1.2 and about 3.0, or between about 1.4 and about 3.0, or between about 2.7 and about 2.9, or between about 2.75 and about 2.80, or is about 1.5, or is about 2.78; a relative spectral power distribution ratio for wavelengths (530<λ≤570)/(470<λ≤510) is between about 12 and about 18, or between about 13 and about 16, or between about 15 and about 16, or between about 15.4 and about 15.5, or between about 13.0 and about 13.5, or is about 13.3, or is about 15.45; a relative spectral power distribution ratio for wavelengths (600<λ≤630)/(470<λ≤510) is between about 15 and about 100, or between about 40 and about 60, or between about 45 and about 55, or between about 48 and about 52, or between about 49 and about 51, or is about 46, or is about 50; and a relative spectral power distribution ratio for wavelengths (630<λ≤780)/(470<λ≤510) is between about 55 and about 300, or between about 100 and about 150, or between about 120 and about 140, or between about 130 and about 140, or between about 135 and about 138, or is about 137, or is about 128.

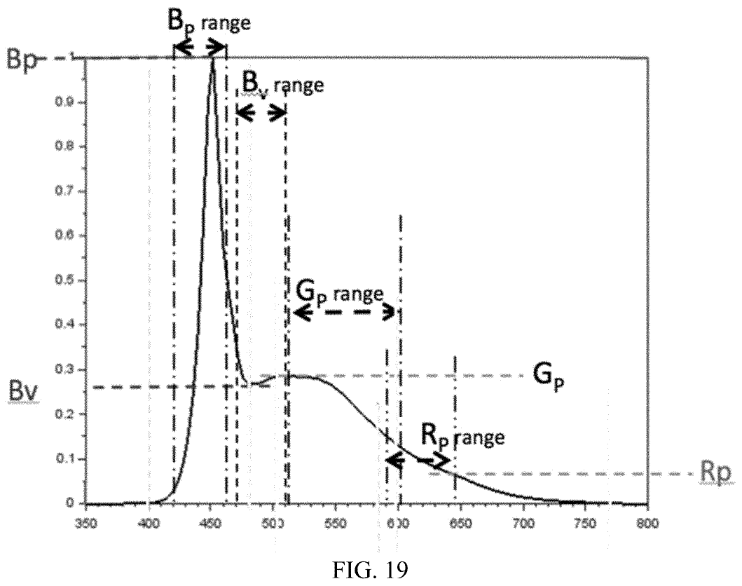

Tables 4A, and 44B and FIG. 19 show some aspects of exemplary blue lighting channels for some embodiments of the disclosure. In certain embodiments, a Blue Peak (BP) is present. In further embodiments, a Blue Valley (BV) is present. In some embodiments, a Red Peak (RP) is present. In some embodiments, a Green Peak (GP) is present. Table 21A shows the relative intensities of the peaks and valleys for exemplary blue lighting channels of the disclosure, with the BP values assigned an arbitrary value of 1.0 in the table. The wavelength at which each peak or valley is present is also shown in Table 21A. Table 21B shows the relative spectral power distributions within particular wavelength ranges, with values relative to the spectral power 470<λ≤510. In certain embodiments, the blue channel can have a spectral power distribution with the relative intensities of BP and BV increased or decreased within 30% greater or less, within 20% greater or less, within 10% greater or less, or within 5% greater or less than the values for one or more of Exemplary Blue Channels A1-A48 and the Exemplary Blue Channels Averages A1 and A2 shown in Table 21A. In some embodiments, the blue channel can produce blue light having a spectral power distribution with peak and valley intensities that fall between the Exemplary Blue Channels Minimum A1 and the Exemplary Blue Channels Maximum A1 shown in Table 21A. In some embodiments, the blue channel can produce blue light having a spectral power distribution with peak and valley intensities that fall between the Exemplary Blue Channels Minimum A2 and the Exemplary Blue Channels Maximum A2 shown in Table 21A. In certain embodiments, the blue channel can have a spectral power distribution with the relative ratios of intensity between particular pairs of the peak and valley intensities increased or decreased within 30% greater or less, within 20% greater or less, within 10% greater or less, or within 5% greater or less than the relative ratio values for one or more of Exemplary Blue Channels A1-A48 and the Exemplary Blue Channels Averages A1 and A2 shown in Table 21A. In some embodiments, the blue channel can produce blue light having a spectral power distribution with the relative spectral power distributions within particular wavelength ranges that fall between the Exemplary Blue Channels Minimum A1 and the Exemplary Blue Channels Maximum A1 shown in Table 21B. In some embodiments, the blue channel can produce blue light having a spectral power distribution with the relative spectral power distributions within particular wavelength ranges that fall between the Exemplary Blue Channels Minimum A2 and the Exemplary Blue Channels Maximum A2 shown in Table 21B.

In one embodiment, the blue channel has a blue peak at a wavelength between 420 nm and 480 nm, or between 420 nm and 465 nm, or between 445 nm and 460 nm, or at about 453 nm, or at about 457 nm; a blue valley at a wavelength between 470 nm and 515 nm, or between 490 nm and 510 nm, or at about 489 nm, or at about 503 nm; a green peak at a wavelength between 510 nm and 605 nm, or between 510 nm and 550 nm, or at about 511 nm, or at about 527 nm; and a red peak at a wavelength between 585 nm and 640 nm, or between 585 nm and 595 nm, or at about 591 nm.

In one embodiment, the blue channel has a relative spectral power distribution ratio for wavelengths (400<λ≤470)/(470<λ≤510) is between about 1.6 and about 60, or between about 1.6 and about 40, or between about 1.6 and about 20, or between about 1.6 and about 6, or between about 1.6 and about 2.1, or is about 2.0, or is about 1.7; a relative spectral power distribution ratio for wavelengths (530<λ≤570)/(470<λ≤510) is between about 0.37 and about 6.0, or between about 0.37 and about 4.0, or between about 0.75 and about 2.1, or between about 0.75 and about 0.80, or between about 0.72 and about 0.88, or is about 0.84, or is about 0.78; a relative spectral power distribution ratio for wavelengths (600<λ<630)/(470<λ≤510) is between about 0.25 and about 5.0, or between about 0.25 and about 11.5, or between about 0.25 and about 0.95, or between about 0.28 and about 0.30, or is between about 0.27 and about 0.31, or is about 0.30, or is about 0.28; and a relative spectral power distribution ratio for wavelengths (630<λ≤780)/(470<λ≤510) is between about 0.23 and about 1.5. or is between about 0.23 and about 4.0, or between about 0.24 and about 1.0, or between about 0.32 and about 0.33, or between about 0.32 and about 0.36, or is about 0.36, or is about 0.325.

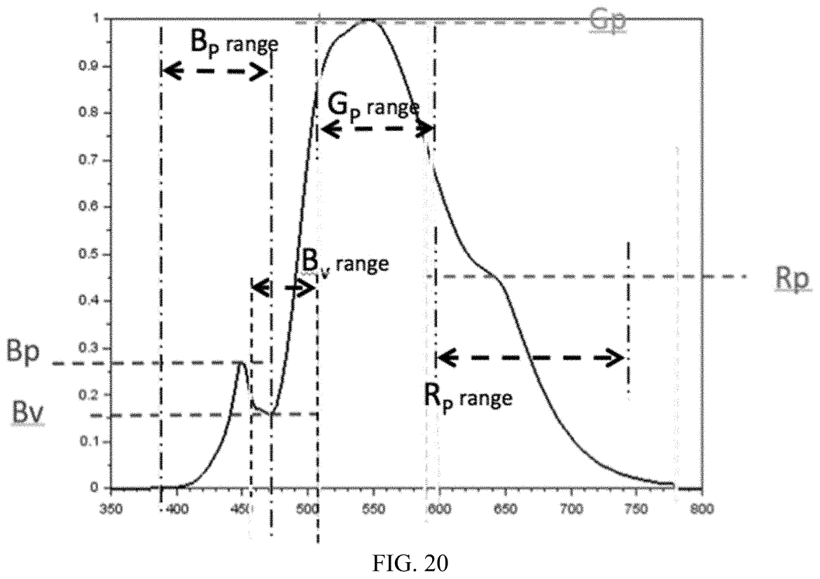

Tables 22A, and 22B and FIG. 20 show some aspects of exemplary short-blue-pumped cyan (also referred to as “SBC”) lighting channels for some embodiments of the disclosure. In certain embodiments, a Blue Peak (BP) is present. In further embodiments, a Blue Valley (By) is present. In some embodiments, a Red Peak (RP) is present. In some embodiments, a Green Peak (GP) is present. Table 22A shows the relative intensities of the peaks and valleys for exemplary SBC lighting channels of the disclosure, with the BP values assigned an arbitrary value of 1.0 in the table. The wavelength at which each peak or valley is present is also shown in Table 22A. Table 22B shows the relative spectral power distributions within particular wavelength ranges, with values relative to the spectral power 470<λ≤510. In certain embodiments, the SBC channel can have a spectral power distribution with the relative intensities of BP and BV increased or decreased within 30% greater or less, within 20% greater or less, within 10% greater or less, or within 5% greater or less than the values for one or more of Exemplary SBC Channels A1-A57 and the Exemplary SBC Channels Averages A1 and A2 shown in Table 22A. In some embodiments, the SBC channel can produce cyan light having a spectral power distribution with peak and valley intensities that fall between the Exemplary SBC Channels Minimum A1 and the Exemplary SBC Channels Maximum A1 shown in Table 22A. In some embodiments, the SBC channel can produce cyan light having a spectral power distribution with peak and valley intensities that fall between the Exemplary SBC Channels Minimum A2 and the Exemplary SBC Channels Maximum A2 shown in Table 22A. In certain embodiments, the SBC channel can have a spectral power distribution with the relative ratios of intensity between particular pairs of the peak and valley intensities increased or decreased within 30% greater or less, within 20% greater or less, within 10% greater or less, or within 5% greater or less than the relative ratio values for one or more of Exemplary SBC Channels A1-A57 and the Exemplary SBC Channels Averages A1 and A2 shown in Table 22A. In some embodiments, the SBC channel can produce cyan light having a spectral power distribution with the relative spectral power distributions within particular wavelength ranges that fall between the Exemplary SBC Channels Minimum A1 and the Exemplary SBC Channels Maximum A1 shown in Table 22B. In some embodiments, the SBC channel can produce cyan light having a spectral power distribution with the relative spectral power distributions within particular wavelength ranges that fall between the Exemplary SBC Channels Minimum A2 and the Exemplary SBC Channels Maximum A2 shown in Table 22B.

In one embodiment, the SBC channel has a blue peak at a wavelength between 420 nm and 465 nm, or between 445 nm and 465 nm, or at about 461 nm. or at about 453 nm; a blue valley at a wavelength between 470 nm and 500 nm, or between 470 nm and 480 nm or between 470 nm and 475 nm, or at about 471 nm, or at a wavelength between 515 nm and 605 nm; a green peak at a wavelength between 515 nm and 555 nm, or at about 553 nm, or at about 540 nm; and a red peak at a wavelength between 590 nm and 650 nm, or between 590 nm and 600 nm, or at about 591 nm.

In one embodiment, the SBC channel has a relative spectral power distribution ratio for wavelengths (400<λ≤470)/(470<λ≤510) is between about 0.1 and about 12, or between about 0.1 and about 1.0, or between about 0.2 and about 0.5, or between about 0.2 and about 0.3, or between about 0.25 and about 0.29, or is about 0.29, or is about 0.25; a relative spectral power distribution ratio for wavelengths (530<2 <570)/(470<λ≤510) is between about 1.5 and about 5.0, or between about 1.5 and about 3.0, or between about 1.8 and about 2.1, or between about 1.8 and about 1.9, or between about 2.0 and about 2.05, or is about 1.85, or is about 103; a relative spectral power distribution ratio for wavelengths (600<λ≤1630)/(470<λ≤510) is between about 0.4 and about 15, or between about 0.5 and about 2.0, or between about 0.7 and about 1.1, or between about 0.80 and about 0.86, or between about 0.75 and about 0.90, or is about 0.77, or is about 0.84; and a relative spectral power distribution ratio for wavelengths (630<λ≤780)/(470<λ≤510) is between about 0.1 and about 30, or between about 0.5 and about 2.0, or between about 0.8 and about 1.3, or between about 0,9 and about 1.1, or between about 0.95 and about 1.15, or is about 1.00, or is about 1.10.

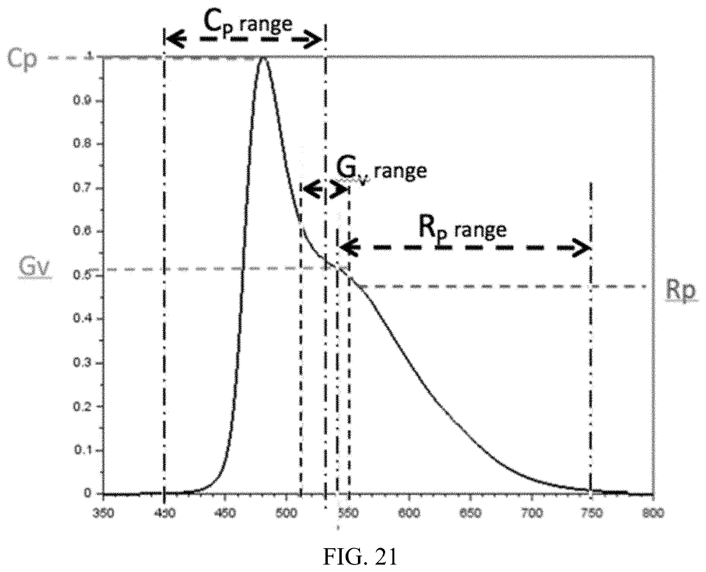

Tables 23A, and 23B and FIG. 21 show some aspects of exemplary long-blue-pumped cyan (also referred to as “LBC”) lighting channels for some embodiments of the disclosure. In certain embodiments, a Cyan Peak (CP) is present. In further embodiments, a Green Valley (GV) is present. In some embodiments, a Red Peak (RP) is present. Table 23A shows the relative intensities of the peaks and valleys for exemplary LBC lighting channels of the disclosure, with the CP values assigned an arbitrary value of 1.0 in the table. The wavelength at which each peak or valley is present is also shown in Table 23A. Table 23B shows the relative spectral power distributions within particular wavelength ranges, with values relative to the spectral power 470<λ≤510. In certain embodiments, the LBC channel can have a spectral power distribution with the relative intensities of CP and GV increased or decreased within 30% greater or less, within 20% greater or less, within 10% greater or less, or within 5% greater or less than the values for one or more of Exemplary LBC Channels A1-A58 and the Exemplary LBC Channels Averages A1 and A2 shown in Table 23A. In some embodiments, the LBC channel can produce cyan light having a spectral power distribution with peak and valley intensities that fall between the Exemplary LBC Channels Minimum A1 and the Exemplary LBC Channels Maximum A1 shown in Table 23A. In some embodiments, the LBC channel can produce cyan light having a spectral power distribution with peak and valley intensities that fall between the Exemplary LBC Channels Minimum A2 and the Exemplary LBC Channels Maximum A2 shown in Table 23A. In certain embodiments, the LBC channel can have a spectral power distribution with the relative ratios of intensity between particular pairs of the peak and valley intensities increased or decreased within 30% greater or less, within 20% greater or less, within 10% greater or less, or within 5% greater or less than the relative ratio values for one or more of Exemplary LBC Channels A1-A58 and the Exemplary LBC Channels Averages A1 and A2 shown in Table 23A. In some embodiments, the LBC channel can produce cyan light having a spectral power distribution with the relative spectral power distributions within particular wavelength ranges that fall between the Exemplary LBC Channels Minimum A1 and the Exemplary LBC Channels Maximum A1 shown in Table 23B. In some embodiments, the LBC channel can produce cyan light having a spectral power distribution with the relative spectral power distributions within particular wavelength ranges that fall between the Exemplary LBC Channels Minimum A2 and the Exemplary LBC Channels Maximum A2 shown in Table 23B.

In one embodiment, the LBC channel has a cyan peak at a wavelength between 470 nm and 520 nm, or between 475 nm and 485 nm, or at about 480 nm, or at 481 nm; a green valley at a wavelength between 530 nm and 600 nm, or between 580 nm and 600 nm, or at about 590 nm, or at 591 nm; and a red peak at a wavelength between 590 nm and 650 nm, or between 590 nm and 620 nm, or at about 590 nm, or at 591 nm

In one embodiment, the LBC channel has a relative spectral power distribution ratio for wavelengths (400<λ≤470)/(470<λ≤510) is between about 0,04 and about 0.4, or between about 0.20 and about 0.28, or between about 0.22 and about 0.25, or between about 0.22 and about 0.244, or between about 0.22 and about 0.225, or is about 0.22, or is about 0.24; a relative spectral power distribution ratio for wavelengths (530<λ≤570)/(470<λ≤510) is between about 0.13 and about 1.5, or between about 0.4 and about 0.8, or between about .55 and about 0.75, or between about 0.58 and about 0.70, or between about 0.68 and about 0.72, or is about 0.58, or is about 0.70; a relative spectral power distribution ratio for wavelengths (600<λ≤630)/(470<λ≤510) is between about 0.08 and about 0.8, or between about 0.20 and about 0.26 or between about 0.21 and about 0.24, or between about 0.235 and about 0.245, or between about 0.215 and about 0.220, or is about 0.217, or is about 0.241; and a relative spectral power distribution ratio for wavelengths (630<λ≤780)/(470<λ≤510) is between about 0,11 and about 1.4 or between about 0.20 and about 0,28, or between about 0.21 and about 0,23, or between about 0.25 and about 0.27, or between about 0.26 and about 0.265, or is about 0.23, or is about 0.26.

In some embodiments, the lighting devices of the disclosure can include a blue lighting channel, a red lighting channel, a short-blue-pumped lighting channel, and one or both of a saturated violet LED channel and a saturated cyan LED channel. The saturated violet LED channel can a peak wavelength of about 410 nm, or between about 380 nm and about 420 nm. The saturated cyan LED can have a peak wavelength of about 485 nm, or between about 460 nm and about 5 nm. In some embodiments, the saturated LED channels can have light emissions with FWHM of less than 40 nm, less than 35 nm, less than 30 nm, less than 25 nm, less than 20 nm, or less than 15 nm.

Some aspects of blue lighting channels suitable for use in these embodiments are shown in the Appendix of U.S. Provisional Application No. 62/885,162 as “Phosphor-Converted Blue”, “PCB”, or “Phosphor Blue” channels, with some aspects of spectral power distributions for some embodiments shown as graphical plots. Some aspects of red lighting channels suitable for use in these embodiments are shown in the Appendix as “Phosphor-Converted. Red”, “PCR”, or “Phosphor Red” channels, with some aspects of spectral power distributions for some embodiments shown as graphical plots. Some aspects of short-blue-pumped lighting channels suitable for use in these embodiments are shown in the Appendix as “Phosphor-Converted Green”, “PCG”, or “Phosphor Green” channels, with some aspects of spectral power distributions for some embodiments shown as graphical plots.

Those of ordinary skill in the art will appreciate that a variety of materials can be used in the manufacturing of the components in the devices and systems disclosed herein. Any suitable structure and/or material can be used for the various features described herein, and a skilled artisan will be able to select an appropriate structures and materials based on various considerations, including the intended use of the systems disclosed herein, the intended arena within which they will be used, and the equipment and/or accessories with which they are intended to be used, among other considerations. Conventional polymeric, metal-polymer composites, ceramics, and metal materials are suitable for use in the various components.

Materials hereinafter discovered and/or developed that are determined to be suitable for use in the features and elements described herein would also be considered acceptable.

When ranges are used herein for physical properties, such as molecular weight, or chemical properties, such as chemical formulae, all combinations, and subcombinations of ranges for specific exemplar therein are intended to be included.

The disclosures of each patent, patent application, and publication cited or described in this document are hereby incorporated herein by reference, in its entirety.

Those of ordinary skill in the art will appreciate that numerous changes and modifications can be made to the exemplars of the disclosure and that such changes and modifications can be made without departing from the spirit of the disclosure. It is, therefore, intended that the appended claims cover all such equivalent variations as fall within the true spirit and scope of the disclosure.

TABLE 1
Spectral Power Distribution for Wavelength Ranges (nm)

| | 380 < | 420 < | 460 < | 500 < | 540 < | 580 < | 620 < | 660 < | 700 < | 740 < |
|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ |
| | 420 | 460 | 500 | 540 | 580 | 620 | 660 | 700 | 740 | 780 |
| Blue minimum 1 | 0.3 | 100.0 | 0.8 | 15.2 | 25.3 | 26.3 | 15.1 | 5.9 | 1.7 | 0.5 |
| Blue maximum 1 | 110.4 | 100.0 | 196.1 | 61.3 | 59.2 | 70.0 | 80.2 | 22.1 | 10.2 | 4.1 |
| Red minimum 1 | 0.0 | 10.5 | 0.1 | 0.1 | 2.2 | 36.0 | 100.0 | 2.2 | 0.6 | 0.3 |
| Red maximum 1 | 2.0 | 1.4 | 3.1 | 7.3 | 22.3 | 59.8 | 100.0 | 61.2 | 18.1 | 5.2 |
| Short-blue-pumped cyan minimum 1 | 3.9 | 100.0 | 112.7 | 306.2 | 395.1 | 318.2 | 245.0 | 138.8 | 39.5 | 10.3 |
| Short-blue-pumped cyan maximum 1 | 130.6 | 100.0 | 553.9 | 2660.6 | 4361.9 | 3708.8 | 2223.8 | 712.2 | 285.6 | 99.6 |
| Short-blue-pumped cyan maximum 2 | 130.6 | 100.0 | 553.9 | 5472.8 | 9637.9 | 12476.9 | 13285.5 | 6324.7 | 1620.3 | 344.7 |
| Long-blue-pumped cyan minimum 1 | 0.0 | 0.0 | 100.0 | 76.6 | 38.0 | 33.4 | 19.6 | 7.1 | 2.0 | 0.6 |
| Long-blue-pumped cyan maximum 1 | 1.8 | 36.1 | 100.0 | 253.9 | 202.7 | 145.0 | 113.2 | 63.1 | 24.4 | 7.3 |

TABLE 2
Spectral Power Distribution for Wavelength Ranges (nm)

| | 380 < | 500 < | 600 < | 700 < |
|---|---|---|---|---|
| | λ ≤ | λ ≤ | λ ≤ | λ ≤ |
| | 500 | 600 | 700 | 780 |
| Blue minimum 1 | 100.0 | 27.0 | 19.3 | 20.5 |
| Blue maximum 1 | 100.0 | 74.3 | 46.4 | 51.3 |
| Red minimum 1 | 100.0 | 51.4 | 575.6 | 583.7 |
| Red maximum 1 | 100.0 | 2332.8 | 8482.2 | 9476.2 |
| Short-blue-pumped cyan minimum 1 | 100.0 | 279.0 | 170.8 | 192.8 |
| Short-blue-pumped cyan maximum 1 | 100.0 | 3567.4 | 4366.3 | 4696.6 |
| Long-blue-pumped cyan minimum 1 | 100.0 | 155.3 | 41.1 | 43.5 |
| Long-blue-pumped cyan maximum 1 | 100.0 | 503.0 | 213.2 | 243.9 |

TABLE 3
Spectral Power Distribution for Wavelength Ranges (nm)

| | 380 < | 400 < | 420 < | 440 < | 460 < | 480 < | 500 < | 520 < | 540 < | 560 < | 580 < |
|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ |
| Exemplary Color Channels | 400 | 420 | 440 | 460 | 480 | 500 | 520 | 540 | 560 | 580 | 600 |
| Blue Channel 1 | 0.1 | 1.2 | 20.6 | 100 | 49.2 | 35.7 | 37.2 | 36.7 | 33.4 | 26.5 | 19.8 |
| Red Channel 1 | 0.0 | 0.3 | 1.4 | 1.3 | 0.4 | 0.9 | 4.2 | 9.4 | 15.3 | 26.4 | 45.8 |
| Short-Blue-Pumped Cyan Channel 1 | 0.2 | 1.2 | 8.1 | 22.2 | 17.5 | 46.3 | 88.2 | 98.5 | 100.0 | 90.2 | 73.4 |
| Long-Blue-Pumped Cyan Channel 1 | 0.0 | 0.1 | 0.7 | 9.9 | 83.8 | 100 | 75.7 | 65.0 | 62.4 | 55.5 | 43.4 |
| Blue Channel 2 | 0.4 | 2.5 | 17.2 | 100 | 60.9 | 30.9 | 29.3 | 30.2 | 28.6 | 24.3 | 20.7 |
| Red Channel 2 | 0.1 | 0.4 | 1.1 | 3.4 | 3.6 | 2.7 | 5.9 | 11.0 | 16.9 | 28.1 | 46.8 |
| Short-Blue-Pumped Cyan Channel 2 | 0.5 | 0.6 | 3.4 | 13.5 | 16.6 | 47.2 | 83.7 | 95.8 | 100.0 | 95.8 | 86.0 |
| Long-Blue-Pumped Cyan Channel 2 | 0.1 | 0.2 | 1.0 | 9.1 | 54.6 | 100.0 | 99.6 | 75.7 | 65.5 | 56.8 | 48.9 |

| | 600 < | 620 < | 640 < | 660 < | 680 < | 700 < | 720 < | 740 < | 760 < | 780 < |
|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ |
| Exemplary Color Channels | 620 | 640 | 660 | 680 | 700 | 720 | 740 | 760 | 780 | 800 |
| Blue Channel 1 | 14.4 | 10.6 | 7.6 | 4.7 | 2.6 | 1.4 | 0.7 | 0.4 | 0.2 | 0.0 |
| Red Channel 1 | 66.0 | 87.0 | 100.0 | 72.5 | 42.0 | 22.3 | 11.6 | 6.1 | 3.1 | 0.0 |
| Short-Blue-Pumped Cyan Channel 1 | 57.0 | 48.1 | 41.4 | 27.0 | 15.1 | 7.9 | 4.0 | 2.1 | 1.0 | 0.0 |
| Long-Blue-Pumped Cyan Channel 1 | 30.9 | 21.5 | 14.5 | 8.5 | 4.5 | 2.4 | 1.3 | 0.7 | 0.3 | 0.0 |
| Blue Channel 2 | 18.5 | 16.6 | 13.6 | 9.5 | 6.0 | 3.5 | 2.0 | 1.2 | 0.8 | 0.0 |
| Red Channel 2 | 68.9 | 92.6 | 100.0 | 73.9 | 44.5 | 24.7 | 13.1 | 6.8 | 3.5 | 0.0 |
| Short-Blue-Pumped Cyan Channel 2 | 76.4 | 74.6 | 68.3 | 46.1 | 26.1 | 14.0 | 7.2 | 3.6 | 1.8 | 0.0 |
| Long-Blue-Pumped Cyan Channel 2 | 41.3 | 33.3 | 24.1 | 15.8 | 9.4 | 5.4 | 3.0 | 1.7 | 1.1 | 0.0 |

TABLE 4
Spectral Power Distribution for Wavelength Ranges (nm)

| | 380 < | 420 < | 460 < | 500 < | 540 < | 580 < | 620 < | 660 < | 700 < | 740 < |
|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ |
| Exemplary Color Channels | 420 | 460 | 500 | 540 | 580 | 620 | 660 | 700 | 740 | 780 |
| Red Channel 1 | 0.2 | 1.4 | 0.7 | 7.3 | 22.3 | 59.8 | 100.0 | 61.2 | 18.1 | 4.9 |
| Red Channel 2 | 1.8 | 4.2 | 2.7 | 7.2 | 19.3 | 59.1 | 100.0 | 59.5 | 20.4 | 5.9 |
| Blue Channel 1 | 1.1 | 100.0 | 70.4 | 61.3 | 49.7 | 28.4 | 15.1 | 6.0 | 1.7 | 0.5 |
| Blue Channel 2 | 25.7 | 100.0 | 69.4 | 31.6 | 38.7 | 38.3 | 33.7 | 14.9 | 5.6 | 2.0 |
| Short-Blue-Pumped Cyan Channel 1 | 0.7 | 15.9 | 33.5 | 98.2 | 100.0 | 68.6 | 47.1 | 22.1 | 6.3 | 1.7 |
| Short-Blue-Pumped Cyan Channel 2 | 30.3 | 100.0 | 313.2 | 1842.7 | 2770.2 | 2841.2 | 2472.2 | 1119.1 | 312.7 | 77.8 |
| Long-blue-pumped cyan Channel 1 | 0.0 | 5.8 | 100.0 | 76.6 | 64.1 | 40.4 | 19.6 | 7.1 | 2.0 | 0.6 |
| Long-blue-pumped cyan Channel 2 | 0.4 | 5.3 | 100.0 | 165.3 | 105.4 | 77.0 | 49.0 | 22.7 | 8.1 | 2.3 |

TABLE 5

| | | | LED | |
|---|---|---|---|---|
| | | | pump peak | |
| Exemplary Color Channels | ccx | ccy | wavelength | |
| Red Channel 1 | 0.5932 | 0.3903 | 450-455 | nm |
| Blue Channel 1 | 0.2333 | 0.2588 | 450-455 | nm |
| Long-Blue-Pumped Cyan Channel 1 | 0.2934 | 0.4381 | 505 | nm |
| Short-Blue-Pumped Cyan Channel 1 | 0.373 | 0.4978 | 450-455 | nm |
| Violet Channel 1 | 0.3585 | 0.3232 | 380 | nm |
| Violet Channel 2 | 0.3472 | 0.3000 | 400 | nm |
| Violet Channel 3 | 0.2933 | 0.2205 | 410 | nm |
| Violet Channel 4 | 0.3333 | 0.2868 | 420 | nm |
| Violet Channel 5 | | | 400 | nm |
| Yellow Channel 1 | 0.4191 | 0.5401 | 380 | nm |
| Yellow Channel 2 | 0.4218 | 0.5353 | 400 | nm |
| Yellow Channel 3 | 0.4267 | 0.5237 | 410 | nm |
| Yellow Channel 4 | 0.4706 | 0.4902 | 420 | nm |
| Yellow Channel 5 | | | 400 | nm |
| Yellow Channel 6 | | | 410 | nm |

TABLE 6

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| Red Channel 11 | 0.0 | 0.0 | 0.0 | 0.6 | 0.8 | 0.9 | 3.1 | 4.9 | 2.9 | 8.5 | 14.9 | 17.6 |
| Red Channel 3 | 0.0 | 0.0 | 0.0 | 0.0 | 0.1 | 3.9 | 14.9 | 3.4 | 0.5 | 0.8 | 2.0 | 5.8 |
| Red Channel 4 | 0.0 | 0.0 | 0.0 | 25.6 | 21.1 | 16.7 | 16.4 | 15.2 | 6.0 | 10.5 | 16.8 | 18.2 |
| Red Channel 5 | 0.0 | 0.0 | 0.0 | 0.7 | 1.0 | 12.6 | 68.4 | 23.0 | 5.5 | 16.7 | 35.7 | 43.0 |
| Red Channel 6 | 0.0 | 0.0 | 0.0 | 0.0 | 0.1 | 3.9 | 14.9 | 3.4 | 0.5 | 0.8 | 2.0 | 5.8 |
| Red Channel 7 | 0.0 | 0.0 | 0.0 | 2.0 | 15.5 | 13.4 | 2.8 | 0.9 | 1.0 | 3.2 | 5.7 | 7.8 |
| Red Channel 8 | 0.0 | 0.0 | 0.0 | 0.3 | 20.3 | 17.9 | 0.2 | 0.0 | 0.0 | 0.1 | 0.1 | 0.6 |
| Red Channel 9 | 0.0 | 0.0 | 0.0 | 0.0 | 0.0 | 0.4 | 4.1 | 5.8 | 4.0 | 7.2 | 12.7 | 18.9 |
| Red Channel 10 | 0.0 | 0.0 | 0.0 | 0.1 | 0.1 | 0.7 | 4.5 | 4.9 | 3.5 | 6.7 | 11.6 | 17.6 |
| Red Channel 1 | 0.0 | 0.0 | 0.0 | 0.0 | 0.3 | 1.4 | 1.3 | 0.4 | 0.9 | 4.2 | 9.4 | 15.3 |
| Red Channel 2 | 0.0 | 0.0 | 0.0 | 0.1 | 0.4 | 1.1 | 3.4 | 3.6 | 2.7 | 5.9 | 11.0 | 16.9 |
| Exemplary Red | 0.0 | 0.0 | 0.0 | 0.0 | 0.0 | 0.4 | 0.2 | 0.0 | 0.0 | 0.1 | 0.1 | 0.6 |
| Channels Minimum | | | | | | | | | | | | |
| Exemplary Red | 0.0 | 0.0 | 0.0 | 2.7 | 5.4 | 6.6 | 12.2 | 6.0 | 2.5 | 5.9 | 11.1 | 15.2 |
| Channels Average | | | | | | | | | | | | |
| Exemplary Red | 0.0 | 0.0 | 0.0 | 25.6 | 21.1 | 17.9 | 68.4 | 23.0 | 6.0 | 16.7 | 35.7 | 43.0 |
| Channels Maximum | | | | | | | | | | | | |
| | 560 < | 580 < | 600 < | 620 < | 640 < | 660 < | 680 < | 700 < | 720 < | 740 < | 760 < | 780 < |
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 580 | 600 | 620 | 640 | 660 | 680 | 700 | 720 | 740 | 760 | 780 | 800 |
| Red Channel 11 | 21.8 | 35.7 | 63.5 | 91.4 | 100.0 | 83.9 | 58.3 | 35.6 | 20.3 | 10.8 | 5.2 | 0.0 |
| Red Channel 3 | 11.8 | 30.2 | 64.2 | 94.6 | 100.0 | 83.6 | 58.7 | 36.3 | 21.0 | 11.4 | 6.0 | 0.0 |
| Red Channel 4 | 25.8 | 93.1 | 231.0 | 215.2 | 100.0 | 27.6 | 7.1 | 2.9 | 1.9 | 1.5 | 1.8 | 0.0 |
| Red Channel 5 | 47.5 | 100.0 | 478.3 | 852.3 | 100.0 | 12.4 | 4.5 | 2.7 | 1.9 | 1.5 | 1.0 | 0.0 |
| Red Channel 6 | 11.8 | 30.2 | 64.2 | 94.6 | 100.0 | 83.6 | 58.7 | 36.3 | 21.0 | 11.4 | 6.0 | 0.0 |
| Red Channel 7 | 13.0 | 28.9 | 59.4 | 89.8 | 100.0 | 84.5 | 58.8 | 36.0 | 20.5 | 10.9 | 5.2 | 0.0 |
| Red Channel 8 | 3.2 | 15.9 | 46.4 | 79.8 | 100.0 | 94.8 | 73.4 | 50.7 | 32.9 | 20.2 | 11.1 | 0.0 |
| Red Channel 9 | 29.4 | 46.9 | 72.4 | 95.7 | 100.0 | 83.0 | 57.2 | 34.7 | 19.7 | 10.8 | 5.7 | 0.0 |
| Red Channel 10 | 30.0 | 48.9 | 67.9 | 93.5 | 100.0 | 66.0 | 33.7 | 16.5 | 7.6 | 3.2 | 1.5 | 0.0 |
| Red Channel 1 | 26.4 | 45.8 | 66.0 | 87.0 | 100.0 | 72.5 | 42.0 | 22.3 | 11.6 | 6.1 | 3.1 | 0.0 |
| Red Channel 2 | 28.1 | 46.8 | 68.9 | 92.6 | 100.0 | 73.9 | 44.5 | 24.7 | 13.1 | 6.8 | 3.5 | 0.0 |
| Exemplary Red | 3.2 | 15.9 | 46.4 | 79.8 | 100.0 | 12.4 | 4.5 | 2.7 | 1.9 | 1.5 | 1.0 | 0.0 |
| Channels Minimum | | | | | | | | | | | | |
| Exemplary Red | 22.6 | 47.5 | 116.5 | 171.5 | 100.0 | 69.6 | 45.2 | 27.2 | 15.6 | 8.6 | 4.6 | 0.0 |
| Channels Average | | | | | | | | | | | | |
| Exemplary Red | 47.5 | 100.0 | 478.3 | 852.3 | 100.0 | 94.8 | 73.4 | 50.7 | 32.9 | 20.2 | 11.1 | 0.0 |
| Channels Maximum | | | | | | | | | | | | |

TABLE 7

| | 320 < | 380 < | 420 < | 460 < | 500 < | 540 < | 580 < | 620 < | 660 < | 700 < | 740 < |
|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 | 700 | 740 | 780 |
| Red Channel 11 | 0.0 | 0.7 | 2.1 | 4.1 | 12.2 | 20.5 | 51.8 | 100.0 | 74.3 | 29.3 | 8.4 |
| Red Channel 3 | 0.0 | 0.0 | 9.6 | 2.0 | 1.4 | 9.0 | 48.5 | 100.0 | 73.1 | 29.5 | 9.0 |
| Red Channel 4 | 0.0 | 14.8 | 10.5 | 6.7 | 8.7 | 14.0 | 102.8 | 100.0 | 11.0 | 1.5 | 1.1 |
| Red Channel 5 | 0.0 | 0.2 | 8.5 | 3.0 | 5.5 | 9.5 | 60.7 | 100.0 | 1.8 | 0.5 | 0.3 |
| Red Channel 6 | 0.0 | 0.0 | 9.6 | 2.0 | 1.4 | 9.0 | 48.5 | 100.0 | 73.1 | 29.5 | 9.0 |
| Red Channel 7 | 0.0 | 9.2 | 8.6 | 1.0 | 4.6 | 11.0 | 46.5 | 100.0 | 75.5 | 29.8 | 8.5 |
| Red Channel 8 | 0.0 | 11.5 | 10.1 | 0.1 | 0.1 | 2.1 | 34.6 | 100.0 | 93.6 | 46.5 | 17.5 |
| Red Channel 9 | 0.0 | 0.0 | 2.3 | 5.0 | 10.2 | 24.7 | 61.0 | 100.0 | 71.7 | 27.8 | 8.4 |
| Red Channel 10 | 0.0 | 0.1 | 2.7 | 4.3 | 9.5 | 24.6 | 60.4 | 100.0 | 51.5 | 12.4 | 2.4 |
| Red Channel 1 | 0.0 | 0.2 | 1.4 | 0.7 | 7.3 | 22.3 | 59.8 | 100.0 | 61.2 | 18.1 | 4.9 |
| Red Channel 2 | 0.0 | 0.3 | 2.3 | 3.3 | 8.8 | 23.4 | 60.1 | 100.0 | 61.5 | 19.6 | 5.3 |
| Exemplary Red | 0.0 | 0.0 | 1.4 | 0.1 | 0.1 | 2.1 | 34.6 | 100.0 | 1.8 | 0.5 | 0.3 |
| Channels Minimum | | | | | | | | | | | |
| Exemplary Red | 0.0 | 3.4 | 6.2 | 2.9 | 6.3 | 15.5 | 57.7 | 100.0 | 58.9 | 22.2 | 6.8 |
| Channels Average | | | | | | | | | | | |
| Exemplary Red | 0.0 | 14.8 | 10.5 | 6.7 | 12.2 | 24.7 | 102.8 | 100.0 | 93.6 | 46.5 | 17.5 |
| Channels Maximum | | | | | | | | | | | |

TABLE 8

| | 320 < | 400 < | 500 < | 600 < | 700 < |
|---|---|---|---|---|---|
| | λ ≤ | λ ≤ | λ ≤ | λ ≤ | λ ≤ |
| | 400 | 500 | 600 | 700 | 780 |
| Red Channel 11 | 0.2 | 3.2 | 24.8 | 100.0 | 18.1 |
| Red Channel 3 | 0.0 | 5.7 | 12.6 | 100.0 | 18.7 |
| Red Channel 4 | 4.4 | 13.0 | 28.3 | 100.0 | 1.4 |
| Red Channel 5 | 0.1 | 7.6 | 16.8 | 100.0 | 0.5 |
| Red Channel 6 | 0.0 | 5.7 | 12.6 | 100.0 | 18.7 |
| Red Channel 7 | 0.5 | 8.6 | 14.9 | 100.0 | 18.5 |
| Red Channel 8 | 0.1 | 9.8 | 5.1 | 100.0 | 29.2 |
| Red Channel 9 | 0.0 | 3.5 | 28.2 | 100.0 | 17.3 |
| Red Channel 10 | 0.0 | 3.8 | 31.8 | 100.0 | 8.0 |
| Red Channel 1 | 0.0 | 1.2 | 27.5 | 100.0 | 11.7 |
| Red Channel 2 | 0.0 | 2.9 | 28.6 | 100.0 | 12.7 |
| Exemplary Red | 0.0 | 1.2 | 5.1 | 100.0 | 0.5 |
| Channels Minimum | | | | | |
| Exemplary Red | 0.5 | 6.2 | 20.3 | 100.0 | 14.2 |
| Channels Average | | | | | |
| Exemplary Red | 4.4 | 13.0 | 31.8 | 100.0 | 29.2 |
| Channels Maximum | | | | | |

TABLE 9

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| Violet Channel 1 | 0.0 | 51.7 | 633.8 | 545.9 | 100.0 | 53.3 | 53.9 | 10.5 | 6.9 | 22.4 | 40.4 | 48.0 |
| Violet Channel 2 | 0.0 | 0.3 | 11.0 | 116.1 | 100.0 | 17.8 | 2.7 | 0.5 | 1.1 | 4.4 | 7.9 | 9.4 |
| Violet Channel 5 | 0.0 | 0.3 | 10.9 | 115.7 | 100.0 | 23.4 | 10.2 | 1.9 | 1.4 | 4.5 | 8.2 | 9.7 |
| Violet Channel 3 | 0.0 | 0.0 | 1.4 | 29.4 | 100.0 | 29.8 | 4.6 | 0.8 | 0.9 | 3.3 | 6.0 | 7.0 |
| Violet Channel 4 | 0.0 | 1.0 | 1.9 | 10.7 | 100.0 | 86.0 | 15.7 | 2.7 | 3.7 | 13.8 | 24.8 | 28.4 |
| Exemplary Violet | 0.0 | 0.0 | 1.4 | 10.7 | 100.0 | 17.8 | 2.7 | 0.5 | 0.9 | 3.3 | 6.0 | 7.0 |
| Channels Minimum | | | | | | | | | | | | |
| Exemplary Violet | 0.0 | 10.7 | 131.8 | 163.6 | 100.0 | 42.1 | 17.4 | 3.3 | 2.8 | 9.7 | 17.4 | 20.5 |
| Channels Average | | | | | | | | | | | | |
| Exemplary Violet | 0.0 | 51.7 | 633.8 | 545.9 | 100.0 | 86.0 | 53.9 | 10.5 | 6.9 | 22.4 | 40.4 | 48.0 |
| Channels Maximum | | | | | | | | | | | | |
| | 560 < | 580 < | 600 < | 620 < | 640 < | 660 < | 680 < | 700 < | 720 < | 740 < | 760 < | 780 < |
| | λ ≤ | λ ≤ | λ ≤ | λ ≤ | λ ≤ | λ ≤ | λ ≤ | λ ≤ | λ ≤ | λ ≤ | λ ≤ | λ ≤ |
| Violet Channel 1 | 580 | 600 | 620 | 640 | 660 | 680 | 700 | 720 | 740 | 760 | 780 | 800 |
| Violet Channel 2 | 51.7 | 54.0 | 51.2 | 41.8 | 29.8 | 19.4 | 11.6 | 6.8 | 3.7 | 2.0 | 1.1 | 0.0 |
| Violet Channel 5 | 10.0 | 10.4 | 9.8 | 8.0 | 5.7 | 3.7 | 2.2 | 1.3 | 0.7 | 0.4 | 0.2 | 0.0 |
| Violet Channel 3 | 10.6 | 11.2 | 10.8 | 8.9 | 6.3 | 4.1 | 2.5 | 1.4 | 0.8 | 0.4 | 0.2 | 0.0 |
| Violet Channel 4 | 7.3 | 7.3 | 6.7 | 5.4 | 3.8 | 2.5 | 1.5 | 0.9 | 0.5 | 0.3 | 0.1 | 0.0 |
| Exemplary Violet | 28.0 | 29.9 | 32.6 | 20.3 | 10.7 | 6.5 | 3.9 | 2.4 | 1.4 | 0.8 | 0.5 | 0.0 |
| Channels Minimum | | | | | | | | | | | | |
| Exemplary Violet | 7.3 | 7.3 | 6.7 | 5.4 | 3.8 | 2.5 | 1.5 | 0.9 | 0.5 | 0.3 | 0.1 | 0.0 |
| Channels Average | | | | | | | | | | | | |
| Exemplary Violet | 21.5 | 22.6 | 22.2 | 16.9 | 11.3 | 7.2 | 4.3 | 2.6 | 1.4 | 0.8 | 0.5 | 0.0 |
| Channels Maximum | | | | | | | | | | | | |

TABLE 10

| | 320 < | 380 < | 420 < | 460 < | 500 < | 540 < | 580 < | 620 < | 660 < | 700 < | 740 < |
|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 | 700 | 740 | 780 |
| Violet Channel 1 | 106.1 | 100.0 | 16.6 | 2.7 | 9.7 | 15.4 | 16.3 | 11.1 | 4.8 | 1.6 | 0.5 |
| Violet Channel 2 | 5.2 | 100.0 | 9.5 | 0.8 | 5.7 | 9.0 | 9.3 | 6.3 | 2.7 | 0.9 | 0.3 |
| Violet Channel 5 | 5.2 | 100.0 | 15.6 | 1.5 | 5.9 | 9.4 | 10.2 | 7.1 | 3.1 | 1.0 | 0.3 |
| Violet Channel 3 | 1.1 | 100.0 | 26.6 | 1.3 | 7.1 | 11.0 | 10.8 | 7.1 | 3.0 | 1.0 | 0.3 |
| Violet Channel 4 | 2.6 | 100.0 | 91.9 | 5.8 | 34.8 | 50.9 | 56.4 | 28.0 | 9.4 | 3.4 | 1.2 |
| Exemplary Violet | 1.1 | 100.0 | 9.5 | 0.8 | 5.7 | 9.0 | 9.3 | 6.3 | 2.7 | 0.9 | 0.3 |
| Channels Minimum | | | | | | | | | | | |
| Exemplary Violet | 24.1 | 100.0 | 32.0 | 2.4 | 12.6 | 19.2 | 20.6 | 11.9 | 4.6 | 1.6 | 0.5 |
| Channels Average | | | | | | | | | | | |
| Exemplary Violet | 106.1 | 100.0 | 91.9 | 5.8 | 34.8 | 50.9 | 56.4 | 28.0 | 9.4 | 3.4 | 1.2 |
| Channels Maximum | | | | | | | | | | | |

TABLE 11

| | 320 < | 400 < | 500 < | 600 < | 700 < |
|---|---|---|---|---|---|
| | λ ≤ | λ ≤ | λ ≤ | λ ≤ | λ ≤ |
| | 400 | 500 | 600 | 700 | 780 |
| Violet Channel 1 | 548.2 | 100.0 | 96.4 | 68.5 | 6.1 |
| Violet Channel 2 | 104.3 | 100.0 | 34.4 | 24.0 | 2.1 |
| Violet Channel 5 | 92.7 | 100.0 | 32.3 | 23.8 | 2.1 |
| Violet Channel 3 | 22.7 | 100.0 | 22.7 | 14.5 | 1.3 |
| Violet Channel 4 | 6.5 | 100.0 | 59.9 | 35.6 | 2.5 |
| Exemplary Violet | 6.5 | 100.0 | 22.7 | 14.5 | 1.3 |
| Channels Minimum | | | | | |
| Exemplary Violet | 154.9 | 100.0 | 49.2 | 33.3 | 2.8 |
| Channels Average | | | | | |
| Exemplary Violet | 548.2 | 100.0 | 96.4 | 68.5 | 6.1 |
| Channels Maximum | | | | | |

TABLE 12

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| Yellow Channel 1 | 0.0 | 2.0 | 24.3 | 20.9 | 3.9 | 2.6 | 2.8 | 1.3 | 14.6 | 55.3 | 92.6 | 100.0 |
| Yellow Channel 2 | 0.0 | 0.1 | 2.3 | 24.3 | 20.9 | 3.7 | 0.6 | 1.8 | 17.7 | 55.3 | 89.8 | 100.0 |
| Yellow Channel 5 | 0.0 | 0.1 | 2.2 | 23.4 | 20.3 | 5.4 | 3.0 | 0.9 | 11.3 | 48.1 | 87.3 | 100.0 |
| Yellow Channel 3 | 0.0 | 0.0 | 0.4 | 9.2 | 31.4 | 9.4 | 1.4 | 0.6 | 11.3 | 48.2 | 87.5 | 100.0 |
| Yellow Channel 6 | 0.0 | 0.1 | 0.6 | 9.6 | 32.4 | 9.7 | 1.6 | 0.7 | 11.3 | 47.9 | 87.1 | 100.0 |
| Yellow Channel 4 | 0.0 | 5.0 | 8.0 | 7.1 | 9.4 | 7.6 | 3.6 | 2.2 | 11.8 | 48.2 | 87.2 | 100.0 |
| Exemplary Yellow | 0.0 | 0.0 | 0.4 | 7.1 | 3.9 | 2.6 | 0.6 | 0.6 | 11.3 | 47.9 | 87.1 | 100.0 |
| Channels Minimum | | | | | | | | | | | | |
| Exemplary Yellow | 0.0 | 1.2 | 6.3 | 15.8 | 19.7 | 6.4 | 2.2 | 1.3 | 13.0 | 50.5 | 88.6 | 100.0 |
| Channels Average | | | | | | | | | | | | |
| Exemplary Yellow | 0.0 | 5.0 | 24.3 | 24.3 | 32.4 | 9.7 | 3.6 | 2.2 | 17.7 | 55.3 | 92.6 | 100.0 |
| Channels Maximum | | | | | | | | | | | | |
| | 560 < | 580 < | 600 < | 620 < | 640 < | 660 < | 680 < | 700 < | 720 < | 740 < | 760 < | 780 < |
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 580 | 600 | 620 | 640 | 660 | 680 | 700 | 720 | 740 | 760 | 780 | 800 |
| Yellow Channel 1 | 91.4 | 77.7 | 61.5 | 44.6 | 30.0 | 19.6 | 11.8 | 7.3 | 4.1 | 2.3 | 1.3 | 0.0 |
| Yellow Channel 2 | 94.2 | 80.8 | 63.6 | 45.9 | 30.7 | 20.0 | 12.1 | 7.5 | 4.2 | 2.4 | 1.5 | 0.0 |
| Yellow Channel 5 | 96.7 | 85.5 | 69.3 | 51.0 | 34.5 | 22.6 | 13.7 | 8.4 | 4.7 | 2.7 | 1.5 | 0.0 |
| Yellow Channel 3 | 95.8 | 83.2 | 66.2 | 47.9 | 32.2 | 21.0 | 12.8 | 7.9 | 4.5 | 2.6 | 1.5 | 0.0 |
| Yellow Channel 6 | 97.4 | 88.6 | 77.3 | 64.1 | 49.6 | 35.4 | 22.7 | 14.0 | 7.9 | 4.4 | 2.4 | 0.0 |
| Yellow Channel 4 | 99.9 | 113.9 | 134.0 | 80.5 | 39.5 | 23.2 | 13.9 | 8.6 | 5.0 | 3.0 | 2.0 | 0.0 |
| Exemplary Yellow | 91.4 | 77.7 | 61.5 | 44.6 | 30.0 | 19.6 | 11.8 | 7.3 | 4.1 | 2.3 | 1.3 | 0.0 |
| Channels Minimum | | | | | | | | | | | | |
| Exemplary Yellow | 95.9 | 88.3 | 78.7 | 55.7 | 36.1 | 23.6 | 14.5 | 9.0 | 5.1 | 2.9 | 1.7 | 0.0 |
| Channels Average | | | | | | | | | | | | |
| Exemplary Yellow | 99.9 | 113.9 | 134.0 | 80.5 | 49.6 | 35.4 | 22.7 | 14.0 | 7.9 | 4.4 | 2.4 | 0.0 |
| Channels Maximum | | | | | | | | | | | | |

TABLE 13

| | 320 < | 380 < | 420 < | 460 < | 500 < | 540 < | 580 < | 620 < | 660 < | 700 < | 740 < |
|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 | 700 | 740 | 780 |
| Yellow Channel 1 | 13.7 | 12.9 | 2.8 | 8.3 | 77.2 | 100.0 | 72.7 | 39.0 | 16.4 | 5.9 | 1.9 |
| Yellow Channel 2 | 1.2 | 23.3 | 2.2 | 10.1 | 74.7 | 100.0 | 74.4 | 39.5 | 16.5 | 6.0 | 2.0 |
| Yellow Channel 5 | 1.2 | 22.2 | 4.3 | 6.2 | 68.8 | 100.0 | 78.7 | 43.5 | 18.4 | 6.7 | 2.2 |
| Yellow Channel 3 | 0.2 | 20.8 | 5.5 | 6.1 | 69.3 | 100.0 | 76.3 | 40.9 | 17.3 | 6.3 | 2.1 |
| Yellow Channel 6 | 0.3 | 21.3 | 5.7 | 6.0 | 68.4 | 100.0 | 84.1 | 57.6 | 29.5 | 11.1 | 3.4 |
| Yellow Channel 4 | 6.5 | 8.3 | 5.6 | 7.0 | 67.7 | 100.0 | 124.1 | 60.1 | 18.6 | 6.8 | 2.5 |
| Exemplary Yellow | 0.2 | 8.3 | 2.2 | 6.0 | 67.7 | 100.0 | 72.7 | 39.0 | 16.4 | 5.9 | 1.9 |
| Channels Minimum | | | | | | | | | | | |
| Exemplary Yellow | 3.9 | 18.1 | 4.4 | 7.3 | 71.0 | 100.0 | 85.0 | 46.7 | 19.4 | 7.1 | 2.3 |
| Channels Average | | | | | | | | | | | |
| Exemplary Yellow | 13.7 | 23.3 | 5.7 | 10.1 | 77.2 | 100.0 | 124.1 | 60.1 | 29.5 | 11.1 | 3.4 |
| Channels Maximum | | | | | | | | | | | |

TABLE 14

| | 320 < | 400 < | 500 < | 600 < | 700 < |
|---|---|---|---|---|---|
| | λ ≤ | λ ≤ | λ ≤ | λ ≤ | λ ≤ |
| | 400 | 500 | 600 | 700 | 780 |
| Yellow Channel 1 | 11.3 | 6.1 | 100.0 | 40.2 | 3.6 |
| Yellow Channel 2 | 6.3 | 10.7 | 100.0 | 41.0 | 3.7 |
| Yellow Channel 5 | 6.2 | 9.8 | 100.0 | 45.8 | 4.2 |
| Yellow Channel 3 | 2.3 | 13.0 | 100.0 | 43.4 | 4.0 |
| Yellow Channel 6 | 2.4 | 13.2 | 100.0 | 59.2 | 6.8 |
| Yellow Channel 4 | 4.5 | 7.7 | 100.0 | 64.8 | 4.1 |
| Exemplary Yellow | 2.3 | 6.1 | 100.0 | 40.2 | 3.6 |
| Channels Minimum | | | | | |
| Exemplary Yellow | 5.5 | 10.1 | 100.0 | 49.1 | 4.4 |
| Channels Average | | | | | |
| Exemplary Yellow | 11.3 | 13.2 | 100.0 | 64.8 | 6.8 |
| Channels Maximum | | | | | |

TABLE 15

| | Violet Peak (Vp) | | Violet Valley (Vv) | | Green Peak (Gp) | | Red Valley (Rv) | |
|---|---|---|---|---|---|---|---|---|
| | 380 < λ ≤ 460 | | 450 < λ ≤ 510 | | 500 < λ ≤ 650 | | 650 < λ ≤ 780 | |
| | λ | Vp | λ | Vv | λ | Gp | λ | Rv |
| Violet Channel 1 | 380 | 1 | 486 | 0.00485 | 596 | 0.05521 | 751 | 0.00218 |
| Violet Channel 2 | 400 | 1 | 476 | 0.00185 | 592 | 0.05795 | 751 | 0.00227 |
| Violet Channel 5 | 400 | 1 | 482 | 0.00525 | 596 | 0.06319 | 751 | 0.00252 |
| Violet Channel 3 | 410 | 1 | 477 | 0.00368 | 578 | 0.06123 | 751 | 0.00232 |
| Violet Channel 4 | 420 | 1 | 477 | 0.01032 | 608 | 0.22266 | 749 | 0.00519 |
| Exemplary Violet | 380 | 1 | 476 | 0.00185 | 578 | 0.05521 | 749 | 0.00218 |
| Channels Minimum | | | | | | | | |
| Exemplary Violet | 402 | 1 | 480 | 0.00519 | 594 | 0.09205 | 751 | 0.00290 |
| Channels Average | | | | | | | | |
| Exemplary Violet | 420 | 1 | 486 | 0.01032 | 608 | 0.22266 | 751 | 0.00519 |
| Channels Maximum | | | | | | | | |

TABLE 16

| | Ratio | | | | |
|---|---|---|---|---|---|
| | Vp/Vv | Vp/Gp | Vp/Rv | Gp/Vv | Gp/Rv |
| Violet Channel 1 | 206.3 | 18.1 | 458.5 | 11.4 | 25.3 |
| Violet Channel 2 | 540.0 | 17.3 | 440.3 | 31.3 | 25.5 |
| Violet Channel 5 | 190.4 | 15.8 | 397.0 | 12.0 | 25.1 |
| Violet Channel 3 | 272.0 | 16.3 | 431.8 | 16.7 | 26.4 |
| Violet Channel 4 | 96.9 | 4.5 | 192.6 | 21.6 | 42.9 |
| Exemplary Violet | 96.9 | 4.5 | 192.6 | 11.4 | 25.1 |
| Channels Minimum | | | | | |
| Exemplary Violet | 261.1 | 14.4 | 384.0 | 18.6 | 29.0 |
| Channels Average | | | | | |
| Exemplary Violet | 540.0 | 18.1 | 458.5 | 31.3 | 42.9 |
| Channels Maximum | | | | | |

TABLE 17

| | Violet Peak | | Violet Valley | | Green Peak | |
|---|---|---|---|---|---|---|
| | 330 < λ ≤ 430 | | 420 < λ ≤ 510 | | 500 < λ ≤ 780 | |
| | λ | Vp | λ | Vv | λ | Gp |
| Yellow Channel 1 | 380 | 0.37195 | 470 | 0.00534 | 548 | 1 |
| Yellow Channel 2 | 400 | 0.37612 | 458 | 0.00275 | 549 | 1 |
| Yellow Channel 5 | 400 | 0.36297 | 476 | 0.00317 | 561 | 1 |
| Yellow Channel 3 | 410 | 0.37839 | 476 | 0.00139 | 547 | 1 |
| Yellow Channel 6 | 410 | 0.38876 | 476 | 0.00223 | 561 | 1 |
| Yellow Channel 4 | 419 | 0.07831 | 476 | 0.01036 | 608 | 1 |
| Exemplary Yellow | 380 | 0.07831 | 458 | 0.00139 | 547 | 1 |
| Channels Minimum | | | | | | |
| Exemplary Yellow | 403 | 0.32608 | 472 | 0.00421 | 562 | 1 |
| Channels Average | | | | | | |
| Exemplary Yellow | 419 | 0.38876 | 476 | 0.01036 | 608 | 1 |
| Channels Maximum | | | | | | |

TABLE 18

| | Ratio | | |
|---|---|---|---|
| | Vp/Vv | Vp/Gp | Gp/Vv |
| Yellow Channel 1 | 69.7 | 0.372 | 187.3 |
| Yellow Channel 2 | 136.9 | 0.376 | 364.0 |
| Yellow Channel 5 | 114.4 | 0.363 | 315.3 |
| Yellow Channel 3 | 273.2 | 0.378 | 722.0 |
| Yellow Channel 6 | 174.3 | 0.389 | 448.2 |
| Yellow Channel 4 | 7.6 | 0.078 | 96.5 |
| Exemplary Yellow | 7.559 | 0.078 | 96.525 |
| Channels Minimum | | | |
| Exemplary Yellow | 129.336 | 0.326 | 355.556 |
| Channels Average | | | |
| Exemplary Yellow | 273.202 | 0.389 | 722.022 |
| Channels Maximum | | | |

TABLE 19

| | Blue Peak | | Blue Valley | | Red Peak | |
|---|---|---|---|---|---|---|
| | 380 < λ ≤ 460 | | 450 < λ ≤ 510 | | 500 < λ ≤ 780 | |
| | λ | Bp | λ | Bv | λ | Rp |
| Red Channel 11 | 461 | 0.05898 | 488 | 0.02327 | 649 | 1 |
| Red Channel 3 | 449 | 0.18404 | 497 | 0.00309 | 640 | 1 |
| Red Channel 4 | 461 | 0.07759 | 495 | 0.01753 | 618 | 1 |
| Red Channel 5 | 453 | 0.07508 | 494 | 0.00374 | 628 | 1 |
| Red Channel 6 | 449 | 0.18404 | 497 | 0.00309 | 640 | 1 |
| Red Channel 9 | 461 | 0.07737 | 489 | 0.03589 | 645 | 1 |
| Red Channel 10 | 461 | 0.06982 | 489 | 0.02971 | 645 | 1 |
| Red Channel 1 | 445 | 0.01599 | 477 | 0.00353 | 649 | 1 |
| Red Channel 12 | 445 | 0.01217 | 477 | 0.00203 | 649 | 1 |
| Red Channel 13 | 451 | 0.06050 | 479 | 0.01130 | 651 | 1 |
| Red Channel 14 | 449 | 0.06020 | 485 | 0.00612 | 653 | 1 |
| Red Channel 15 | 445 | 0.02174 | 477 | 0.00326 | 649 | 1 |
| Red Channel 16 | 450 | 0.03756 | 483 | 0.00388 | 643 | 1 |
| Red Channel 17 | 450 | 0.03508 | 485 | 0.00425 | 641 | 1 |
| Exemplary Red | 445 | 0.01217 | 477 | 0.00203 | 618 | 1 |
| Channels Minimum | | | | | | |
| Exemplary Red | 452 | 0.06930 | 487 | 0.01076 | 643 | 1 |
| Channels Average | | | | | | |
| Exemplary Red | 461 | 0.18404 | 497 | 0.03589 | 653 | 1 |
| Channels Maximum | | | | | | |

TABLE 20

| | Ratios | | |
|---|---|---|---|
| | Bp/Bv | Bp/Rp | Rp/Bv |
| Red Channel 11 | 2.5 | 0.059 | 43.0 |
| Red Channel 3 | 59.5 | 0.184 | 323.3 |
| Red Channel 4 | 4.4 | 0.078 | 57.1 |
| Red Channel 5 | 20.1 | 0.075 | 267.7 |
| Red Channel 6 | 59.5 | 0.184 | 323.3 |
| Red Channel 9 | 2.2 | 0.077 | 27.9 |
| Red Channel 10 | 2.4 | 0.070 | 33.7 |
| Red Channel 1 | 4.5 | 0.016 | 283.3 |
| Red Channel 12 | 6.0 | 0.012 | 493.0 |
| Red Channel 13 | 5.4 | 0.061 | 88.5 |
| Red Channel 14 | 9.8 | 0.060 | 163.4 |
| Red Channel 15 | 6.7 | 0.022 | 306.3 |
| Red Channel 16 | 9.7 | 0.038 | 257.7 |
| Red Channel 17 | 8.3 | 0.035 | 235.5 |
| Exemplary Red | 2.156 | 0.012 | 27.864 |
| Channels Minimum | | | |
| Exemplary Red | 14.349 | 0.069 | 207.398 |
| Channels Average | | | |
| Exemplary Red | 59.501 | 0.184 | 492.975 |
| Channels Maximum | | | |

TABLE 20A

| | Bp | | Bv | | Rp | |
|---|---|---|---|---|---|---|
| | Bp | wavelength | Bv | wavelength | Rp | wavelength |
| Exemplary Red Channel A1 | 0.12305 | 461 | 0.021887 | 496 | 1 | 621 |
| Exemplary Red Channel A2 | 0.068064 | 446 | 0.011939 | 487 | 1 | 622 |
| Exemplary Red Channel A3 | 0.113313 | 417 | 0.011355 | 487 | 1 | 610 |
| Exemplary Red Channel A4 | 0.075081 | 453 | 0.003735 | 494 | 1 | 628 |
| Exemplary Red Channel A5 | 0.185194 | 449 | 0.009764 | 488 | 1 | 615 |
| Exemplary Red Channel A6 | 0.192537 | 461 | 0.033133 | 496 | 1 | 614 |
| Exemplary Red Channel A7 | 0.184038 | 449 | 0.003093 | 497 | 1 | 640 |
| Exemplary Red Channel A8 | 0.195877 | 420 | 0.013254 | 471 | 1 | 649 |
| Exemplary Red Channel A9 | 0.234381 | 420 | 0.007149 | 479 | 1 | 649 |
| Exemplary Red Channel A10 | 0.058976 | 461 | 0.023271 | 488 | 1 | 649 |
| Exemplary Red Channel A11 | 0.058976 | 461 | 0.023271 | 488 | 1 | 649 |
| Exemplary Red Channel A12 | 0.143509 | 446 | 0.009247 | 479 | 1 | 616 |
| Exemplary Red Channel A13 | 0.373357158 | 421 | 0.00335497 | 483 | 1 | 654 |
| Exemplary Red Channel A14 | 0.184038 | 449 | 0.003093 | 497 | 1 | 640 |
| Exemplary Red Channel A15 | 0.058976 | 461 | 0.023271 | 488 | 1 | 649 |
| Exemplary Red Channel A16 | 0.184038 | 449 | 0.003093 | 497 | 1 | 640 |
| Exemplary Red Channel A17 | 0.058976 | 461 | 0.023271 | 488 | 1 | 649 |
| Exemplary Red Channel A18 | 0.058976 | 461 | 0.023271 | 488 | 1 | 649 |
| Exemplary Red Channel A19 | 0.0773731 | 461 | 0.0358889 | 489 | 1 | 645 |
| Exemplary Red Channel A20 | 0.069817 | 461 | 0.0297083 | 489 | 1 | 645 |
| Exemplary Red Channel A21 | 0.184038 | 449 | 0.003093 | 497 | 1 | 640 |
| Exemplary Red Channel A22 | 0.0605 | 451 | 0.0113 | 479 | 1 | 652 |
| Exemplary Red Channel A23 | 0.0602045 | 449 | 0.006121351 | 485 | 1 | 653 |
| Exemplary Red Channel A24 | 0.0217433 | 445 | 0.0032648 | 477 | 1 | 649 |
| Exemplary Red Channel A25 | 0.0159937 | 445 | 0.0035293 | 477 | 1 | 649 |
| Exemplary Red Channel A26 | 0.035076973 | 450 | 0.004246805 | 485 | 1 | 641 |
| Exemplary Red Channel A27 | 0.037557029 | 450 | 0.003880344 | 483 | 1 | 643 |
| Exemplary Red Channel A28 | 0.0159937 | 445 | 0.0035293 | 477 | 1 | 649 |
| Exemplary Red Channel A29 | 0.0146951 | 441 | 0.0053371 | 477 | 1 | 649 |
| Exemplary Red Channel A30 | 0.029 | 449 | 0.0053 | 485 | 1 | 641 |
| Exemplary Red Channel A31 | 0.0189 | 445 | 0.0032 | 483 | 1 | 645 |
| Exemplary Red Channel A32 | 0.0159937 | 445 | 0.0035293 | 477 | 1 | 649 |
| Exemplary Red Channel A33 | 0.018909 | 445 | 0.0032024 | 485 | 1 | 645 |
| Exemplary Red Channel A34 | 0.036626 | 450 | 0.0045943 | 484 | 1 | 642 |
| Exemplary Red Channel A35 | 0.0391098 | 448 | 0.0046012 | 484 | 1 | 641 |
| Exemplary Red Channel A36 | 0.0121662 | 445 | 0.0020285 | 477 | 1 | 649 |
| Exemplary Red Channel A37 | 0.039642857 | 448 | 0.003571429 | 485 | 1 | 646 |
| Exemplary Red Channel A38 | 0.047058824 | 447 | 0.004080882 | 485 | 1 | 646 |
| Exemplary Red Channel A39 | 0.065408215 | 445 | 0.002961479 | 485 | 1 | 648 |
| Exemplary Red Channel A40 | 0.033147538 | 450 | 0.006545055 | 479 | 1 | 643 |
| Exemplary Red Channel A41 | 0.049912237 | 441 | 0.001980287 | 481 | 1 | 649 |
| Exemplary Red Channel A42 | 0.020176199 | 437 | 0.00091295 | 477 | 1 | 649 |
| Exemplary Red Channel A43 | 0.035129706 | 441 | 0.001239369 | 481 | 1 | 649 |
| Exemplary Red Channel A44 | 0.004557925 | 421 | 0.000132755 | 473 | 1 | 649 |
| Exemplary Red Channel A45 | 0.028092613 | 437 | 0.001190739 | 481 | 1 | 649 |
| Exemplary Red Channel A46 | 0.037872939 | 449 | 0.007765315 | 485 | 1 | 649 |
| Exemplary Red Channel A47 | 0.013726393 | 449 | 0.004099493 | 477 | 1 | 649 |
| Exemplary Red Channel A48 | 0.026683938 | 449 | 0.005008636 | 485 | 1 | 649 |
| Exemplary Red Channel A49 | 0.002054213 | 425 | 0.000669852 | 471 | 1 | 649 |
| Exemplary Red Channel A50 | 0.020574482 | 449 | 0.004898686 | 481 | 1 | 649 |
| Exemplary Red Channels | 0.002 | 421 | 0.000 | 471 | 1 | 640 |
| Minimum A1 | | | | | | |
| Exemplary Red Channels | 0.035 | 445 | 0.004 | 481 | 1 | 647 |
| Average A1 | | | | | | |
| Exemplary Red Channels | 0.184 | 451 | 0.011 | 497 | 1 | 653 |
| Maximum A1 | | | | | | |
| Exemplary Red Channels | 0.002 | 417 | 0.000 | 471 | 1 | 610 |
| Minimum A2 | | | | | | |
| Exemplary Red Channels | 0.075 | 446 | 0.009 | 484 | 1 | 643 |
| Average A2 | | | | | | |
| Exemplary Red Channels | 0.373 | 461 | 0.036 | 497 | 1 | 654 |
| Maximum A2 | | | | | | |

TABLE 20B

| | 400 < λ ≤ 470/ | 470 < λ ≤ 510/ | 530 < λ ≤ 570/ | 600 < λ ≤ 630/ | 630 < λ ≤ 780/ |
|---|---|---|---|---|---|
| | 470 < λ ≤ 510 | 470 < λ ≤ 510 | 470 < λ ≤ 510 | 470 < λ ≤ 510 | 470 < λ ≤ 510 |
| Exemplary Red Channel A1 | 1.4240434 | 1 | 1.9427142 | 15.938615 | 11.492902 |
| Exemplary Red Channel A2 | 2.0970522 | 1 | 4.0621523 | 28.921103 | 23.102883 |
| Exemplary Red Channel A3 | 5.1780425 | 1 | 4.0974298 | 26.293645 | 7.5734689 |
| Exemplary Red Channel A4 | 5.2381939 | 1 | 4.6168899 | 54.81712 | 25.89557 |
| Exemplary Red Channel A5 | 5.7161941 | 1 | 10.658471 | 40.602001 | 58.507947 |
| Exemplary Red Channel A6 | 1.5232234 | 1 | 3.3441154 | 11.765347 | 16.931963 |
| Exemplary Red Channel A7 | 12.048525 | 1 | 6.5531424 | 62.578621 | 209.5615 |
| Exemplary Red Channel A8 | 2.8231849 | 1 | 2.1009331 | 10.53909 | 37.837917 |
| Exemplary Red Channel A9 | 12.131662 | 1 | 6.1962328 | 38.56262 | 137.46985 |
| Exemplary Red Channel A10 | 0.9539982 | 1 | 4.3961077 | 13.215316 | 44.611756 |
| Exemplary Red Channel A11 | 0.9539982 | 1 | 4.3961077 | 13.215316 | 44.611756 |
| Exemplary Red Channel A12 | 4.6024544 | 1 | 9.5391075 | 43.065767 | 62.63258 |
| Exemplary Red Channel A13 | 398.70563 | 1 | 17.835096 | 869.65645 | 4459.1282 |
| Exemplary Red Channel A14 | 12.048525 | 1 | 6.5531424 | 62.578621 | 209.5615 |
| Exemplary Red Channel A15 | 0.9539982 | 1 | 4.3961077 | 13.215316 | 44.611756 |
| Exemplary Red Channel A16 | 12.048525 | 1 | 6.5531424 | 62.578621 | 209.5615 |
| Exemplary Red Channel A17 | 0.9539982 | 1 | 4.3961077 | 13.215316 | 44.611756 |
| Exemplary Red Channel A18 | 0.9539982 | 1 | 4.3961077 | 13.215316 | 44.611756 |
| Exemplary Red Channel A19 | 0.81649 | 1 | 4.0955819 | 12.540163 | 37.87807 |
| Exemplary Red Channel A20 | 0.9607978 | 1 | 4.4301483 | 13.507542 | 33.532199 |
| Exemplary Red Channel A21 | 12.048525 | 1 | 6.5531424 | 62.578621 | 209.5615 |
| Exemplary Red Channel A22 | 1.6208819 | 1 | 5.5397201 | 13.309323 | 58.491665 |
| Exemplary Red Channel A23 | 1.9869105 | 1 | 8.4014195 | 21.925049 | 78.866239 |
| Exemplary Red Channel A24 | 1.3138869 | 1 | 12.177683 | 39.15852 | 109.49194 |
| Exemplary Red Channel A25 | 1.2607323 | 1 | 12.636034 | 42.70977 | 120.6879 |
| Exemplary Red Channel A26 | 2.3890486 | 1 | 15.829408 | 56.787282 | 123.96913 |
| Exemplary Red Channel A27 | 2.5068143 | 1 | 15.724729 | 55.265962 | 118.90894 |
| Exemplary Red Channel A28 | 1.2607323 | 1 | 12.636034 | 42.70977 | 120.6879 |
| Exemplary Red Channel A29 | 1.2423614 | 1 | 9.9714945 | 29.913675 | 100.12202 |
| Exemplary Red Channel A30 | 2.5404104 | 1 | 18.56676 | 66.8566 | 145.35343 |
| Exemplary Red Channel A31 | 2.4685315 | 1 | 20.652257 | 66.891608 | 187.88207 |
| Exemplary Red Channel A32 | 1.2607323 | 1 | 12.636034 | 42.70977 | 120.6879 |
| Exemplary Red Channel A33 | 2.4718043 | 1 | 20.672747 | 66.961148 | 188.0788 |
| Exemplary Red Channel A34 | 2.4549564 | 1 | 15.396383 | 55.106075 | 120.34114 |
| Exemplary Red Channel A35 | 2.5421311 | 1 | 14.946486 | 52.254419 | 112.62205 |
| Exemplary Red Channel A36 | 0.9762043 | 1 | 13.498811 | 45.927857 | 133.58499 |
| Exemplary Red Channel A37 | 2.7811207 | 1 | 15.451912 | 49.917232 | 136.98072 |
| Exemplary Red Channel A38 | 2.8218352 | 1 | 14.28298 | 44.58311 | 122.43649 |
| Exemplary Red Channel A39 | 4.887349 | 1 | 16.854587 | 60.837387 | 170.39844 |
| Exemplary Red Channel A40 | 2.1214547 | 1 | 12.118194 | 42.691978 | 101.81216 |
| Exemplary Red Channel A41 | 2.7039225 | 1 | 14.218647 | 42.699353 | 114.4395 |
| Exemplary Red Channel A42 | 1.4190061 | 1 | 16.731611 | 49.510192 | 131.41829 |
| Exemplary Red Channel A43 | 3.5401364 | 1 | 18.671778 | 76.973836 | 217.50432 |
| Exemplary Red Channel A44 | 0.6871342 | 1 | 27.413538 | 110.76292 | 308.4831 |
| Exemplary Red Channel A45 | 2.3091088 | 1 | 17.504362 | 60.855688 | 166.58781 |
| Exemplary Red Channel A46 | 1.7603484 | 1 | 10.733065 | 32.2499 | 87.661419 |
| Exemplary Red Channel A47 | 0.9197238 | 1 | 13.515008 | 39.918863 | 107.32009 |
| Exemplary Red Channel A48 | 2.1700207 | 1 | 13.212248 | 54.386662 | 155.70883 |
| Exemplary Red Channel A49 | 0.3952841 | 1 | 23.031469 | 92.591647 | 260.678 |
| Exemplary Red Channel A50 | 1.4775171 | 1 | 13.324504 | 46.247693 | 128.24431 |
| Exemplary Red Channels | 0.395 | 1 | 5.540 | 13.309 | 58.492 |
| Minimum A1 | | | | | |
| Exemplary Red Channels | 2.345 | 1 | 14.763 | 52.176 | 141.967 |
| Average A1 | | | | | |
| Exemplary Red Channels | 12.049 | 1 | 27.414 | 110.763 | 308.483 |
| Maximum A1 | | | | | |
| Exemplary Red Channels | 0.395 | 1 | 1.943 | 10.539 | 7.573 |
| Minimum A2 | | | | | |
| Exemplary Red Channels | 11.049 | 1 | 11.149 | 59.706 | 200.455 |
| Average A2 | | | | | |
| Exemplary Red Channels | 398.706 | 1 | 27.414 | 869.656 | 4459.128 |
| Maximum A2 | | | | | |

TABLE 21A

| | Bp | | Bv | | Gp | | Rp | |
|---|---|---|---|---|---|---|---|---|
| | Bp | λ | Bv | λ | Gp | λ | Rp | λ |
| Exemplary Blue Channel A1 | 1 | 421 | 0.001566977 | 492 | 0.165696633 | 584 | 0.160100287 | 591 |
| Exemplary Blue Channel A2 | 1 | 420 | 0.00738 | 484 | 0.086087 | 544 | 0.101241 | 639 |
| Exemplary Blue Channel A3 | 1 | 449 | 0.018542 | 497 | 0.128604 | 600 | 0.138417 | 618 |
| Exemplary Blue Channel A4 | 1 | 420 | 0.120234 | 497 | 0.188421 | 527 | 0.113834 | 625 |
| Exemplary Blue Channel A5 | 1 | 453 | 0.046994 | 494 | 0.198491 | 544 | 0.150157 | 591 |
| Exemplary Blue Channel A6 | 1 | 449 | 0.022509 | 494 | 0.194339 | 601 | 0.198668 | 610 |
| Exemplary Blue Channel A7 | 1 | 461 | 0.073106359 | 509 | 0.200483818 | 601 | 0.20227698 | 608 |
| Exemplary Blue Channel A8 | 1 | 446 | 0.046208 | 485 | 0.233388 | 552 | 0.233331 | 631 |
| Exemplary Blue Channel A9 | 1 | 453 | 0.046994 | 494 | 0.198491 | 544 | 0.150157 | 591 |
| Exemplary Blue Channel A10 | 1 | 446 | 0.035402 | 486 | 0.173726 | 601 | 0.184844 | 614 |
| Exemplary Blue Channel A11 | 1 | 461 | 0.075839946 | 509 | 0.17126851 | 601 | 0.286585789 | 627 |
| Exemplary Blue Channel A12 | 1 | 446 | 0.064974 | 498 | 0.254044 | 601 | 0.272401 | 611 |
| Exemplary Blue Channel A13 | 1 | 420 | 0.01032 | 477 | 0.212974 | 601 | 0.222659 | 608 |
| Exemplary Blue Channel A14 | 1 | 449 | 0.018542 | 497 | 0.128604 | 600 | 0.138417 | 618 |
| Exemplary Blue Channel A15 | 1 | 461 | 0.075839946 | 509 | 0.17126851 | 601 | 0.286585789 | 627 |
| Exemplary Blue Channel A16 | 1 | 449 | 0.053176 | 495 | 0.190492 | 551 | 0.162643 | 591 |
| Exemplary Blue Channel A17 | 1 | 453 | 0.046994 | 494 | 0.198491 | 544 | 0.150157 | 591 |
| Exemplary Blue Channel A18 | 1 | 453 | 0.046994 | 494 | 0.198491 | 544 | 0.150157 | 591 |
| Exemplary Blue Channel A19 | 1 | 457 | 0.1621249 | 505 | 0.1776939 | 537 | 0.1497359 | 621 |
| Exemplary Blue Channel A20 | 1 | 453 | 0.046994 | 494 | 0.198491 | 544 | 0.150157 | 591 |
| Exemplary Blue Channel A21 | 1 | 452 | 0.169 | 491 | 0.179 | 535 | 0.145 | 593 |
| Exemplary Blue Channel A22 | 1 | 453 | 0.233252363 | 485 | 0.253211976 | 511 | 0.144546226 | 591 |
| Exemplary Blue Channel A23 | 1 | 449 | 0.139202 | 485 | 0.1721062 | 537 | 0.135281 | 591 |
| Exemplary Blue Channel A24 | 1 | 453 | 0.2667629 | 485 | 0.2843449 | 511 | 0.1518332 | 591 |
| Exemplary Blue Channel A25 | 1 | 450 | 0.211596858 | 490 | 0.220248698 | 531 | 0.181502027 | 593 |
| Exemplary Blue Channel A26 | 1 | 452 | 0.213563187 | 500 | 0.220511421 | 518 | 0.180127855 | 594 |
| Exemplary Blue Channel A27 | 1 | 453 | 0.2667629 | 485 | 0.2843449 | 511 | 0.1518332 | 591 |
| Exemplary Blue Channel A28 | 1 | 453 | 0.3437 | 485 | 0.355 | 511 | 0.1992 | 591 |
| Exemplary Blue Channel A29 | 1 | 453 | 0.3437 | 485 | 0.355 | 511 | 0.1992 | 591 |
| Exemplary Blue Channel A30 | 1 | 453 | 0.2667629 | 485 | 0.2843449 | 511 | 0.1518332 | 591 |
| Exemplary Blue Channel A31 | 1 | 453 | 0.2604551 | 481 | 0.2786344 | 511 | 0.1465555 | 591 |
| Exemplary Blue Channel A32 | 1 | 450 | 0.2115953 | 490 | 0.2202438 | 531 | 0.1814968 | 593 |
| Exemplary Blue Channel A33 | 1 | 452 | 0.2135661 | 500 | 0.2205124 | 518 | 0.1801269 | 594 |
| Exemplary Blue Channel A34 | 1 | 453 | 0.3437274 | 485 | 0.3550417 | 511 | 0.1991662 | 591 |
| Exemplary Blue Channel A35 | 1 | 449 | 0.289473684 | 482 | 0.325077399 | 514 | 0.1625387 | 591 |
| Exemplary Blue Channel A36 | 1 | 449 | 0.267669173 | 477 | 0.320300752 | 514 | 0.154887218 | 591 |
| Exemplary Blue Channel A37 | 1 | 450 | 0.302496955 | 483 | 0.338026792 | 517 | 0.165719975 | 591 |
| Exemplary Blue Channel A38 | 1 | 457 | 0.342312154 | 503 | 0.34641062 | 527 | 0.202629205 | 591 |
| Exemplary Blue Channel A39 | 1 | 445 | 0.22226044 | 473 | 0.358515345 | 511 | 0.168884164 | 591 |
| Exemplary Blue Channel A40 | 1 | 445 | 0.165158505 | 473 | 0.238116587 | 511 | 0.112445582 | 591 |
| Exemplary Blue Channel A41 | 1 | 445 | 0.213695533 | 473 | 0.34840651 | 511 | 0.186081694 | 591 |
| Exemplary Blue Channel A42 | 1 | 445 | 0.158225006 | 473 | 0.229297138 | 511 | 0.124129844 | 591 |
| Exemplary Blue Channel A43 | 1 | 445 | 0.189806479 | 473 | 0.293017651 | 511 | 0.14857872 | 591 |
| Exemplary Blue Channel A44 | 1 | 453 | 0.358256648 | 485 | 0.367563603 | 511 | 0.187221708 | 591 |
| Exemplary Blue Channel A45 | 1 | 453 | 0.242472372 | 511 | 0.242472372 | 511 | 0.126114463 | 591 |
| Exemplary Blue Channel A46 | 1 | 453 | 0.345461986 | 485 | 0.357925479 | 511 | 0.205610432 | 591 |
| Exemplary Blue Channel A47 | 1 | 453 | 0.233845197 | 511 | 0.233845197 | 511 | 0.138613161 | 591 |
| Exemplary Blue Channel A48 | 1 | 453 | 0.298606905 | 489 | 0.299977685 | 511 | 0.165163059 | 591 |
| Exemplary Blue Channels | 1 | 445 | 0.047 | 473 | 0.172 | 511 | 0.112 | 591 |
| Minimum A1 | | | | | | | | |
| Exemplary Blue Channels | 1 | 451 | 0.247 | 487 | 0.282 | 517 | 0.164 | 591 |
| Average A1 | | | | | | | | |
| Exemplary Blue Channels | 1 | 457 | 0.358 | 511 | 0.368 | 544 | 0.206 | 594 |
| Maximum A1 | | | | | | | | |
| Exemplary Blue Channels | 1 | 420 | 0.002 | 473 | 0.086 | 511 | 0.101 | 591 |
| Minimum A2 | | | | | | | | |
| Exemplary Blue Channels | 1 | 449 | 0.169 | 490 | 0.243 | 539 | 0.171 | 599 |
| Average A2 | | | | | | | | |
| Exemplary Blue Channels | 1 | 461 | 0.358 | 511 | 0.368 | 601 | 0.287 | 639 |
| Maximum A2 | | | | | | | | |

TABLE 21B

| | 400 < λ ≤ 470/ | 470 < λ ≤ 510/ | 530 < λ ≤ 570/ | 600 < λ ≤ 630/ | 630 < λ ≤ 780/ |
|---|---|---|---|---|---|
| | 470 < λ ≤ 510 | 470 < λ ≤ 510 | 470 < λ ≤ 510 | 470 < λ ≤ 510 | 470 < λ ≤ 510 |
| Exemplary Blue Channel A1 | 55.706293 | 1 | 14.738987 | 11.579494 | 18.032784 |
| Exemplary Blue Channel A2 | 38.946592 | 1 | 5.104892 | 4.2496462 | 10.287397 |
| Exemplary Blue Channel A3 | 11.540508 | 1 | 2.5477073 | 2.1395376 | 4.0082499 |
| Exemplary Blue Channel A4 | 5.0698884 | 1 | 0.7039356 | 0.4000738 | 0.731169 |
| Exemplary Blue Channel A5 | 6.0405662 | 1 | 2.1167284 | 0.9446664 | 1.0254653 |
| Exemplary Blue Channel A6 | 10.949515 | 1 | 2.0795799 | 2.8952065 | 3.9019097 |
| Exemplary Blue Channel A7 | 1.8245669 | 1 | 0.4569783 | 0.5488502 | 0.7355721 |
| Exemplary Blue Channel A8 | 7.2584857 | 1 | 3.0599265 | 2.2941949 | 5.0052801 |
| Exemplary Blue Channel A9 | 6.0405662 | 1 | 2.1167284 | 0.9446664 | 1.0254653 |
| Exemplary Blue Channel A10 | 9.1775798 | 1 | 1.8713726 | 2.3052493 | 3.2452362 |
| Exemplary Blue Channel A11 | 1.8974685 | 1 | 0.3895893 | 0.6951451 | 0.6697809 |
| Exemplary Blue Channel A12 | 4.5525837 | 1 | 1.4721701 | 1.3225108 | 1.0704571 |
| Exemplary Blue Channel A13 | 20.578521 | 1 | 5.7193033 | 4.6253653 | 3.5900643 |
| Exemplary Blue Channel A14 | 11.540508 | 1 | 2.5477073 | 2.1395376 | 4.0082499 |
| Exemplary Blue Channel A15 | 1.8333155 | 1 | 0.3712913 | 0.2644697 | 0.5119996 |
| Exemplary Blue Channel A16 | 5.6611036 | 1 | 1.7691953 | 0.8970934 | 1.0579819 |
| Exemplary Blue Channel A17 | 6.0405662 | 1 | 2.1167284 | 0.9446664 | 1.0254653 |
| Exemplary Blue Channel A18 | 6.0405662 | 1 | 2.1167284 | 0.9446664 | 1.0254653 |
| Exemplary Blue Channel A19 | 2.2857323 | 1 | 0.7272398 | 0.4790059 | 0.915801 |
| Exemplary Blue Channel A20 | 6.0405662 | 1 | 2.1167284 | 0.9446664 | 1.0254653 |
| Exemplary Blue Channel A21 | 2.7226506 | 1 | 0.9215352 | 0.5421797 | 0.8550838 |
| Exemplary Blue Channel A22 | 2.0974816 | 1 | 0.8980514 | 0.2961701 | 0.3634195 |
| Exemplary Blue Channel A23 | 3.1536214 | 1 | 1.0233939 | 0.5947274 | 0.9095376 |
| Exemplary Blue Channel A24 | 1.997302 | 1 | 0.8910344 | 0.2756185 | 0.3018547 |
| Exemplary Blue Channel A25 | 2.8881146 | 1 | 0.9124299 | 0.581909 | 1.0264725 |
| Exemplary Blue Channel A26 | 2.8804343 | 1 | 0.8814063 | 0.5480495 | 0.9363435 |
| Exemplary Blue Channel A27 | 1.997302 | 1 | 0.8910344 | 0.2756185 | 0.3018547 |
| Exemplary Blue Channel A28 | 1.9999526 | 1 | 0.8698547 | 0.2888531 | 0.3549441 |
| Exemplary Blue Channel A29 | 1.9999526 | 1 | 0.8698547 | 0.2888531 | 0.3549441 |
| Exemplary Blue Channel A30 | 1.997302 | 1 | 0.8910344 | 0.2756185 | 0.3018547 |
| Exemplary Blue Channel A31 | 1.9715817 | 1 | 0.8909029 | 0.2712729 | 0.2948165 |
| Exemplary Blue Channel A32 | 2.8881146 | 1 | 0.9124299 | 0.581909 | 1.0264725 |
| Exemplary Blue Channel A33 | 2.8804343 | 1 | 0.8814063 | 0.5480495 | 0.9363435 |
| Exemplary Blue Channel A34 | 1.9999598 | 1 | 0.8698517 | 0.2888707 | 0.3549534 |
| Exemplary Blue Channel A35 | 2.1771337 | 1 | 0.8982989 | 0.259757 | 0.2664142 |
| Exemplary Blue Channel A36 | 2.1670569 | 1 | 0.925149 | 0.2562314 | 0.2670194 |
| Exemplary Blue Channel A37 | 2.1382671 | 1 | 0.8926932 | 0.2500652 | 0.249674 |
| Exemplary Blue Channel A38 | 1.7146383 | 1 | 0.7741959 | 0.2806989 | 0.3251353 |
| Exemplary Blue Channel A39 | 2.0102394 | 1 | 0.986586 | 0.2516026 | 0.2345058 |
| Exemplary Blue Channel A40 | 2.8517053 | 1 | 0.9614982 | 0.2523714 | 0.2477096 |
| Exemplary Blue Channel A41 | 2.0855957 | 1 | 1.0290515 | 0.3451727 | 0.4139271 |
| Exemplary Blue Channel A42 | 2.982232 | 1 | 1.0022386 | 0.3549749 | 0.4469538 |
| Exemplary Blue Channel A43 | 2.4033026 | 1 | 0.9984852 | 0.3044638 | 0.3416735 |
| Exemplary Blue Channel A44 | 1.700538 | 1 | 0.8486765 | 0.2504132 | 0.2538167 |
| Exemplary Blue Channel A45 | 2.3654916 | 1 | 0.7974595 | 0.254721 | 0.2843984 |
| Exemplary Blue Channel A46 | 1.7523747 | 1 | 0.8794975 | 0.3335348 | 0.4167713 |
| Exemplary Blue Channel A47 | 2.4533497 | 1 | 0.8241511 | 0.3438405 | 0.4623156 |
| Exemplary Blue Channel A48 | 2.0090911 | 1 | 0.8428428 | 0.2985638 | 0.358606 |
| Exemplary Blue Channels | 1.701 | 1 | 0.774 | 0.250 | 0.235 |
| Minimum A1 | | | | | |
| Exemplary Blue Channels | 2.425 | 1 | 0.944 | 0.367 | 0.480 |
| Average A1 | | | | | |
| Exemplary Blue Channels | 6.041 | 1 | 2.117 | 0.945 | 1.026 |
| Maximum A1 | | | | | |
| Exemplary Blue Channels | 1.701 | 1 | 0.371 | 0.250 | 0.235 |
| Minimum A2 | | | | | |
| Exemplary Blue Channels | 5.902 | 1 | 1.654 | 1.068 | 1.579 |
| Average A2 | | | | | |
| Exemplary Blue Channels | 55.706 | 1 | 14.739 | 11.579 | 18.033 |
| Maximum A2 | | | | | |

TABLE 22A

| | Bp | λ | Bv | λ | Gp | λ | Rp | λ |
|---|---|---|---|---|---|---|---|---|
| Exemplary SBC Channel A1 | 0.068963 | 461 | 0.034922 | 485 | 1 | 561 | 0.8753 | 591 |
| Exemplary SBC Channel A2 | 0.134154 | 446 | 0.021507 | 485 | 1 | 552 | 0.872712 | 591 |
| Exemplary SBC Channel A3 | 0.084160289 | 419 | 0.011133536 | 476 | 1 | 601 | 1.074665618 | 608 |
| Exemplary SBC Channel A4 | 0.032735397 | 461 | 0.020960294 | 482 | 1 | 601 | 1.27518978 | 634 |
| Exemplary SBC Channel A5 | 0.097312 | 449 | 0.018809 | 476 | 1 | 544 | 0.898889 | 591 |
| Exemplary SBC Channel A6 | 0.139888 | 461 | 0.07686 | 482 | 1 | 576 | 0.983746 | 592 |
| Exemplary SBC Channel A7 | 0.02569 | 424 | 0.008269 | 471 | 1 | 544 | 0.8361 | 591 |
| Exemplary SBC Channel A8 | 0.426667718 | 449 | 0.007170711 | 497 | 1 | 601 | 2.318367498 | 640 |
| Exemplary SBC Channel A9 | 0.388198025 | 420 | 0.007610056 | 476 | 1 | 601 | 1.217024715 | 635 |
| Exemplary SBC Channel A10 | 0.699453552 | 450 | 0.005082 | 479 | 1 | 533 | 0.257923497 | 591 |
| Exemplary SBC Channel A11 | 0.189515 | 446 | 0.055231 | 471 | 1 | 544 | 0.750186 | 591 |
| Exemplary SBC Channel A12 | 0.236674 | 461 | 0.068362 | 491 | 1 | 561 | 0.865982 | 591 |
| Exemplary SBC Channel A13 | 0.388198025 | 420 | 0.007610056 | 476 | 1 | 601 | 1.217024715 | 635 |
| Exemplary SBC Channel A14 | 0.142763 | 446 | 0.040818 | 471 | 1 | 530 | 0.490104 | 591 |
| Exemplary SBC Channel A15 | 0.134498462 | 420 | 0.004214297 | 477 | 1 | 578 | 0.910844948 | 591 |
| Exemplary SBC Channel A16 | 0.032735397 | 461 | 0.020960294 | 482 | 1 | 601 | 1.27518978 | 634 |
| Exemplary SBC Channel A17 | 0.93012488 | 461 | 0.213852657 | 493 | 1 | 544 | 0.817090322 | 591 |
| Exemplary SBC Channel A18 | 0.042040823 | 461 | 0.030810256 | 479 | 1 | 601 | 1.385976413 | 640 |
| Exemplary SBC Channel A19 | 0.032735397 | 461 | 0.020960294 | 482 | 1 | 601 | 1.27518978 | 634 |
| Exemplary SBC Channel A20 | 0.032735397 | 461 | 0.020960294 | 482 | 1 | 601 | 1.27518978 | 634 |
| Exemplary SBC Channel A21 | 0.1712788 | 453 | 0.2026488 | 471 | 1 | 545 | 0.6757849 | 591 |
| Exemplary SBC Channel A22 | 0.1786135 | 453 | 0.2016162 | 471 | 1 | 545 | 0.7136418 | 591 |
| Exemplary SBC Channel A23 | 0.413844 | 458 | 0.253431 | 473 | 1 | 518 | 0.678419 | 591 |
| Exemplary SBC Channel A24 | 0.022778021 | 461 | 0.017050269 | 473 | 1 | 601 | 1.127976617 | 633 |
| Exemplary SBC Channel A25 | 0.022996001 | 461 | 0.016197691 | 473 | 1 | 601 | 1.115812401 | 641 |
| Exemplary SBC Channel A26 | 0.1712788 | 453 | 0.2026488 | 471 | 1 | 545 | 0.6757849 | 591 |
| Exemplary SBC Channel A27 | 0.1786135 | 453 | 0.2016162 | 471 | 1 | 545 | 0.7136418 | 591 |
| Exemplary SBC Channel A28 | 0.032735397 | 461 | 0.020960294 | 482 | 1 | 601 | 1.27518978 | 634 |
| Exemplary SBC Channel A29 | 0.015538291 | 454 | 0.011320755 | 471 | 1 | 600 | 1.109877913 | 647 |
| Exemplary SBC Channel A30 | 0.223 | 453 | 0.123 | 475 | 1 | 546 | 0.712 | 591 |
| Exemplary SBC Channel A31 | 0.1595974 | 453 | 0.1395072 | 471 | 1 | 533 | 0.5648081 | 591 |
| Exemplary SBC Channel A32 | 0.289151576 | 449 | 0.134054326 | 473 | 1 | 545 | 0.721244131 | 591 |
| Exemplary SBC Channel A33 | 0.361221522 | 449 | 0.183906932 | 471 | 1 | 549 | 0.753465826 | 591 |
| Exemplary SBC Channel A34 | 0.2010384 | 453 | 0.2085941 | 471 | 1 | 553 | 0.7377383 | 591 |
| Exemplary SBC Channel A35 | 0.03356814 | 449 | 0.006896908 | 481 | 1 | 597 | 1.177288319 | 645 |
| Exemplary SBC Channel A36 | 0.2679084 | 449 | 0.1563915 | 473 | 1 | 545 | 0.7311089 | 591 |
| Exemplary SBC Channel A37 | 0.011521957 | 429 | 0.008742499 | 471 | 1 | 601 | 1.11853883 | 645 |
| Exemplary SBC Channel A38 | 0.160952482 | 449 | 0.007588248 | 481 | 1 | 601 | 1.179398271 | 645 |
| Exemplary SBC Channel A39 | 0.109386089 | 449 | 0.114620298 | 471 | 1 | 548 | 0.74325763 | 591 |
| Exemplary SBC Channel A40 | 0.1206792 | 453 | 0.137608652 | 471 | 1 | 543 | 0.741484738 | 591 |
| Exemplary SBC Channel A41 | 0.2679084 | 449 | 0.1563915 | 473 | 1 | 545 | 0.7311089 | 591 |
| Exemplary SBC Channel A42 | 0.141616 | 453 | 0.1514584 | 471 | 1 | 549 | 0.7362628 | 591 |
| Exemplary SBC Channel A43 | 0.2679084 | 449 | 0.1563915 | 473 | 1 | 545 | 0.7311089 | 591 |
| Exemplary SBC Channel A44 | 0.2623323 | 453 | 0.1663439 | 473 | 1 | 549 | 0.7240392 | 591 |
| Exemplary SBC Channel A45 | 0.141616 | 453 | 0.1514584 | 471 | 1 | 549 | 0.7362628 | 591 |
| Exemplary SBC Channel A46 | 0.116859053 | 453 | 0.124270726 | 471 | 1 | 540 | 0.7224128 | 591 |
| Exemplary SBC Channel A47 | 0.201505897 | 458 | 0.194134372 | 471 | 1 | 540 | 0.723620472 | 591 |
| Exemplary SBC Channel A48 | 0.282304121 | 441 | 0.121779859 | 471 | 1 | 521 | 0.712313571 | 591 |
| Exemplary SBC Channel A49 | 0.159234893 | 441 | 0.091374269 | 471 | 1 | 521 | 0.717186485 | 591 |
| Exemplary SBC Channel A50 | 0.216999384 | 441 | 0.105179843 | 471 | 1 | 553 | 0.757365227 | 591 |
| Exemplary SBC Channel A51 | 0.097604298 | 441 | 0.070571416 | 471 | 1 | 553 | 0.759519993 | 591 |
| Exemplary SBC Channel A52 | 0.188152423 | 441 | 0.098786387 | 471 | 1 | 553 | 0.740049223 | 591 |
| Exemplary SBC Channel A53 | 0.249867068 | 461 | 0.249822758 | 471 | 1 | 521 | 0.721065225 | 591 |
| Exemplary SBC Channel A54 | 0.147124231 | 461 | 0.179435748 | 471 | 1 | 521 | 0.72376052 | 591 |
| Exemplary SBC Channel A55 | 0.194807647 | 461 | 0.212556054 | 471 | 1 | 553 | 0.763039887 | 591 |
| Exemplary SBC Channel A56 | 0.091726119 | 461 | 0.133327156 | 471 | 1 | 553 | 0.763365144 | 591 |
| Exemplary SBC Channel A57 | 0.171172401 | 461 | 0.196795922 | 471 | 1 | 553 | 0.745448753 | 591 |
| Exemplary SBC Channels | 0.012 | 429 | 0.007 | 471 | 1 | 521 | 0.565 | 591 |
| Minimum A1 | | | | | | | | |
| Exemplary SBC Channels | 0.173 | 451 | 0.127 | 473 | 1 | 553 | 0.802 | 600 |
| Average A1 | | | | | | | | |
| Exemplary SBC Channels | 0.361 | 461 | 0.250 | 482 | 1 | 601 | 1.275 | 647 |
| Maximum A1 | | | | | | | | |
| Exemplary SBC Channels | 0.012 | 419 | 0.004 | 471 | 1 | 518 | 0.258 | 591 |
| Minimum A2 | | | | | | | | |
| Exemplary SBC Channels | 0.186 | 450 | 0.098 | 475 | 1 | 561 | 0.889 | 604 |
| Average A2 | | | | | | | | |
| Exemplary SBC Channels | 0.930 | 461 | 0.253 | 497 | 1 | 601 | 2.318 | 647 |
| Maximum A2 | | | | | | | | |

TABLE 22B

| | 400 < λ ≤ 470/ | 470 < λ ≤ 510/ | 530 < λ ≤ 570/ | 600 < λ ≤ 630/ | 630 < λ ≤ 780/ |
|---|---|---|---|---|---|
| | 470 < λ ≤ 510 | 470 < λ ≤ 510 | 470 < λ ≤ 510 | 470 < λ ≤ 510 | 470 < λ ≤ 510 |
| Exemplary SBC Channel A1 | 0.4390569 | 1 | 9.7228651 | 5.7900278 | 4.9878394 |
| Exemplary SBC Channel A2 | 0.6610607 | 1 | 6.8709886 | 3.9693561 | 3.8075052 |
| Exemplary SBC Channel A3 | 0.707303 | 1 | 6.4476935 | 6.0435463 | 4.2311708 |
| Exemplary SBC Channel A4 | 0.2236308 | 1 | 10.978042 | 12.168252 | 27.48015 |
| Exemplary SBC Channel A5 | 0.4617927 | 1 | 6.7495755 | 3.683658 | 4.0953561 |
| Exemplary SBC Channel A6 | 0.4045256 | 1 | 5.5396668 | 3.7634919 | 4.6005166 |
| Exemplary SBC Channel A7 | 0.1341095 | 1 | 6.7296204 | 3.9396543 | 7.3372636 |
| Exemplary SBC Channel A8 | 12.048525 | 1 | 6.5531424 | 62.578621 | 209.5615 |
| Exemplary SBC Channel A9 | 1.9299417 | 1 | 6.6782889 | 6.4211117 | 15.357954 |
| Exemplary SBC Channel A10 | 1.4190661 | 1 | 3.2651287 | 0.2820189 | 0.0964278 |
| Exemplary SBC Channel A11 | 0.3147168 | 1 | 2.7848358 | 1.3668204 | 2.3510841 |
| Exemplary SBC Channel A12 | 0.8264894 | 1 | 6.8132332 | 3.7526 | 3.8552956 |
| Exemplary SBC Channel A13 | 1.9299417 | 1 | 6.6782889 | 6.4211117 | 15.357954 |
| Exemplary SBC Channel A14 | 0.2415859 | 1 | 2.4397529 | 1.0300744 | 1.3184598 |
| Exemplary SBC Channel A15 | 0.4850942 | 1 | 6.1064732 | 3.7229416 | 4.6499052 |
| Exemplary SBC Channel A16 | 0.2236308 | 1 | 10.978042 | 12.168252 | 27.48015 |
| Exemplary SBC Channel A17 | 1.2492775 | 1 | 2.562718 | 1.4000367 | 2.4220632 |
| Exemplary SBC Channel A18 | 0.2586453 | 1 | 6.5310069 | 7.1967565 | 18.464502 |
| Exemplary SBC Channel A19 | 0.2236308 | 1 | 10.978042 | 12.168252 | 27.48015 |
| Exemplary SBC Channel A20 | 0.2236308 | 1 | 10.978042 | 12.168252 | 27.48015 |
| Exemplary SBC Channel A21 | 0.1954326 | 1 | 1.5827163 | 0.7079335 | 1.4843123 |
| Exemplary SBC Channel A22 | 0.2051631 | 1 | 1.6136566 | 0.7044634 | 1.1235442 |
| Exemplary SBC Channel A23 | 0.3983781 | 1 | 1.510326 | 0.7873699 | 1.2071515 |
| Exemplary SBC Channel A24 | 0.1383596 | 1 | 8.0262241 | 9.1378601 | 20.935409 |
| Exemplary SBC Channel A25 | 0.1548541 | 1 | 8.1932903 | 9.2061564 | 17.054853 |
| Exemplary SBC Channel A26 | 0.1954326 | 1 | 1.5827163 | 0.7079335 | 1.4843123 |
| Exemplary SBC Channel A27 | 0.2051631 | 1 | 1.6136566 | 0.7044634 | 1.1235442 |
| Exemplary SBC Channel A28 | 0.2236308 | 1 | 10.978042 | 12.168252 | 27.48015 |
| Exemplary SBC Channel A29 | 0.213027 | 1 | 10.297866 | 10.699754 | 22.221442 |
| Exemplary SBC Channel A30 | 0.3917323 | 1 | 2.7293852 | 1.0586058 | 1.4544369 |
| Exemplary SBC Channel A31 | 0.2133805 | 1 | 1.8198726 | 0.4729991 | 0.471368 |
| Exemplary SBC Channel A32 | 0.3935947 | 1 | 1.992482 | 0.8200205 | 1.227975 |
| Exemplary SBC Channel A33 | 0.4099694 | 1 | 1.7860828 | 0.8264234 | 1.3437975 |
| Exemplary SBC Channel A34 | 0.2627506 | 1 | 1.8554004 | 0.7592516 | 1.1352777 |
| Exemplary SBC Channel A35 | 0.3809845 | 1 | 13.289798 | 13.7393 | 30.274673 |
| Exemplary SBC Channel A36 | 0.4083884 | 1 | 2.0662521 | 0.8690062 | 1.2754065 |
| Exemplary SBC Channel A37 | 0.2326486 | 1 | 11.506186 | 12.682358 | 26.464523 |
| Exemplary SBC Channel A38 | 1.6579291 | 1 | 13.071521 | 13.54784 | 29.884202 |
| Exemplary SBC Channel A39 | 0.2380303 | 1 | 1.9957951 | 0.8611959 | 1.2111385 |
| Exemplary SBC Channel A40 | 0.2533932 | 1 | 2.0256628 | 0.8715624 | 1.1783715 |
| Exemplary SBC Channel A41 | 0.4083884 | 1 | 2.0662521 | 0.8690062 | 1.2754065 |
| Exemplary SBC Channel A42 | 0.2828711 | 1 | 1.8440184 | 0.7562172 | 1.0210213 |
| Exemplary SBC Channel A43 | 0.4083884 | 1 | 2.0662521 | 0.8690062 | 1.2754065 |
| Exemplary SBC Channel A44 | 0.3617499 | 1 | 1.9600988 | 0.8062245 | 1.1926305 |
| Exemplary SBC Channel A45 | 0.2828711 | 1 | 1.8440184 | 0.7562172 | 1.0210213 |
| Exemplary SBC Channel A46 | 0.2510099 | 1 | 2.030957 | 0.8410699 | 1.1053413 |
| Exemplary SBC Channel A47 | 0.358773 | 1 | 2.0794223 | 0.8648344 | 1.1269186 |
| Exemplary SBC Channel A48 | 0.5440345 | 1 | 1.9979444 | 0.747063 | 0.8660678 |
| Exemplary SBC Channel A49 | 0.3570619 | 1 | 2.069514 | 0.7796073 | 0.91017 |
| Exemplary SBC Channel A50 | 0.4607363 | 1 | 2.107838 | 0.9042748 | 1.2135833 |
| Exemplary SBC Channel A51 | 0.2615933 | 1 | 2.2052887 | 0.9484459 | 1.272289 |
| Exemplary SBC Channel A52 | 0.4076762 | 1 | 2.0880147 | 0.8403135 | 1.0579656 |
| Exemplary SBC Channel A53 | 0.3948592 | 1 | 1.7622443 | 0.679278 | 0.8252617 |
| Exemplary SBC Channel A54 | 0.2470896 | 1 | 1.8580271 | 0.716574 | 0.8687501 |
| Exemplary SBC Channel A55 | 0.3257219 | 1 | 1.866986 | 0.8210642 | 1.1406785 |
| Exemplary SBC Channel A56 | 0.1699051 | 1 | 1.9988583 | 0.8750537 | 1.2064352 |
| Exemplary SBC Channel A57 | 0.2855385 | 1 | 1.8618528 | 0.7676357 | 1.001879 |
| Exemplary SBC Channels | 0.170 | 1 | 1.762 | 0.473 | 0.471 |
| Minimum A1 | | | | | |
| Exemplary SBC Channels | 0.370 | 1 | 3.637 | 2.774 | 5.467 |
| Average A1 | | | | | |
| Exemplary SBC Channels | 1.658 | 1 | 13.290 | 13.739 | 30.275 |
| Maximum A1 | | | | | |
| Exemplary SBC Channels | 0.134 | 1 | 1.510 | 0.282 | 0.096 |
| Minimum A2 | | | | | |
| Exemplary SBC Channels | 0.649 | 1 | 4.730 | 4.828 | 10.892 |
| Average A2 | | | | | |
| Exemplary SBC Channels | 12.049 | 1 | 13.290 | 62.579 | 209.562 |
| Maximum A2 | | | | | |

TABLE 23A

| | Cp | wavelength | Gv | wavelength | Rp | wavelength |
|---|---|---|---|---|---|---|
| Exemplary LBC Channel A1 | 1 | 506 | 0.651345 | 581 | 0.587996 | 591 |
| Exemplary LBC Channel A2 | 1 | 503 | 0.35416 | 581 | 0.348457 | 603 |
| Exemplary LBC Channel A3 | 1 | 501 | 0.390007 | 581 | 0.361221 | 591 |
| Exemplary LBC Channel A4 | 1 | 501 | 0.201994 | 560 | 0.289653 | 610 |
| Exemplary LBC Channel A5 | 1 | 503 | 0.480378 | 580 | 0.480884 | 581 |
| Exemplary LBC Channel A6 | 1 | 504 | 0.445745 | 580 | 0.390619 | 591 |
| Exemplary LBC Channel A7 | 1 | 501 | 0.156061 | 600 | 0.15975 | 610 |
| Exemplary LBC Channel A8 | 1 | 491 | 0.215477 | 534 | 0.209924 | 592 |
| Exemplary LBC Channel A9 | 1 | 502 | 0.435329 | 580 | 0.450946 | 618 |
| Exemplary LBC Channel A10 | 1 | 503 | 0.697479 | 601 | 0.719165 | 591 |
| Exemplary LBC Channel A11 | 1 | 506 | 0.0124102 | 535 | 0.0542129 | 591 |
| Exemplary LBC Channel A12 | 1 | 504 | 0.445745 | 580 | 0.390619 | 591 |
| Exemplary LBC Channel A13 | 1 | 502 | 0.446112 | 580 | 0.450946 | 618 |
| Exemplary LBC Channel A14 | 1 | 504 | 0.445745 | 580 | 0.390619 | 591 |
| Exemplary LBC Channel A15 | 1 | 502 | 0.446112 | 580 | 0.450946 | 618 |
| Exemplary LBC Channel A16 | 1 | 501 | 0.156061 | 600 | 0.15975 | 610 |
| Exemplary LBC Channel A17 | 1 | 518 | 0.707776 | 581 | 0.678419 | 591 |
| Exemplary LBC Channel A18 | 1 | 503 | 0.480378 | 580 | 0.480884 | 581 |
| Exemplary LBC Channel A19 | 1 | 478 | 0.502669 | 581 | 0.508747 | 591 |
| Exemplary LBC Channel A20 | 1 | 478 | 0.386028 | 581 | 0.347062 | 591 |
| Exemplary LBC Channel A21 | 1 | 496 | 0.37786 | 581 | 0.340149 | 591 |
| Exemplary LBC Channel A22 | 1 | 504 | 0.445745 | 580 | 0.390619 | 591 |
| Exemplary LBC Channel A23 | 1 | 477 | 0.386648571 | 581 | 0.344928331 | 591 |
| Exemplary LBC Channel A24 | 1 | 481 | 0.4295801 | 581 | 0.3743768 | 591 |
| Exemplary LBC Channel A25 | 1 | 481 | 0.4592037 | 581 | 0.3947325 | 591 |
| Exemplary LBC Channel A26 | 1 | 481 | 0.4592037 | 581 | 0.3947325 | 591 |
| Exemplary LBC Channel A27 | 1 | 476 | 0.3177314 | 581 | 0.2801215 | 591 |
| Exemplary LBC Channel A28 | 1 | 476 | 0.3177314 | 581 | 0.2801215 | 591 |
| Exemplary LBC Channel A29 | 1 | 483 | 0.442426943 | 581 | 0.380305224 | 591 |
| Exemplary LBC Channel A30 | 1 | 485 | 0.313274279 | 581 | 0.284036203 | 591 |
| Exemplary LBC Channel A31 | 1 | 485 | 0.263031949 | 581 | 0.246378218 | 591 |
| Exemplary LBC Channel A32 | 1 | 485 | 0.3704504 | 581 | 0.3256344 | 591 |
| Exemplary LBC Channel A33 | 1 | 480 | 0.4247818 | 581 | 0.3705277 | 591 |
| Exemplary LBC Channel A34 | 1 | 481 | 0.390243902 | 581 | 0.336759582 | 591 |
| Exemplary LBC Channel A35 | 1 | 485 | 0.481927711 | 581 | 0.415662651 | 591 |
| Exemplary LBC Channel A36 | 1 | 481 | 0.449612403 | 581 | 0.397286822 | 591 |
| Exemplary LBC Channel A37 | 1 | 485 | 0.563636364 | 581 | 0.497727273 | 591 |
| Exemplary LBC Channel A38 | 1 | 481 | 0.434456929 | 581 | 0.375842697 | 591 |
| Exemplary LBC Channel A39 | 1 | 481 | 0.332247557 | 581 | 0.291042345 | 591 |
| Exemplary LBC Channel A40 | 1 | 485 | 0.411985019 | 581 | 0.358426966 | 591 |
| Exemplary LBC Channel A41 | 1 | 481 | 0.381818182 | 581 | 0.345454545 | 591 |
| Exemplary LBC Channel A42 | 1 | 485 | 0.484398977 | 581 | 0.433418585 | 591 |
| Exemplary LBC Channel A43 | 1 | 481 | 0.36971831 | 581 | 0.327816901 | 591 |
| Exemplary LBC Channel A44 | 1 | 481 | 0.290288897 | 581 | 0.258646331 | 591 |
| Exemplary LBC Channel A45 | 1 | 485 | 0.357068663 | 581 | 0.314552813 | 591 |
| Exemplary LBC Channel A46 | 1 | 485 | 0.33042329 | 581 | 0.30259817 | 591 |
| Exemplary LBC Channel A47 | 1 | 489 | 0.419054587 | 581 | 0.379709791 | 591 |
| Exemplary LBC Channel A48 | 1 | 485 | 0.319780368 | 581 | 0.287172405 | 591 |
| Exemplary LBC Channel A49 | 1 | 485 | 0.250440154 | 581 | 0.228355212 | 591 |
| Exemplary LBC Channel A50 | 1 | 489 | 0.3127045 | 581 | 0.280406713 | 591 |
| Exemplary LBC Channel A51 | 1 | 485 | 0.28654931 | 581 | 0.267790389 | 591 |
| Exemplary LBC Channel A52 | 1 | 489 | 0.362721986 | 581 | 0.33372623 | 591 |
| Exemplary LBC Channel A53 | 1 | 485 | 0.277554384 | 581 | 0.254482531 | 591 |
| Exemplary LBC Channel A54 | 1 | 485 | 0.220707166 | 581 | 0.206946273 | 591 |
| Exemplary LBC Channel A55 | 1 | 489 | 0.272267206 | 581 | 0.249441575 | 591 |
| Exemplary LBC Channel A56 | 1 | 489 | 0.254319878 | 581 | 0.243641139 | 591 |
| Exemplary LBC Channel A57 | 1 | 489 | 0.31884909 | 581 | 0.298806029 | 591 |
| Exemplary LBC Channel A58 | 1 | 489 | 0.246105919 | 581 | 0.231333534 | 591 |
| Exemplary LBC Channels | 1 | 476 | 0.221 | 580 | 0.207 | 591 |
| Minimum A1 | | | | | | |
| Exemplary LBC Channels | 1 | 484 | 0.363 | 581 | 0.324 | 591 |
| Average A1 | | | | | | |
| Exemplary LBC Channels | 1 | 504 | 0.564 | 581 | 0.498 | 591 |
| Maximum A1 | | | | | | |
| Exemplary LBC Channels | 1 | 476 | 0.012 | 534 | 0.054 | 581 |
| Minimum A2 | | | | | | |
| Exemplary LBC Channels | 1 | 490 | 0.377 | 580 | 0.349 | 593 |
| Average A2 | | | | | | |
| Exemplary LBC Channels | 1 | 518 | 0.708 | 601 | 0.719 | 650 |
| Maximum A2 | | | | | | |

TABLE 23B

| | 400 < λ ≤ 470/ | 470 < λ ≤ 510/ | 530 < λ ≤ 570/ | 600 < λ ≤ 630/ | 630 < λ ≤ 780/ |
|---|---|---|---|---|---|
| | 470 < λ ≤ 510 | 470 < λ ≤ 510 | 470 < λ ≤ 510 | 470 < λ ≤ 510 | 470 < λ ≤ 510 |
| Exemplary LBC Channel A1 | 0.0571188 | 1 | 1.3707541 | 0.4997694 | 0.4842527 |
| Exemplary LBC Channel A2 | 0.0416035 | 1 | 0.7188973 | 0.3932214 | 0.3757458 |
| Exemplary LBC Channel A3 | 0.0425228 | 1 | 0.7685004 | 0.3851276 | 0.3064902 |
| Exemplary LBC Channel A4 | 0.0472241 | 1 | 0.3880302 | 0.3390386 | 0.4545081 |
| Exemplary LBC Channel A5 | 0.0401666 | 1 | 0.8752875 | 0.4664636 | 0.5587806 |
| Exemplary LBC Channel A6 | 0.0391102 | 1 | 0.9074872 | 0.3246497 | 0.4085832 |
| Exemplary LBC Channel A7 | 0.0451544 | 1 | 0.386109 | 0.19085 | 0.3393066 |
| Exemplary LBC Channel A8 | 0.0466565 | 1 | 0.3803029 | 0.2501522 | 0.4608856 |
| Exemplary LBC Channel A9 | 0.044065 | 1 | 0.8288683 | 0.5439467 | 1.11703 |
| Exemplary LBC Channel A10 | 0.0446968 | 1 | 1.3114156 | 0.830796 | 1.3370571 |
| Exemplary LBC Channel A11 | 0 | 1 | 0.1317523 | 0.0863652 | 0.1171237 |
| Exemplary LBC Channel A12 | 0.0391102 | 1 | 0.9074872 | 0.3246497 | 0.4085832 |
| Exemplary LBC Channel A13 | 0.044065 | 1 | 0.8288683 | 0.5439467 | 1.11703 |
| Exemplary LBC Channel A14 | 0.0391102 | 1 | 0.9074872 | 0.3246497 | 0.4085832 |
| Exemplary LBC Channel A15 | 0.044065 | 1 | 0.8288683 | 0.5439467 | 1.11703 |
| Exemplary LBC Channel A16 | 0.0451544 | 1 | 0.386109 | 0.19085 | 0.3393066 |
| Exemplary LBC Channel A17 | 0.3983781 | 1 | 1.510326 | 0.7873699 | 1.2071515 |
| Exemplary LBC Channel A18 | 0.0401666 | 1 | 0.8752875 | 0.4664636 | 0.5587806 |
| Exemplary LBC Channel A19 | 0.2180645 | 1 | 0.7229216 | 0.4530321 | 0.5570927 |
| Exemplary LBC Channel A20 | 0.2174801 | 1 | 0.6858138 | 0.2392448 | 0.2451605 |
| Exemplary LBC Channel A21 | 0.072958 | 1 | 0.6766421 | 0.2289306 | 0.2372285 |
| Exemplary LBC Channel A22 | 0.0391102 | 1 | 0.9074872 | 0.3246497 | 0.4085832 |
| Exemplary LBC Channel A23 | 0.2202015 | 1 | 0.687311 | 0.2391496 | 0.2456338 |
| Exemplary LBC Channel A24 | 0.2041085 | 1 | 0.6386386 | 0.2240144 | 0.2177686 |
| Exemplary LBC Channel A25 | 0.2441363 | 1 | 0.6974219 | 0.2405667 | 0.2623651 |
| Exemplary LBC Channel A26 | 0.2441363 | 1 | 0.6974219 | 0.2405667 | 0.2623651 |
| Exemplary LBC Channel A27 | 0.1748609 | 1 | 0.5532819 | 0.2003928 | 0.1839335 |
| Exemplary LBC Channel A28 | 0.1748609 | 1 | 0.5532819 | 0.2003928 | 0.1839335 |
| Exemplary LBC Channel A29 | 0.2065643 | 1 | 0.6455041 | 0.2201597 | 0.2285261 |
| Exemplary LBC Channel A30 | 0.16755 | 1 | 0.5062788 | 0.211271 | 0.22.6348 |
| Exemplary LBC Channel A31 | 0.1129865 | 1 | 0.421533 | 0.2032093 | 0.2299145 |
| Exemplary LBC Channel A32 | 0.2046695 | 1 | 0.5587588 | 0.2083974 | 0.2264195 |
| Exemplary LBC Channel A33 | 0.234042 | 1 | 0.6218959 | 0.2215126 | 0.23109 |
| Exemplary LBC Channel A34 | 0.3236629 | 1 | 0.6185476 | 0.2005553 | 0.1969621 |
| Exemplary LBC Channel A35 | 0.2089596 | 1 | 0.7200383 | 0.2291225 | 0.2216762 |
| Exemplary LBC Channel A36 | 0.2867732 | 1 | 0.6621235 | 0.2554298 | 0.2735013 |
| Exemplary LBC Channel A37 | 0.1669428 | 1 | 0.7877293 | 0.3002941 | 0.3179063 |
| Exemplary LBC Channel A38 | 0.2815764 | 1 | 0.6582097 | 0.2272.843 | 0.2307814 |
| Exemplary LBC Channel A39 | 0.2.57809 | 1 | 0.5440018 | 0.191975 | 0.1978422 |
| Exemplary LBC Channel A40 | 0.16294 | 1 | 0.6464553 | 0.2198367 | 0.2211549 |
| Exemplary LBC Channel A41 | 0.2266279 | 1 | 0.5858923 | 0.2424262 | 0.2682179 |
| Exemplary LBC Channel A42 | 0.1279336 | 1 | 0.7134898 | 0.2867346 | 0.3113052 |
| Exemplary LBC Channel A43 | 0.2229097 | 1 | 0.5832721 | 0.2170375 | 0.2292726 |
| Exemplary LBC Channel A44 | 0.2029163 | 1 | 0.4762425 | 0.1851969 | 0.2006454 |
| Exemplary LBC Channel A45 | 0.1244013 | 1 | 0.5786317 | 0.2123714 | 0.2226371 |
| Exemplary LBC Channel A46 | 0.1767702 | 1 | 0.5154779 | 0.2318155 | 0.2655981 |
| Exemplary LBC Channel A47 | 0.095579 | 1 | 0.6431204 | 0.275141 | 0.3071361 |
| Exemplary LBC Channel A48 | 0.1736583 | 1 | 0.5136297 | 0.2085304 | 0.2296014 |
| Exemplary LBC Channel A49 | 0.1586816 | 1 | 0.4144812 | 0.1804485 | 0.2056122 |
| Exemplary LBC Channel A50 | 0.0938414 | 1 | 0.5150858 | 0.2067553 | 0.226456 |
| Exemplary LBC Channel A51 | 0.1366787 | 1 | 0.4514609 | 0.2235964 | 0.2657759 |
| Exemplary LBC Channel A52 | 0.0701879 | 1 | 0.5773825 | 0.2659013 | 0.3059702 |
| Exemplary LBC Channel A53 | 0.1342953 | 1 | 0.4504921 | 0.2022643 | 0.2325118 |
| Exemplary LBC Channel A54 | 0.1244724 | 1 | 0.3585474 | 0.1778658 | 0.2131465 |
| Exemplary LBC Channel A55 | 0.0705681 | 1 | 0.4563433 | 0.203288 | 0.2330024 |
| Exemplary LBC Channel A56 | 0.1058427 | 1 | 0.3933056 | 0.218043 | 0.2692776 |
| Exemplary LBC Channel A57 | 0.0511767 | 1 | 0.5161309 | 0.2592232 | 0.3082101 |
| Exemplary LBC Channel A58 | 0.1039876 | 1 | 0.3929967 | 0.1983506 | 0.2383038 |
| Exemplary LBC Channels | 0.039 | 1 | 0.359 | 0.178 | 0.184 |
| Minimum A1 | | | | | |
| Exemplary LBC Channels | 0.171 | 1 | 0.575 | 0.226 | 0.246 |
| Average A1 | | | | | |
| Exemplary LBC Channels | 0.324 | 1 | 0.907 | 0.325 | 0.409 |
| Maximum A1 | | | | | |
| Exemplary LBC Channels | 0.000 | 1 | 0.132 | 0.086 | 0.117 |
| Minimum A2 | | | | | |
| Exemplary LBC Channels | 0.137 | 1 | 0.649 | 0.289 | 0.366 |
| Average A2 | | | | | |
| Exemplary LBC Channels | 0.398 | 1 | 1.510 | 0.831 | 1.337 |
| Maximum A2 | | | | | |

What is claimed is:

1. A tunable lighting system comprising:
a plurality of channels comprising at least,
a first channel for emitting blue light and having a wavelength peak between 420 nm and 480 nm;
a second channel for emitting cyan light having a wavelength peak between 450 nm and 530 nm;
a third channel for emitting cyan-green light having a wavelength peak between 510 nm and 590 nm;
a fourth channel for emitting red light having a wavelength peak between 510 nm and 780 nm; and
a multichannel driver for driving a selection of said plurality of channels, said multichannel driver is configured to drive each channel independently such that said light system emits an emitted light with a CRI of at least 85 over a CCT range greater than 3000 K.

2. The tunable lighting system of claim 1, wherein said first channel emits unsaturated light having a spectral power distribution having a blue peak, a blue valley, a green peak, and a red peak.

3. The tunable lighting system of claim 2, wherein said blue peak is between 445 nm and 460 nm, the blue valley is between 470 nm and 515 nm, the green peak occurs at a wavelength between 510 nm and 605 nm, and the red peak occurs at a wavelength between 585 nm and 640 nm.

4. The tunable lighting system of claim 3, wherein the relative spectral power distribution ratio for wavelengths (400<λ≤470)/(470<λ≤510) is between about 1.6 and about 60, wherein the relative spectral power distribution ratio for wavelengths (530<λ≤570)/(470<λ≤510) is between about 0.37 and about 6.0, wherein the relative spectral power distribution ratio for wavelengths (600<λ≤630)/(470<λ≤510) is between about 0.25 and about 5.0, and wherein the relative spectral power distribution ratio for wavelengths (630<λ≤780)/(470<λ≤510) is between about 0.23 and about 1.5.

5. The tunable lighting system of claim 1, wherein said second channel emits unsaturated light having a spectral power distribution having a cyan peak, a green valley, and a red peak.

6. The tunable lighting system of claim 5, wherein the cyan peak occurs at a wavelength between 470 nm and 520 nm, wherein the green valley occurs at a wavelength between 530 nm and 550 nm, wherein the red peak occurs at a wavelength between 590 nm and 650 nm.

7. The tunable lighting system of claim 6, wherein the relative spectral power distribution ratio for wavelengths (400<λ≤470)/(470<λ≤510) is between about 0.04 and about 0.4, wherein the relative spectral power distribution ratio for wavelengths (530<λ≤570)/(470<λ≤510) is between about 0.13 and about 1.5, wherein the relative spectral power distribution ratio for wavelengths (600<λ≤630)/(470<λ≤510) is between about 0.08 and about 0,85, and wherein the relative spectral power distribution ratio for wavelengths (630<k 780)/(470<λ≤510) is between about 0.11 and about 1.4.

8. The tunable lighting system of claim 1, wherein said third channel emits unsaturated light having a spectral power distribution having a blue peak, a blue valley, a green peak, and a red peak.

9. The tunable lighting system of claim 8, wherein the blue peak occurs at a wavelength between 420 nm and 460 nm, wherein the blue valley occurs at a wavelength between 460 nm and 480 nm, the green peak occurs at a wavelength between 515 nm and 605 nm, and the red peak occurs at a wavelength between 590 nm and 650 nm.

10. The tunable lighting system of claim 9, wherein the relative spectral power distribution ratio for wavelengths (400<λ≤470)/(470<λ≤10) is between about 0.1 and about 12, wherein the relative spectral power distribution ratio for wavelengths (530<λ≤570)/(470<λ≤510) is between about 1.5 and about 5.0, wherein the relative spectral power distribution ratio for wavelengths (600<λ≤630)/(470<λ≤510) is between about 0,4 and about 15, wherein the relative spectral power distribution ratio for wavelengths (630<λ≤780)/(470<λ≤510) is between about 0.1 and about 30.

11. The tunable lighting system of claim 1, wherein said fourth channel emits unsaturated light having a spectral power distribution having a blue peak, a blue valley, and a red peak.

12. The tunable lighting system of claim 11, wherein the blue peak occurs at a wavelength between 420 nm and 465 nm, the blue valley occurs at a wavelength between 470 nm and 505 nm, and the red peak occurs at a wavelength between 610 nm and 660 nm. 13, The tunable lighting system of claim 12, wherein the relative spectral power distribution ratio for wavelengths (400<λ≤470)/(470<λ≤510) is between about 0.4 and about 15, wherein the relative spectral power distribution ratio for wavelengths (530<λ≤570)/(470<λ≤510) is between about 12 and about 18, wherein the relative spectral power distribution ratio for wavelengths (600<λ≤630)/(470<λ≤510) is between about 15 and about 1, and wherein the relative spectral power distribution ratio for wavelengths (630<λ≤780)/(470<λ≤510) is between about 55 and about 3.

14. The tunable lighting system of claim 1, further comprising:
a fifth channel for emitting violet light having a wavelength peak between 360 nm and 460 nm.

15. The tunable lighting system of claim 14, further comprising:
a sixth channel for emitting yellow light having a wavelength peak between 510 nm and 780 nm.

16. The tunable lighting system of claim 15, wherein said violet channel is configured to emit unsaturated light having a spectral power distribution comprising a violet peak at a wavelength of between about 380 nm and about 460 nm, a violet valley at a wavelength of between about 450 nm and about 510 nm, a green peak at a wavelength of between about 5 nm and about 650 nm, and a red valley at a wavelength between about 650 nm and about 780 nm.

17. The tunable lighting system of claim 15, wherein said yellow light is configured to emit unsaturated light having a spectral power distribution comprising a Violet Peak (VP) at a wavelength of between about 330 nm and about 430 nm, a Violet Valley (VV) at a wavelength of between about 420 nm and about 510 nm, and a Green Peak (GP) at a wavelength of between about 50 nm and about 780 nm.

18. The tunable lighting system of claim 1, wherein said CCI range is at least 3500 K.

19. The tunable lighting system of claim 18, wherein said CCT range is at least 4000 K.

20. The tunable lighting system of claim 19, wherein said CCT range is between 2000 K and 6000 K.

21. The tunable lighting system of claim 20, wherein said CCT range is between 1800 K and 10,000 K.

77. The tunable lighting system of claim 1, wherein said emitted light has an R9 of at least 60.

23. The tunable lighting system of claim 22, wherein said emitted light has an R9 of at least 80.

24. The tunable lighting system of claim 1, wherein said CRI is at least 90.

25. The tunable lighting system of claim 24, wherein said CRI is at least 95.

26. The tunable lighting system of claim 1, wherein each channel comprises an InGaN LED.

27. The tunable lighting system of claim 1, wherein said second channel comprises a long blue LED pumping a cyan luminophoric.

28. The tunable lighting system of claim 1, wherein said third channel comprises a short blue LED pumping a cyan luminophoric.

29. The tunable lighting system of claim 1, wherein said multichannel driver drives a selection of at least three channels of said plurality of channels.

30. The tunable lighting system of claim 29, wherein said multichannel driver drives a selection of only three channels of said plurality of channels.

31. The tunable lighting system of claim 1, wherein a combination of said first, second, and fourth channels are driven to emit a high EML light.

32. The tunable lighting system of claim 15, wherein a combination of said fourth, fifth, and sixth channels are driven to emit a low EML light.

33. The tunable lighting system of claim 1, wherein a combination of said first, third and fourth channels are driven to emit a high CRI light.
